SEQUENCE LISTING (3 sequences)

SEQ ID NO: 1 - protein, 1367 aa, Homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 2 - DNA, 12235 bp, Homo sapiens
  source 1..12235: mol_type = unassigned DNA
agtgtgtggcagcggcggcggcggcgcggcgaggctggggctcttgtttaccagcattaactccgctgagcggaaaaaaaaagggaaaaaacccgaggaggagcgagcgcaccaggcgaactcgagagaggcgggagagcgagagggacgccgccagcgagcctgcccacggccggcgctcgcagaccctcggccccgctccccggatccccccgcgccctccacgcccctcccgcgcgggggcagctccacggcgcgcctcgcctcggctgtgaccttcagcgagccggagcccccgcgcagagcaggcggcggcgggcgggggccgggcgggggccggcgcggggcgggcggcggcgcagagccgggcggcgcggcgggagtgctgagcgcggcgcggccggcccgccgctttgtgtgtgtcctggatttgggaaggagctcgccgcggcggcggcggcgctgagggaggaggcggcggcgagcggagccaggaggaggaggaggagggggagccgctcattcattttgactccgcgtttctgcccctcgccggcctcgcctgtgacccggacttcggggcgatcttgcgaactgcgtcgcgccctcccgcggcggaagctcgggcgtccggccgcctcccgcgcggccagggccgggcttgtttttcctcgcctaggcagatttgggctttgccccctttctttgcagttttccccccttcctgcctctccgggtttgaaaatggaggccgacgacgccgacagcccgccccggcgcgcctcgggttcccgactccgccgagccctgggccgctgctgccggcgctgaggggccgccccgcgccgcccgccccgtccgcgcacccggagggccccggcggcgccgccttcggagtattgtttccttcgcccttgtttttggagggggagcgaagactgagtttgagacttgtttcctttcatttcctttttttcttttcttttcttttttttttttttttttttttttgagaaaggggaatttcatcccaaataaaaggaatgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctgatccttggatcctgaatctgtgcaaacagtaacgtgtgcgcacgcgcagcggggtggggggggagagagagttttaacaatccattcacaagcctcctgtacctcagtggatcttcagaactgcccttgctgcccgcgggagacagcttctctgcagtaaaacacatttgggatgttccttttttcaatatgcaagcagctttttattccctgcccaaacccttaactgacatgggcctttaagaaccttaatgacaacacttaatagcaacagagcacttgagaaccagtctcctcactctgtccctgtccttccctgttctccctttctctctcctctctgcttcataacggaaaaataattgccacaagtccagctgggaagccctttttatcagtttgaggaagtggctgtccctgtggccccatccaaccactgtacacacccgcctgacaccgtgggtcattacaaaaaaacacgtggagatggaaatttttacctttatctttcacctttctagggacatgaaatttacaaagggccatcgttcatccaaggctgttaccattttaacgctgcctaattttgccaaaatcctgaactttctccctcatcggcccggcgctgattcctcgtgtccggaggcatgggtgagcatggcagctggttgctccatttgagagacacgctggcgacacactccgtccatccgactgcccctgctgtgctgctcaaggccacaggcacacaggtctcattgcttctgactagattattatttgggggaactggacacaataggtctttctctcagtgaaggtggggagaagctgaaccggcttccctgccctgcctccccagccccctgcccaacccccaagaatctggtggccatgggccccgaagcagcctggcggacaggcttggagtcaaggggccccatgcctgcttctctcccagccccagctcccccgcccgcccccaaggacacagatgggaaggggtttccagggactcagccccactgttgatgcaggtttgcaaggaaagaaattcaaacaccacaacagcagtaagaagaaaagcagtcaatggattcaagcattctaagctttgttgacattttctctgttcctaggacttcttcatgggtcttacagttctatgttagaccatgaaacatttgcatacacatcgtctttaatgtcacttttataacttttttacggttcagatattcatctatacgtctgtacagaaaaaaaaaagctgctattttttttgttcttgatctttgtggatttaatctatgaaaaccttcaggtccaccctctcccctttctgctcactccaagaaacttcttatgctttgtactagagtgcgtgactttcttcctcttttcccggtaatggatacttctatcacataatttgccatgaactgttggatgcctttttataaatacatcccccatccctgctcccacctgcccctttagttgttttctaacccgtaggctctctgggcacgaggcagaaagcaggccgggcacccatcctgagagggccgcgctcctctccccagcctgccctcacagcattggagcctgttacagtgcaagacatgatacaaactcaggtcagaaaaacaaaggttaaatatttcacacgtctttgttcagtgtttccactcaccgtggttgagaagcctcaccctctctttcccttgcctttgcttaggttgtgacacacatatatatatatttttttaattcttgggtacaacagcagtgttaaccgcagacactaggcatttggattactatttttcttaatggctatttaatccttccatcccacgaaaaacagctgctgagtccaagggagcagcagagcgtggtccggcagggcctgttgtggccctcgccacccccctcaccggaccgactgacctgtctttggaaccagaacatcccaagggaactccttcgcactggcgttgagtgggaccccgggatccaggctggcccagggcggcaccctcagggctgtgcccgctggagtgctaggtggaggcagcacagacgccacggtggcccaagagcccctttgcttcttgctgggggaccagggctgtggtgctggcccactttccctcggccaggaatccaggtccttggggcccaggggtcttgtcttgtttcatttttagcacttctcaccagagagatgacagcacaagagttgcttctgggatagaaatgtttaggagtaagaacaaagctgggatacggtgattgctagttgtgactgaagattcaacacagaaaagaaagtttatacggcttttttgctggtcagcagtttgtcccactgctttctctagtctctatcccatagcgtgttccctttaaaaaaaaaaaaaaggtattatatgtaggagttttcttttaatttattttgtgataaattaccagtttcaatcactgtagaaaagccccattatgaatttaaatttcaaggaaagggtgtgtgtgtgtgtatgtgtggggtgtgtgtgtgtgagagtgatgggacagttcttgattttttgggttttttttcccccaaacatttatctacctcactcttattttttatatgtgtatatagacaaaagaatacatctcacctttctcagcacctgacaataggccgttgatactggtaacctcatccacgccacaggcgccacacccaggtgatgcagggggaagccaggctgtattccggggtcaaagcaacactaactcacctctctgctcatttcagacagcttgcctttttctgagatgtcctgttttgtgttgctttttttgttttgttttctatcttggtttccaccaaggtgttagatttctcctcctcctagccaggtggccctgtgaggccaacgagggcaccagagcacacctgggggagccaccaggctgtccctggctggttgtctttggaacaaactgcttctgtgcagatggaatgaccaacacatttcgtccttaagagagcagtggttcctcaggttctgaggagaggaaggtgtccaggcagcaccatctctgtgcgaatccccagggtaaaggcgtggggcattgggtttgctccccttgctgctgctccatccctgcaggaggctcgcgctgaggcaggaccgtgcggccatggctgctgcattcattgagcacaaaggtgcagctgcagcagcagctggagagcaagagtcacccagcctgtgcgccagaatgcagaggctcctgacctcacagccagtccctgatagaacacacgcaggagcagagtcccctccccctccaggctgccctctcaacttctccctcacctccttccctaggggtagacagagatgtaccaaaccttccggctggaaagcccagtggccggcgccgaggctcgtggcgtcacgccccccccgccagggctgtacctccgtctccctggtcctgctgctcacaggacagacggctcgctcccctcttccagcagctgctcttacaggcactgatgatttcgctgggaagtgtggcgggcagctttgcctaagcgtggatggctcctcggcaattccagcctaagtgaaggcgctcaggagcctcctgctggaacgcgacccatctctcccaggaccccggggatcttaaggtcattgagaaatactgttggatcagggttttgttcttccacactgtaggtgaccccttggaataacggcctctcctctcgtgcacatacctaccggtttccacaactggatttctacagatcattcagctggttataagggttttgtttaaactgtccgagttactgatgtcattttgtttttgttttatgtaggtagcttttaagtagaaaacactaacagtgtagtgcccatcatagcaaatgcttcagaaacacctcaataaaagagaaaacttggcttgtgtgatggtgcagtcactttactggaccaacccacccaccttgactataccaaggcatcatctatccacagttctagcctaacttcatgctgatttctctgcctcttgatttttctctgtgtgttccaaataatcttaagctgagttgtggcattttccatgcaacctccttctgccagcagctcacactgcttgaagtcatatgaaccactgaggcacatcatggaattgatgtgagcattaagacgttctcccacacagcccttccctgaggcagcaggagctggtgtgtactggagacactgttgaacttgatcaagacccagaccaccccaggtctccttcgtgggatgtcatgacgtttgacatacctttggaacgagcctcctccttggaagatggaagaccgtgttcgtggccgacctggcctctcctggcctgtttcttaagatgcggagtcacatttcaatggtacgaaaagtggcttcgtaaaatagaagagcagtcactgtggaactaccaaatggcgagatgctcggtgcacattggggtgctttgggataaaagatttatgagccaactattctctggcaccagattctaggccagtttgttccactgaagcttttcccacagcagtccacctctgcaggctggcagccgaatggcttgccagtggctctgtggcaagatcacactgagatcgatgggtgagaaggctaggatgcttgtctagtgttcttagctgtcacgttggctccttccagggtggccagacggtgttggccactcccttctaaaacacaggcgccctcctggtgacagtgacccgccgtggtatgccttggcccattccagcagtcccagttatgcatttcaagtttggggtttgttcttttcgttaatgttcctctgtgttgtcagctgtcttcatttcctgggctaagcagcattgggagatgtggaccagagatccactccttaagaaccagtggcgaaagacactttctttcttcactctgaagtagctggtggtacaaatgagaacttcaagagaggatgttatttagactgaacctctgttgccagagatgctgaagatacagaccttggacaggtcagagggtttcatttttggccttcatcttagatgactggttgcgtcatttggagaagtgagtgctccttgatggtggaatgaccgggtggtgggtacagaaccattgtcacagggatcctggcacagagaagagttacgagcagcagggtgcagggcttggaaggaatgtgggcaaggttttgaacttgattgttcttgaagctatcagaccacatcgaggctcagcagtcatccgtgggcatttggtttcaacaaagaaacctaacatcctactctggaaactgatctcggagttaaggcgaattgttcaagaacacaaactacatcgcactcgtcagttgtcagttctggggcatgactttagcgttttgtttctgcgagaacataacgatcactcatttttatgtcccacgtgtgtgtgtccgcatctttctggtcaacattgttttaactagtcactcattagcgttttcaatagggctcttaagtccagtagattacgggtagtcagttgacgaagatctggtttacaagaactaattaaatgtttcattgcatttttgtaagaacagaataattttataaaatgtttgtagtttataattgccgaaaataatttaaagacactttttttttctctgtgtgtgcaaatgtgtgtttgtgatccattttttttttttttttttaggacacctgtttactagctagctttacaatatgccaaaaaaggatttctccctgaccccatccgtggttcaccctcttttccccccatgctttttgccctagtttataacaaaggaatgatgatgatttaaaaagtagttctgtatcttcagtatcttggtcttccagaaccctctggttgggaaggggatcattttttactggtcatttccctttggagtgtagctactttaacagatggaaagaacctcattggccatggaaacagccgaggtgttggagcccagcagtgcatggcaccgttcggcatctggcttgattggtctggctgccgtcattgtcagcacagtgccatggacatgggaagacttgactgcacagccaatggttttcatgatgattacagcatacacagtgatcacataaacgatgacagctatggggcacacaggccatttgcttacatgcctcgtatcatgactgattactgctttgttagaacacagaagagaccctattttatttaaggcagaaccccgaagatacgtatttccaatacagaaaagaatttttaataaaaactataacatacacaaaaattggttttaaagttgactccacttcctctaactccagtggattgttggccatgtctccccaactccacaatatctctatcatgggaaacacctggggtttttgcgctacataggagaaagatctggaaactatttgggttttgttttcaacttttcatttggatgtttggcgttgcacacacacatccaccggtggaagagacgcccggtgaaaacacctgtctgctttctaagccagtgaggttgaggtgagaggtttgccagagtttgtctacctctgggtatccctttgtctgggataaaaaaaatcaaaccagaaggcgggatggaatggatgcaccgcaaataatgcattttctgagttttcttgttaaaaaaaaatttttttaagtaagaaaaaaaaaggtaataacatggccaatttgttacataaaatgactttctgtgtataaattattcctaaaaaatcctgtttatataaaaaatcagtagatgaaaaaaatttcaaaatgtttttgtatattctgttgtaagaatttattcctgttattgcgatatactctggattctttacataatggaaaaaagaaactgtctattttgaatggctgaagctaaggcaacgttagtttctcttactctgcttttttctagtaaagtactacatggtttaagttaaataaaataattctgtatgca

SEQ ID NO: 3 - DNA, 58 bp, Homo sapiens
  source 1..58: mol_type = unassigned DNA
cagcggcagcctcaggacggctacctttaccggcacaattactgctccaaaggtaagg

(19) United States
(12) Patent Application Publication
(10) Pub. No.: US 2026/0159894 A1
(43) Pub. Date: Jun. 11, 2026

(54) IGF1R ACTIVATION MUTATIONS AND USES THEREOF

(71) Applicant: Foundation Medicine, Inc., Boston, MA (US)

(72) Inventors: Brennan DECKER, Winchester, MA (US); Matthew MARGOLIS, Boston, MA (US); Tyler Williamson JANOVITZ, Brookline, MA (US)

(73) Assignee: Foundation Medicine, Inc., Boston, MA (US)

(21) Appl. No.: 18/958,995

(22) Filed: Nov. 25, 2024

Related U.S. Application Data
(63) Continuation of application No. PCT/US2023/067796, filed on Jun. 1, 2023.
(60) Provisional application No. 63/348,966, filed on Jun. 3, 2022.

(51) Int. Cl. C12Q 1/6886; G01N 33/575

(52) U.S. Cl. CPC C12Q 1/6886; G01N 33/5758; C12Q 2600/156; G01N 2333/71

(57) ABSTRACT

IGF1R polypeptides having an activating mutation, and nucleic acid molecules encoding said IGF1R polypeptides, are described herein. The activating mutation is located within an identified hotspot region of the IGF1R polypeptide. Activating mutations of the IGF1R polypeptide include a non-frameshift insertion mutation at or following any one or more of amino acids 653-667 of IGF1R or within a tyrosine kinase domain of IGF1R (e.g., at or following any one or more of amino acids 1034-1049 of IGF1R), or a substitution or insertion mutation at D555 of IGF1R. Also described are methods related to detecting such IGF1R mutants, as well as methods of diagnosis/treatment and uses related thereto.

### CROSS-REFERENCE TO RELATED APPLICATIONS

This application is a continuation application of International Application No. PCT/US2023/067796, filed internationally on Jun. 1, 2023, which claims the priority benefit of U.S. Provisional Patent Application Ser. No. 63/348,966, filed Jun. 3, 2022, the disclosure of each of which is hereby incorporated by reference in its entirety.

### REFERENCE TO AN ELECTRONIC SEQUENCE LISTING

The content of the electronic sequence listing (197102009501seglist.xml; Size: 17,291 bytes; and Date of Creation: Oct. 29, 2024) is herein incorporated by reference in its entirety.

### TECHNICAL FIELD

Described herein are methods of diagnosing and/or treating a cancer based on on a detected IGF1R mutation. Also provided herein are IGF1R polypeptides having an activating mutation, and polynucleotides encoding said IGF1R polypeptides, and methods related to detecting such IGF1R mutants, as well as methods of diagnosis/treatment and uses related thereto.

### BACKGROUND

Insulin-like growth factor 1 receptor (IGF1R) is a receptor tyrosine kinase that signals through the PI3K-AKT and Ras-MAPK pathways to promote cell survival and proliferation.

Adenoid cystic carcinoma (ACC) is among the most prevalent malignant salivary gland tumors and typically follows a slow but progressive clinical course with poor long-term prognosis. There is significant histological overlap between ACC and other basaloid salivary gland neoplasms, yielding challenging diagnostic dilemmas, particularly on small biopsies and fine needle aspirations. Recurrent genomic alterations of primary and metastatic ACC have been previously studied, although the majority of ACC cases harbor driver MYB or MYBL1 fusions, with NFIB as the most frequent partner gene. Activating alterations in the NOTCH gene family, genes that activate the Notch-signaling pathway (inclusive of SPEN and FBXW7), are common and associated with a worse prognosis.

### SUMMARY OF THE INVENTION

The present disclosure relates generally to detecting insulin-like growth factor 1 receptor (IGF1R) mutations in cancer, as well as methods of treatment, and uses related thereto. IGF1R activation is targetable with existing tyrosine kinase inhibitors (TKIs), but mutations in IGF1R had not previously been systematically characterized. The IGF1R mutations described herein localize to defined hotspot regions within IGF1R and can be used to identify cancers that may be particularly susceptible to an IGF1R-targeted therapy. Further, mutations within these defined hotspot regions are particularly prominent among adenoid cystic carcinoma (ACC), and identification of such mutations may be used characterize a cancer as an ACC, which is frequently mischaracterized by clinicopathology. Hotspot activator mutations of IGF1R include (1) a non-frameshift insertion mutation at or following any one or more of amino acids 653-667 of IGF1R, (2) a non-frameshift insertion mutation within the tyrosine kinase domain of IGF1R, or (3) a substitution or insertion mutation at D555 of IGF1R.

Described herein is a method for identifying an individual having a cancer for treatment with an insulin-like growth factor 1 receptor (IGF1R)-targeted therapy comprising detecting an insulin-like growth factor 1 receptor (IGF1R) polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Also described is a method of selecting a treatment for an individual having a cancer, comprising: detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and selecting the treatment, wherein the presence of said non-frameshift insertion mutation or said substitution mutation identifies the individual as one who may benefit from treatment with an IGF1R-targeted therapy.

Further described is a method of identifying one or more treatment options for an individual having a cancer comprising: detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and generating a report comprising one or more treatment options identified for the individual based at least in part on the detection of the IGF1R polypeptide having the mutation, or the nucleic acid molecule encoding the IGF1R polypeptide having the mutation, in the sample, wherein the one or more treatment options comprises administration of an IGF1R-targeted therapy.

Additionally, there is described a method of identifying a candidate treatment for a cancer in an individual in need thereof, comprising: performing DNA sequencing on a sample obtained from the individual to determine a sequencing mutation profile, wherein the sequencing mutation profile identifies the presence or absence of a nucleic acid molecule that encodes an IGF1R polypeptide having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R, and selecting a treatment for the individual based, at least in part, on the sequencing mutation profile, wherein the treatment comprises an IGF1R-targeted therapy.

Also described is a method of predicting survival of an individual having a cancer treated with a treatment comprising an IGF1R-targeted therapy, the method comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R, wherein responsive to the acquisition of said knowledge, the individual is predicted to have longer survival when treated with the treatment comprising the IGF1R-targeted therapy, as compared to an individual whose cancer does not have the IGF1R polypeptide having the mutation or the nucleic acid molecule encoding the IGF1R polypeptide having the mutation.

Further described is a method of monitoring, evaluating, or screening an individual having a cancer, comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; wherein responsive to the acquisition of said knowledge, the individual is predicted to have acquired resistance to a prior anti-cancer therapy administered to the individual, the individual is predicted to respond to an IGF1R-targeted therapy, and/or the individual is predicted to have poor prognosis, as compared to an individual whose cancer does not comprise the IGF1R polypeptide having the mutation or the nucleic acid molecule encoding the IGF1R polypeptide having the mutation.

In some embodiments of any of the above methods, the method further comprises administering an effective amount of the IGF1R-targeted therapy to the individual, thereby treating the cancer.

Also described is a method of detecting a nucleic acid molecule encoding an IGF1R polypeptide having a mutation in a sample from an individual having a cancer, wherein the mutation is a non-frameshift insertion mutation at or following any one or more of amino acids 653-667 of IGF1R or within a tyrosine kinase domain of IGF1R, or a substitution or insertion mutation at D555 of IGF1R, comprising: (a) providing a plurality of nucleic acid molecules obtained from a sample from the individual, wherein the plurality of nucleic acid molecules comprises a fragment of the nucleic acid molecule encoding the IGF1R polypeptide comprising the mutation; (b) optionally, ligating one or more adapters onto one or more nucleic acid molecules from the plurality of nucleic acid molecules; (c) optionally, amplifying the one or more ligated nucleic acid molecules from the plurality of nucleic acid molecules; (d) optionally, capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the nucleic acid molecule encoding the IGF1R polypeptide having the mutation; (f) analyzing the plurality of sequence reads for the presence or absence of the nucleic acid molecule encoding the IGF1R polypeptide having the mutation; and (g) based on the analyzing step, detecting the presence or absence of the nucleic acid molecule encoding the IGF1R polypeptide having the mutation in the sample. The one or more adapters may comprise amplification primers, flow cell adapter sequences, substrate adapter sequences, sample index sequences, or unique molecular identifier (UMI) sequences. The amplified nucleic acid molecules may be captured by hybridization with one or more bait molecules.

Further described is a method of detecting a nucleic acid molecule encoding an IGF1R polypeptide having a mutation in a sample from an individual having a cancer, wherein the mutation is a non-frameshift insertion mutation at or following any one or more of amino acids 653-667 of IGF1R or within a tyrosine kinase domain of IGF1R, or a substitution or insertion mutation at D555 of IGF1R, the method comprising: (a) providing the sample from the individual, wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising a nucleotide sequence encoding at least a portion of an IGF1R polypeptide having the mutation, thereby making an enriched sample; (e) sequencing, by a sequencer, the enriched sample, thereby producing a plurality of sequence reads; (f) analyzing the plurality of sequence reads for the presence or absence of the mutation in the nucleic acid molecule encoding the IGF1R polypeptide; and (g) detecting, based on the analyzing step, the presence or absence of the nucleic acid molecule encoding the IGF1R polypeptide having the mutation in the sample from the individual. Selectively enriching may comprise: (a) combining one or more bait molecules with the library, thereby hybridizing the one or more bait molecules to one or more nucleic acid molecules comprising a nucleotide sequence encoding a portion of the IGF1R polypeptide, and producing nucleic acid hybrids; and (b) isolating the nucleic acid hybrids to produce the enriched sample.

The sequencer used in any of the above methods may comprise a next-generation sequencer.

In some embodiments of any of the above methods, amplifying comprises performing a polymerase chain reaction (PCR) amplification technique, a non-PCR amplification technique, or an isothermal amplification technique.

Additionally, there is described a method of treating a cancer, or delaying the progression of cancer, in an individual, comprising: detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and administering an effective amount of an IGF1R-targeted therapy to the individual.

The IGF1R-targeted therapy of the above methods may be administered to the individual in response to a determination of the presence of the non-frameshift insertion mutation or the substitution mutation.

Also described herein is a method of identifying a cancer in an individual as an adenoid cystic carcinoma comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R, wherein the presence of the mutation in IGF1R indicates an increased likelihood that the cancer is an adenoid cystic carcinoma.

The cancer may have been previously identified as a cancer other than an adenoid cystic carcinoma. The method may further comprise identifying a basaloid appearance, presence of cribriform architecture, or stroma characteristic of an adenoid cystic carcinoma, within a histological image of the cancer. The method may further comprise administering an IGF1R-targeted therapy to the individual, thereby treating the cancer. The IGF1R-targeted therapy may be administered to the individual, for example, in response to a determination of the presence of the non-frameshift insertion mutation or the substitution mutation.

In some embodiments of the above methods, detecting comprises selectively enriching for one or more nucleic acid molecules in the sample comprising nucleotide sequences encoding a portion of the IGF1R polypeptide to produce an enriched sample. Selectively enriching may comprise: (a) combining one or more bait molecules with the sample, thereby hybridizing the one or more bait molecules to one or more nucleic acid molecules in the sample comprising a nucleotide sequence encoding a portion of the IGF1Rpolypeptide, thereby producing nucleic acid hybrids; and (b) isolating the nucleic acid hybrids to produce the enriched sample. The one or more bait molecules may comprise a capture nucleic acid molecule configured to hybridize to a nucleotide sequence corresponding to IGF1R. The capture nucleic acid molecule may comprise, for example, between about 10 and about 30 nucleotides, between about 50 and about 1000 nucleotides, between about 100 and about 500 nucleotides, between about 100 and about 300 nucleotides, or between about 100 and about 200 nucleotides.

In some embodiments of the above methods, the one or more bait molecules are conjugated to an affinity reagent or to a detection reagent. The affinity reagent may be, for example, an antibody, an antibody fragment, or biotin, or wherein the detection reagent is a fluorescent marker.

In some embodiments of the above methods, the capture nucleic acid molecule comprises a DNA, RNA, or mixed DNA/RNA molecule.

In some embodiments, of the above methods the selectively enriching comprises amplifying the one or more nucleic acid molecules comprising nucleotide sequences corresponding to IGF1R using a polymerase chain reaction (PCR) to produce an enriched sample.

In some embodiments of the above methods, the method further comprises sequencing the enriched sample.

In some embodiments of the above methods, the sample comprises a mixture of cancer nucleic acid molecules and non-cancer nucleic acid molecules. The cancer nucleic acid molecules may be derived, for example, from a tumor portion of a heterogeneous tissue biopsy sample, and the non-cancer nucleic acid molecules are derived from a normal portion of the heterogeneous tissue biopsy sample.

In some embodiments of the above methods, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

In some embodiments of the above methods, the sequencing comprises use of a massively parallel sequencing (MPS) technique, whole genome sequencing (WGS), whole exome sequencing, targeted sequencing, direct sequencing, or a Sanger sequencing technique; optionally wherein the sequencing comprises a massively parallel sequencing technique, and the massively parallel sequencing technique comprises next-generation sequencing (NGS).

In some embodiments of the above methods, the method further comprises generating a report, wherein the report: (a) indicates the presence or absence of the IGF1R polypeptide having the mutation or the nucleic acid molecule encoding the IGF1R polypeptide having the mutation in a sample from the individual; and/or (b) indicates a treatment or one or more treatment options identified or selected for the individual based at least in part on the detection of the IGF1R polypeptide having the mutation, or the nucleic acid molecule encoding the IGF1R polypeptide having the mutation, in the sample, wherein the one or more treatment options comprises administration of an IGF1R-targeted therapy.

In some embodiments of the above methods, the method further comprises generating a molecular profile for the individual, based, at least in part, on detecting or acquiring knowledge of the IGF1R polypeptide having the mutation, or the nucleic acid molecule encoding the IGF1R polypeptide having the mutation, in the sample from the individual. The molecular profile for the individual may further comprise, for example, results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile for the individual comprises results from a nucleic acid sequencing-based test.

In some embodiments of the above methods, the method further comprises selecting a treatment, administering a treatment, or applying a treatment to the individual based on the generated molecular profile, wherein the treatment comprises an IGF1R-targeted therapy.

In some embodiments of the above methods, the method further comprises generating a report, wherein the report comprises the molecular profile for the individual. The report may further comprise, for example, information on a treatment or one or more treatment options identified or selected for the individual based, at least in part, on the molecular profile for the individual, wherein the treatment or one or more treatment options comprise an IGF1R-targeted therapy.

In some embodiments of the above methods, the method further comprises providing the report to the individual, a caregiver, a healthcare provider, a physician, an oncologist, an electronic medical record system, a hospital, a clinic, a third-party payer, an insurance company, or a government office.

In some embodiments of the above methods, the individual is a human.

In some embodiments of the above methods, the method further comprises obtaining the sample from the individual.

In some embodiments of the above methods, the sample is obtained or derived from the cancer.

In some embodiments of the above methods, the sample comprises a tissue biopsy sample, a liquid biopsy sample, or a normal control. For example, the sample may be from a tumor biopsy, tumor specimen, or circulating tumor cell.

In some embodiments of the above methods, the sample is a liquid biopsy sample comprising blood, serum, plasma, cerebrospinal fluid, sputum, stool, urine, or saliva.

In some embodiments of the above methods, the sample comprises cells and/or nucleic acids from the cancer.

In some embodiments of the above methods, the sample comprises mRNA, DNA, circulating tumor DNA (ctDNA), cell-free DNA, or cell-free RNA from the cancer.

In some embodiments of the above methods, the sample is a liquid biopsy sample comprising circulating tumor cells (CTCs).

In some embodiments of the above methods, the sample is a liquid biopsy sample comprising cell-free DNA (cfDNA), circulating tumor DNA (ctDNA), or any combination thereof.

In some embodiments of the above methods, the IGF1R polypeptide having the mutation is detected in the sample by one or more of: immunoblotting, enzyme linked immunosorbent assay (ELISA), immunohistochemistry, or mass spectrometry.

Also provided herein is a method of treating an individual having cancer comprising: selecting the individual for treatment based on the cancer having an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and administering an effective amount of an IGF1R-targeted therapy to the selected individual.

In some embodiments of the above methods, the cancer is a breast cancer, a head and neck cancer, an adenoid cystic carcinoma, an anal cancer, a bladder cancer, a uterine cancer, a lung cancer, a skin cancer, a neuroendocrine cancer, an ovarian cancer, or a prostate cancer. The cancer is, in some instances, an adenoid cystic carcinoma. The cancer may be a basaloid cancer. The cancer is, in some instances, a head or neck cancer. The cancer is, in some instances, a salivary gland cancer. For example, the cancer may be a basaloid salivary gland cancer.

In some embodiments of the above methods, the cancer is a metastatic cancer. In some embodiments of the above methods, the cancer is a primary cancer.

In some embodiments of the above methods, the mutation is a non-frameshift insertion mutation at or following any one or more of amino acids 653-667 of IGF1R. For example, the mutation may be a non-frameshift insertion mutation at or following any one or more of amino acids 663-666 of IGF1R or 663-668 of IGF1R. The non-frameshift insertion mutation may comprise a duplication. The non-frameshift insertion mutation may comprise a duplication of Y662 and C663. The non-frameshift insertion mutation may comprise a delins mutation. In some embodiments, the mutation is a non-frameshift insertion mutation at or following any one or more of amino acids 653-667 of IGF1R, and the non-frameshift insertion mutation is 3 to 13 amino acids in length. The mutation may comprise, for example, a Q653_K665dup mutation, a Q653_S664dup mutation, a R659_C663dup mutation, a Y656_C663dup mutation, a L657_S664dup mutation, a N661_D666dup mutation, a Y662_S664dup mutation, a C663_S664insYRHNYC mutation, a C663_S664insRHNYC mutation, a C663_S664insYGYLYRHNYC mutation, a C663_S664insYRHNYC mutation, a C663_S664insYLYRHNYC mutation, S664_K665insYCS mutation, a S664_K665insRHNYCS mutation, a S664_K665insLYRHNYCS mutation, a K665_D666insGYCSK mutation, K665_D666insQDGYLYRHNYCSK mutation, or a D666_K667insDNYCSK mutation.

In some embodiments of the above methods, the mutation is the non-frameshift insertion mutation within the tyrosine kinase domain of IGF1R. For example, the non-frameshift insertion mutation within the tyrosine kinase domain of IGF1R may be at or follow any one of amino acids 1034-1049 of IGF1R. In some embodiments, the non-frameshift insertion mutation within the tyrosine kinase domain of IGF1R comprises a delins mutation. In some embodiments, the non-frameshift insertion mutation within the tyrosine kinase domain of IGF1R is 1 to 9 amino acids in length. In some embodiments, the mutation comprises a T1034_V1035insN mutation, a A1039dup mutation, a A1039_S1040insA mutation, a M1041_R1042insL mutation, a R1042_E1046dup, a E1043_R1044insK mutation, a R1044_I1045insM mutation, a R1044_I1045insR mutation, a I1045_E1046insD mutation, a E1046_F1047insRERIE mutation, a E1046_F1047insAAMRERIE mutation, a F1047delinsLL mutation, a L1048dup mutation, a L1048_N1049insL mutation, or a N1049delinsLD mutation.

In some embodiments of the above methods, the mutation is the substitution or insertion mutation at D555 of IGF1R. In some embodiments, the mutation is the substitution mutation at D555 of IGF1R. In some embodiments, the mutation is a D555A mutation, a D555Y mutation, a D555E mutation, a D555N mutation, or a D555G mutation. In some embodiments, the mutation is D555_L556insVD.

In some embodiments of the above methods, the cancer is negative for a MYB, MYBL1, or NOTCH1 driver mutation. In some embodiments, the cancer is negative for a MYB-NFIB fusion.

In some embodiments of the above methods, the IGF1R having the mutation comprises the tyrosine kinase domain, or a fragment of the tyrosine kinase domain, having a kinase activity. In some embodiments, the kinase activity is constitutive.

In some embodiments of the above methods, the IGF1R having the mutation is oncogenic.

In some embodiments of the above methods, the IGF1R having the mutation promotes cancer cell survival, angiogenesis, cancer cell proliferation, and any combination thereof.

In some embodiments of the above methods, the IGF1R-targeted therapy comprises one or more of a small molecule inhibitor, an antibody, an antibody fragment, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an IGF1R mutation, an IGF1R-targeted therapy being tested in a clinical trial, a treatment for cancer comprising an IGF1R mutation being tested in a clinical trial, or any combination thereof.

In some embodiments of the above methods, the IGF1R-targeted therapy specifically targets IGF1R.

In some embodiments of the above methods, the IGF1R-targeted therapy specifically targets an IGF1R ligand.

In some embodiments of the above methods, the IGF1R-targeted therapy comprises an antibody or fragment thereof. For example, the IGF1R-targeted therapy may comprise a monoclonal antibody.

In some embodiments of the above methods, the IGF1R-targeted therapy comprises cixutumumab, figitumumab, dalotuzumab, ganitumab, robatumumab, BMS-754807, NVP-ADW742, NVP-AEW541, OSI-906, teprotumumab, ceritinib, xentuzumab, AXL1717, IGF-MTX, WO101, FPI-1434, SCH717454, AVE1642, BIIB022, or MEDI-573.

In some embodiments of the above methods, the IGF1R-targeted therapy comprises a kinase inhibitor. The IGF1R-targeted therapy may comprise, for example, a tyrosine kinase inhibitor. The IGF1R-targeted therapy may comprise kinase inhibitor that inhibits kinase activity of the IGF1R polypeptide.

In some embodiments of the above methods, the IGF1R-targeted therapy comprises a multi-kinase inhibitor.

In some embodiments of the above methods, the IGF1R-targeted therapy comprises an IGF1R-kinase specific inhibitor.

In some embodiments of the above methods, the IGF1R-targeted therapy comprises a small-molecule tyrosine kinase inhibitor. The small-molecule tyrosine kinase inhibitor may be, for example, linsitinib, ceritinib, BMS-754807, BVP 51004, XL228, or INSM-18.

In some embodiments of the above methods, the IGF1R-targeted therapy comprises a nucleic acid that inhibits the expression of IGF1R. The nucleic acid may be, for example, a double-stranded RNA (dsRNA), a small interfering RNA (siRNA), or a small hairpin RNA (shRNA).

In some embodiments of the above methods, the IGF1R-targeted therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments of the above methods, the individual has received a prior anti-cancer treatment, or is being treated with an anti-cancer treatment; optionally wherein the cancer is resistant or refractory to the anti-cancer treatment.

In some embodiments of the above methods, the treatment or the one or more treatment options further comprise an additional anti-cancer therapy. The additional anti-cancer therapy may comprise, for example, one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. The cellular therapy may be, for example, an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. The nucleic acid may comprise, for example, a double-stranded RNA (dsRNA), a small interfering RNA (siRNA), or a small hairpin RNA (shRNA).

Also described herein is a kit comprising one or more probes, baits, or oligonucleotides for detecting an IGF1R polypeptide, or a fragment thereof, having a mutation or nucleic acid molecule encoding the IGF1R polypeptide, or fragment thereof, having the mutation in a sample, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Further described herein is an isolated nucleic acid encoding an IGF1R polypeptide, or a fragment thereof, having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R. Further described is a vector comprising the nucleic acid. Also described is a host cell comprising the vector.

Further described herein is an antibody or antibody fragment that specifically binds to encoding an IGF1R polypeptide, or a fragment thereof, having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Also described is an IGF1R-targeted therapy for use in a method of treating or delaying progression of cancer, wherein the method comprises administering the IGF1R-targeted therapy to an individual having cancer, wherein an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R, is detected in a sample obtained from the individual.

Further described is an IGF1R-targeted therapy for use in the manufacture of a medicament for treating or delaying progression of cancer, wherein the medicament is to be administered to an individual having cancer, wherein an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R, is detected in a sample obtained from the individual.

Additionally, there is described a system, comprising: a memory configured to store one or more programs, and one or more processors configured to execute the one or more programs, the one or more programs when executed by the one or more processors are configured to: (a) obtain a plurality of sequence reads of one or more nucleic acid molecules, wherein the one or more nucleic acid molecules are derived from a sample obtained from an individual having a cancer; (b) analyze the plurality of sequence reads for the presence of a nucleic acid molecule encoding an IGF1R polypeptide having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and (c) detect, based on the analyzing, the nucleic acid molecule encoding the IGF1R polypeptide having the mutation in the sample.

Also described is a non-transitory computer readable storage medium comprising one or more programs executable by one or more computer processors for performing a method, the method comprising: (a) obtain a plurality of sequence reads of one or more nucleic acid molecules, wherein the one or more nucleic acid molecules are derived from a sample obtained from an individual having a cancer; (b) analyze the plurality of sequence reads for the presence of a nucleic acid molecule encoding an IGF1R polypeptide having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and (c) detect, based on the analyzing, the nucleic acid molecule encoding the IGF1R polypeptide having the mutation in the sample.

The plurality of sequence reads of the above system or non-transitory computer readable storage medium may be obtained by sequencing. The sequencing may comprise use of a massively parallel sequencing (MPS) technique, whole genome sequencing (WGS), whole exome sequencing, targeted sequencing, direct sequencing, or a Sanger sequencing technique. For example, the sequencing may comprise use of a massively parallel sequencing (MPS) technique, and wherein the massively parallel sequencing technique comprises next generation sequencing (NGS).

The one or more programs of the system or the non-transitory computer readable storage medium may be further configured to generate, based at least in part on the detecting, a molecular profile for the sample. The molecular profile may further comprise results from a comprehensive genomic profiling (CGP) test, a gene expression profiling test, a cancer hotspot panel test, a DNA methylation test, a DNA fragmentation test, an RNA fragmentation test, or any combination thereof. The molecular profile may further comprise results from a nucleic acid sequencing-based test.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings.

FIG. 1 provides a schematic overview of an exemplary study design and data analysis workflow for the identification of IGF1R driver mutations. Comprehensive genomic profiles (CGPs), including those with and without IGF1R alterations, were retrieved from a database for a retrospective analysis. Duplicate samples, subjects that did not consent to research, samples with significant contamination (including from transplants), tumor mutational burden (TMB)-high (TMB≥10 mut/Mb), and samples profiled using a method other than one of two targeted sequencing methods were excluded from further analysis. In addition, IGF1R variants with unknown or predicted benign functional status were filtered from the cohort.

FIG. 2 shows exemplary IGF1R mutations identified from a pan-cancer tumor sample analysis. The upper track displays single nucleotide variants and truncating insertions and deletions (delins), whereas the lower track shows non-frameshift insertion and deletion mutations.

FIG. 3 shows the frequency of IGF1R mutations by cancer type, stratified by mutation type. Short variants include single nucleotide substitutions and delins. ACC=Adenoid cystic carcinoma; CNS=Central nervous system; CUP=Carcinoma of unknown primary; NSCLC=Non-small cell lung carcinoma.

FIG. 4 shows a bar graph of the frequency of IGF1R non-frameshift mutations by cancer type, which compares the percent of tumor samples with a non-frameshift variation or a non-frameshift hotspot insertion across the top 12 tumor types that display the highest number of non-frameshift mutations. Only cancer types with ≥1,000 samples and at least two total IGF1R non-frameshift alterations are shown.

FIG. 5 depicts the co-occurrence patterns for ACCs, stratified by driver alteration status (MYB fusion, NOTCH1, IGF1R). ACC=Adenoid cystic carcinoma; TMB=Tumor mutational burden (mutations/Mb).

FIG. 6 shows the spectrum of IGF1R somatic single nucleotide and delins mutations in ACC. Non-frameshift delins were concentrated in hotspots at amino acids 663-666 and 1034-1049.

FIG. 7 depicts the configuration of non-frameshift duplication events in exon 9 (SEQ ID NO: 3) of IGF1R for ACC (upper) and non-ACC (lower) samples. Between three and 13 amino acids were duplicated and all included Y262 and C263 (red dashed box).

FIG. 8A shows representative hematoxylin & eosin (H&E) staining of a salivary neoplasm cross-section from a tumor that was found to carry at least one IGF1R hotspot single nucleotide variant. This tumor specimen carried an IGF1R hotspot non-frameshift insertion.

FIG. 8B shows representative hematoxylin & eosin (H&E) staining of a salivary neoplasm cross-section from a tumor that was found to carry at least one IGF1R hotspot single nucleotide variant. This tumor specimen carried an IGF1R hotspot in-frame delins alteration.

FIG. 8C shows representative hematoxylin & eosin (H&E) staining of a salivary neoplasm cross-section from a tumor that was found to carry at least one IGF1R hotspot single nucleotide variant. This tumor specimen carried an IGF1R hotspot non-frameshift insertion.

FIG. 8D shows representative hematoxylin & eosin (H&E) staining of a salivary neoplasm cross-section from a tumor that was found to carry at least one IGF1R hotspot single nucleotide variant. This tumor specimen carried an IGF1R hotspot in-frame delins alteration.

FIG. 9 depicts an exemplary device, in accordance with some embodiments.

FIG. 10 depicts an exemplary system, in accordance with some embodiments.

FIG. 11 depicts a block diagram of an exemplary process for detecting a nucleic acid molecule that encodes an IGF1R polypeptide having an activating mutation, in accordance with some embodiments.

### DETAILED DESCRIPTION

The present disclosure relates generally to detecting insulin-like growth factor 1 receptor (IGF1R) mutations in cancer, as well as methods of treatment, and uses related thereto. IGF1R activation is targetable with existing tyrosine kinase inhibitors (TKIs), but mutations in IGF1R had not previously been systematically characterized. The IGF1R mutations described herein localize to defined hotspot regions within IGF1R and can be used to identify cancers that may be particularly susceptible to an IGF1R-targeted therapy. Further, mutations within these defined hotspot regions are particularly prominent among adenoid cystic carcinoma (ACC), and identification of such mutations may be used characterize a cancer as an ACC, which is frequently mischaracterized by clinicopathology. Hotspot activator mutations of IGF1R include (1) a non-frameshift insertion mutation at or following any one or more of amino acids 653-667 of IGF1R, (2) a non-frameshift insertion mutation within the tyrosine kinase domain of IGF1R, or (3) a substitution or insertion mutation at D555 of IGF1R.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. DEFINITIONS

As used in this specification and the appended claims, the singular forms “a”, “an” and “the” include plural referents unless the context clearly dictates otherwise. Thus, for example, reference to “a molecule” optionally includes a combination of two or more such molecules, and the like.

The terms “about” and “approximately” as used herein refer to the usual error range for the respective value readily known to the skilled person in this technical field. Exemplary degrees of error are within 20 percent (%), typically, within 10%, and more typically, within 5% of a given value or range of values. Reference to “about” or “approximately” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and Y terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one that has been identified and separated and/or recovered from a component of its natural environment. Contaminant components of its natural environment are materials that would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“x”) and lambda (“V”), based on the amino acid sequences of their constant domains.

The term “constant domain” refers to the portion of an immunoglobulin molecule having a more conserved amino acid sequence relative to the other portion of the immunoglobulin, the variable domain, which contains the antigen-binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) in both the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain that are hypervariable in sequence and/or form structurally defined loops. Generally, antibodies comprise six HVRs: three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1 993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-1 13 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-31 0 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 1 1 9-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1 993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one that possesses an amino acid sequence that corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one that inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

The terms “homology” or “identity,” as used herein, refer to sequence similarity between two polynucleotide sequences or between two polypeptide sequences. The phrases “percent identity or homology” and “% identity or homology” refer to the percentage of sequence similarity found in a comparison of two or more polynucleotide sequences or two or more polypeptide sequences. Identity or similarity can be determined by comparing a position in each sequence that can be aligned for purposes of comparison. When a position in the compared sequences is occupied by the same nucleotide base or amino acid, then the molecules are identical at that position.

The term “detection” includes any means of detecting, including direct and indirect detection. The term “biomarker” as used herein (e.g., a “biomarker” such as an IGF1R polypeptide having an activating mutation, or a nucleic acid molecule encoding an IGF1R polypeptide having an activating mutation) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., an IGF1R polypeptide having an activating mutation, or a nucleic acid molecule encoding an IGF1R polypeptide having an activating mutation, described herein) described herein. In certain embodiments, the manufacture or kit is promoted, distributed, or sold as a unit for performing the methods described herein.

The phrase “based on”, “responsive to”, and the like, when used herein mean that the information about one or more biomarkers (e.g., an IGF1R mutant polypeptide, or nucleic acid molecule encoding said IGF1R mutant polypeptide, described herein) is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

An “IGF1R-targeted therapy” refers to any anti-cancer therapy that targets IGF1R, either specifically or non-specifically, or modulates IGF1R activity (binding activity, kinase activity, dimerization activity, or other biological activity) or expression, or that targets, either specifically or nonspecifically, or modulates biological activity or expression of any biological molecule upstream or downstream of IGF1R within the IGF1R signaling axis.

An “insertion” refers to a sequence change between translation initiation (start) codon and termination (stop) codon, where compared to a reference sequence, one or more amino acids are inserted, which is not a frame shift. An insertion “following” an indicated codon or amino acid refers to an insertion immediately following the indicated codon or amino acid (i.e., with no intervening codons or amino acids). Codon or amino acid numbering is made in reference to a polypeptide sequence of IGF1R according to SEQ ID NO: 1. Numbering may also or alternatively be made in reference to a nucleic aid sequence of IGF1R according to SEQ ID NO: 2. Although SEQ ID NO: 1 and SEQ ID NO: 2 provide polypeptide and nucleic acid reference sequences for IGF1R, it is understood that a specific IGF1R sequence from an individual may include one more additional variants that shift the amino acid, codon, or nucleotide numbering; nevertheless, one can readily correspond codon, amino acid, or nucleotide numbering for a particular IGF1R sequence to the IGF1R reference sequence of SEQ ID NO: 1 or SEQ ID NO:2. Unless otherwise specified, the term “insertion” includes a “duplication,” which refers to sequence change between translation initiation (start) codon and termination (stop) codon, where compared to a reference sequence, a copy of one or more amino acids are inserted directly C-terminal of the original copy of that sequence. Further, unless otherwise specified, the term “insertion” includes an “insertion-deletion” (also referred to as a “deletion-insertion”) or “delins,” which refers to a sequence change between translation initiation (start) codon and termination (stop) codon, where compared to a reference sequence, one or more amino acids are replaced with one or more other amino acids which is not a single-base substitution, a frame shift, or a conversion, which may, in some embodiments, cause the total number of amino acids at the mutation site to increase. A delins “at” an indicated codon(s) or amino acid(s) refers to a delins that replaces the indicated codon(s) or amino acid(s).

Nomenclature for insertions duplications, delins, and substitutions follows the guidelines provided by the convention provided by the Human Genome Variation Society, version 20.05.

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### III. METHODS, SYSTEMS, AND DEVICES

In certain aspects, provided herein are methods for selecting a treatment for an individual having a cancer; methods for identifying one or more treatment options for an individual having a cancer; methods for predicting survival of an individual having a cancer; methods for treating or delaying progression of cancer; methods for monitoring, evaluating or screening an individual having a cancer; methods for assessing an IGF1R nucleic acid molecule having an activating mutation or an IGF1R polypeptide having an activating mutation in a cancer in an individual; methods for detecting an IGF1R nucleic acid molecule having an activating mutation or an IGF1R polypeptide having an activating mutation; methods for detecting the presence or absence of a cancer in an individual; methods for monitoring progression or recurrence of a cancer in an individual; methods for identifying a candidate treatment for a cancer in an individual in need thereof; methods for identifying an individual having a cancer who may benefit from a treatment comprising an anti-cancer therapy, such as an IGF1R-targeted therapy; methods for predicting survival of an individual having a cancer treated with a treatment comprising an anti-cancer therapy, such as an IGF1R-targeted therapy; and methods of identifying a cancer in an individual as an adenoid cystic carcinoma based, at least in part, on the cancer having an IGF1R nucleic acid molecule having an activating mutation or an IGF1R polypeptide having an activating mutation.

In some embodiments, the methods provided herein comprise detecting in a sample from an individual, e.g., an individual having cancer, suspected of having cancer, being treated for cancer, or being tested for cancer, an IGF1R polypeptide having an activating mutation or a nucleic acid molecule encoding said IGF1R polypeptide having said activating mutation. In some embodiments, detection of the IGF1R polypeptide having an activating mutation or a nucleic acid molecule encoding said IGF1R polypeptide having said activating mutation, in the sample, identifies the individual as one who may benefit from a treatment comprising an anti-cancer therapy, such as an IGF1R-targeted therapy. In some embodiments, the methods comprise selecting an anti-cancer therapy as a treatment for an individual having cancer, e.g., responsive to detection of an IGF1R polypeptide having an activating mutation or a nucleic acid molecule encoding said IGF1R polypeptide having said activating mutation, in a sample from an individual, e.g., an individual having cancer, suspected of having cancer, being treated for cancer, or being tested for cancer. In some embodiments, the methods comprise generating a report comprising one or more treatment options identified for an individual (e.g., an individual having cancer, suspected of having cancer, being treated for cancer, or being tested for cancer) based at least in part on detection an IGF1R polypeptide having an activating mutation, or a nucleic acid molecule encoding said IGF1R polypeptide having said activating mutation, in a sample from the individual. In some embodiments, the one or more treatment options comprise an anti-cancer therapy as described herein, such as an IGF1R-targeted therapy. In some embodiments, the methods comprise administering to an individual (e.g., an individual having cancer, suspected of having cancer, being treated for cancer, or being tested for cancer) an effective amount of a treatment that comprises an anti-cancer therapy, such as an IGF1R-targeted therapy, responsive to detecting an IGF1R polypeptide having an activating mutation, or a nucleic acid molecule encoding said IGF1R polypeptide having said activating mutation, in a sample from the individual. In some embodiments, responsive to detection of an IGF1R polypeptide having an activating mutation, or a nucleic acid molecule encoding said IGF1R polypeptide having said activating mutation, in a sample from an individual (e.g., an individual having cancer, suspected of having cancer, being treated for cancer, or being tested for cancer), the individual is predicted to have longer survival when treated with a treatment comprising an anti-cancer therapy, such as an IGF1R-targeted therapy, as compared to survival of an individual whose cancer does not comprise an IGF1R polypeptide having an activating mutation, or a nucleic acid molecule encoding said IGF1R polypeptide having said activating mutation. In some embodiments, the methods comprise providing an assessment of an IGF1R polypeptide having an activating mutation, or a nucleic acid molecule encoding said IGF1R polypeptide having said activating mutation, e.g., responsive to detecting the presence or absence of an IGF1R polypeptide having an activating mutation, or a nucleic acid molecule encoding said IGF1R polypeptide having said activating mutation, in a sample. In some embodiments, the methods comprise detecting or acquiring knowledge of the presence or absence of a cancer in a sample from the individual. In some embodiments, the methods comprise detecting, in a first sample obtained from an individual (e.g., an individual having cancer, suspected of having cancer, being treated for cancer, or being tested for cancer) at a first time point, the presence or absence of an IGF1R polypeptide having an activating mutation, or a nucleic acid molecule encoding said IGF1R polypeptide having said activating mutation; detecting, in a second sample obtained from the individual at a second time point after the first time point, the presence or absence of an IGF1R polypeptide having an activating mutation, or a nucleic acid molecule encoding said IGF1R polypeptide having said activating mutation; and providing an assessment of cancer progression or cancer recurrence in the individual based, at least in part, on the presence or absence of the an IGF1R polypeptide having an activating mutation, or a nucleic acid molecule encoding said IGF1R polypeptide having said activating mutation, in the first sample and/or in the second sample. In some embodiments, the methods comprise performing DNA sequencing on a sample obtained from an individual to determine a sequencing mutation profile, wherein the sequencing mutation profile identifies the presence or absence of a nucleic acid molecule encoding an IGF1R polypeptide having an activating mutation. In some embodiments, the methods comprise identifying a candidate treatment based, at least in part, on the sequencing mutation profile. In some embodiments, the candidate treatment comprises an anti-cancer therapy described herein, such as an IGF1R-targeted therapy. In some embodiments, the candidate treatment is identified based, at least in part, on the presence of a nucleic acid molecule encoding an IGF1R polypeptide having an activating mutation as identified in the sequencing mutation profile.

In some embodiments, the methods provided herein comprise acquiring knowledge of an IGF1R polypeptide having an activating mutation, or a nucleic acid molecule encoding an IGF1R polypeptide having an activating mutation, in a sample from an individual, e.g., an individual having cancer, suspected of having cancer, being treated for cancer, or being tested for cancer. In some embodiments, knowledge of the presence of an IGF1R polypeptide having an activating mutation, or a nucleic acid molecule encoding an IGF1R polypeptide having an activating mutation, in a sample from an individual (e.g., an individual having cancer, suspected of having cancer, being treated for cancer, or being tested for cancer) identifies the individual as one who may benefit from a treatment comprising an anti-cancer therapy, such as an IGF1R-targeted therapy. In some embodiments, the methods comprise selecting an anti-cancer therapy, such as an IGF1R-targeted therapy, as a treatment for an individual (e.g., an individual having cancer, suspected of having cancer, being treated for cancer, or being tested for cancer), e.g., responsive to knowledge of the presence of an IGF1R polypeptide having an activating mutation, or a nucleic acid molecule encoding an IGF1R polypeptide having an activating mutation, in a sample from the individual. In some embodiments, the methods comprise generating a report comprising one or more treatment options identified for an individual (e.g., an individual having cancer, suspected of having cancer, being treated for cancer, or being tested for cancer) based at least in part on knowledge of the presence of an IGF1R polypeptide having an activating mutation, or a nucleic acid molecule encoding an IGF1R polypeptide having an activating mutation, in sample from the individual. In some embodiments, the one or more treatment options comprise an anti-cancer therapy described herein, such as an IGF1R-targeted therapy. In some embodiments, responsive to acquisition of knowledge of the presence of an IGF1R polypeptide having an activating mutation, or a nucleic acid molecule encoding an IGF1R polypeptide having an activating mutation, in a sample from an individual (e.g., an individual having cancer, suspected of having cancer, being treated for cancer, or being tested for cancer), the individual is classified as a candidate to receive a treatment comprising an anti-cancer therapy, e.g., such as an IGF1R-targeted therapy. In some embodiments, responsive to acquisition of knowledge of the presence of an IGF1R polypeptide having an activating mutation, or a nucleic acid molecule encoding an IGF1R polypeptide having an activating mutation, in a sample from an individual (e.g., an individual having cancer, suspected of having cancer, being treated for cancer, or being tested for cancer), the individual is identified as likely to respond to a treatment that comprises an anti-cancer therapy, such as an IGF1R-targeted therapy. In some embodiments, responsive to acquisition of knowledge of the presence an IGF1R polypeptide having an activating mutation, or a nucleic acid molecule encoding an IGF1R polypeptide having an activating mutation, in a sample from an individual, the individual is predicted to have longer survival when treated with a treatment comprising an anti-cancer therapy, such as an IGF1R-targeted therapy, e.g., as compared to survival of an individual whose cancer does not comprise an IGF1R polypeptide having an activating mutation, or a nucleic acid molecule encoding an IGF1R polypeptide having an activating mutation. In some embodiments, responsive to acquisition of knowledge of the presence of an IGF1R polypeptide having an activating mutation, or a nucleic acid molecule encoding an IGF1R polypeptide having an activating mutation, in a sample from an individual (e.g., an individual having cancer, suspected of having cancer, being treated for cancer, or being tested for cancer), the individual is predicted to have longer survival when treated with a treatment comprising an anti-cancer therapy, such as an IGF1R-targeted therapy, as compared to an individual whose cancer does not exhibit an IGF1R polypeptide having an activating mutation, or a nucleic acid molecule encoding an IGF1R polypeptide having an activating mutation. In some embodiments, the methods comprise administering to an individual (e.g., an individual having cancer, suspected of having cancer, being treated for cancer, or being tested for cancer) an effective amount of a treatment that comprises an anti-cancer therapy, such as an IGF1R-targeted therapy, responsive to acquiring knowledge an IGF1R polypeptide having an activating mutation, or a nucleic acid molecule encoding an IGF1R polypeptide having an activating mutation, in a sample from the individual. In some embodiments, responsive to acquiring knowledge of an IGF1R polypeptide having an activating mutation, or a nucleic acid molecule encoding an IGF1R polypeptide having an activating mutation, in a sample from an individual, the individual is predicted to have an improved response to treatment with an anti-cancer therapy, such as an IGF1R-targeted therapy, as compared to an individual whose cancer does not comprise an IGF1R polypeptide having an activating mutation, or a nucleic acid molecule encoding an IGF1R polypeptide having an activating mutation. In some embodiments, the methods comprise providing an assessment an IGF1R polypeptide having an activating mutation, or a nucleic acid molecule encoding an IGF1R polypeptide having an activating mutation, e.g., responsive to acquiring knowledge of the presence or absence of the IGF1R polypeptide, or a nucleic acid molecule encoding said IGF1R polypeptide, in a sample from an individual (e.g., an individual having cancer, suspected of having cancer, being treated for cancer, or being tested for cancer). In some embodiments, the methods comprise detecting or acquiring knowledge of the presence or absence of a cancer in a sample from an individual.

In other aspects, provided herein are systems. In some embodiments, a system of the disclosure comprises a memory configured to store one or more program instructions; and one or more processors configured to execute the one or more program instructions. In some embodiments, the one or more program instructions when executed by the one or more processors are configured to: (a) obtain a plurality of sequence reads of one or more nucleic acid molecules, wherein the one or more nucleic acid molecules are derived from a sample obtained from an individual (e.g., an individual having cancer, suspected of having cancer, being treated for cancer, or being tested for cancer); (b) analyze the plurality of sequence reads for the presence of a nucleic acid molecule encoding an IGF1R polypeptide having an activating mutation; and (c) detect, based on the analyzing, the nucleic acid molecule encoding an IGF1R polypeptide having an activating mutation in the sample.

In other aspects, provided herein are non-transitory computer readable storage media. In some embodiments, a non-transitory computer readable storage medium of the disclosure comprises one or more programs executable by one or more computer processors for performing a method. In some embodiments, the method comprises (a) obtaining, using the one or more processors, a plurality of sequence reads of one or more nucleic acid molecules, wherein the one or more nucleic acid molecules are derived from a sample obtained from an individual (e.g., an individual having cancer, suspected of having cancer, being treated for cancer, or being tested for cancer); (b) analyzing, using the one or more processors, the plurality of sequence reads for the presence of a nucleic acid molecule encoding an IGF1R polypeptide having an activating mutation; and (c) detecting, using the one or more processors and based on the analyzing, the nucleic acid molecule encoding the IGF1R polypeptide having the activating mutation, in the sample.

### A. IGF1R Activating Mutations

Three hotspot regions of IGF1R have been identified as being associated with cancer, and particularly adenoid cystic carcinoma (ACC). Among salivary gland tumors, IGF1R hotspot mutations were entirely specific to ACCs. Thus, the IGF1R mutation may be oncogenic. For example, the IGF1R mutation may promote cancer cell survival, angiogenesis, cancer cell proliferation, or any combination thereof. Further, in many cases, the identified IGF1R hotspot mutations were mutually exclusive with other known ACC drivers. Mutations within these hotspot regions have also been identified in other cancer types with lesser frequency, indicating that mutations within the hotspot regions of IGF1R are likely oncogenic driver mutations, and that this activation is under particular positive selection in the setting of ACC. Mutations may also be used to classify other cancer types, for example a breast cancer, a head and neck cancer, an anal cancer, a bladder cancer, a uterine cancer, a lung cancer, a skin cancer, a neuroendocrine cancer, an ovarian cancer, or a prostate cancer. IGF1R activating mutations can be targeted using an IGF1R-targeted therapy, as discussed herein. The activating mutation of IGF1R may be one or more of (1) a non-frameshift insertion mutation at or following any one or more of amino acids 653-667 of IGF1R, (2) a non-frameshift insertion mutation within a tyrosine kinase domain of IGF1R, or (3) a substitution or insertion mutation at D555 of IGF1R. The IGF1R having the mutation can include a tyrosine kinase domain, or a fragment of the tyrosine kinase domain, which has a kinase activity. The kinase activity may be constitutive, which may be attributable to the mutation.

The mutation in IGF1R may be a non-frameshift insertion mutation at or following any one or more of amino acids 653-667 (i.e., at or following amino acid 653, 654, 655, 656, 657, 658, 659, 660, 661, 662, 663, 664, 665, 666, or 667). Even more commonly, the non-frameshift insertion mutation is at or following any one or more of amino acids 663-666 (at or following amino acid 663, 664, 665, or 666). This hotspot region corresponds to a linker region connecting two α-CT motifs found at the end of a fibronectin type 3 domain in exon 9 of IGFR3. With respect to the nucleic acid sequence encoding the IGF1R polypeptide, the hotspot region is located with exon 9. It is believed that lengthening this region of IGF1R may increase binding of IGF1 to IGF1R and reduce the negative cooperativity, which operates to drive oncogenesis. The non-frameshift insertion mutation may be 1, 2, 3, 4, 5, 6, 7, 8, 9, 10, 11, 12, 13, 14, 15, or more amino acids in length. For example, the non-frameshift insertion mutation may be 3 to 13 amino acids in length. In some embodiments, the non-frameshift insertion mutation increases the length of the IGF1R polypeptide by 1, 2, 3, 4, 5, 6, 7, 8, 9, 10, 11, 12, 13, 14, 15, or more amino acids. For example, the non-frameshift insertion mutation may increase the length of the IGF1R polypeptide by 3 to 13 amino acids. In some instances, the non-frameshift insertion mutation is a duplication. In some instances, the non-frameshift insertion mutation is a near duplication (i.e., a delins that would be a duplication but for a single amino acid substitution). The non-frameshift insertion mutation may include a duplication of Y662 and C663 of IGF1R. Exemplary mutations within this hotspot region include, but are not limited to, a Q653_K665dup mutation, a Q653_S664dup mutation, a R659_C663dup mutation, a Y656_C663dup mutation, a L657_S664dup mutation, a N661_D666dup mutation, a Y662_S664dup mutation, a C663_S664insYRHNYC mutation, a C663_S664insRHNYC mutation, a C663_S664insYGYLYRHNYC mutation, a C663_S664insYRHNYC mutation, a C663_S664insYLYRHNYC mutation, S664_K665insYCS mutation, a S664_K665insRHNYCS mutation, a S664_K665insLYRHNYCS mutation, a K665_D666insGYCSK mutation, K665_D666insQDGYLYRHNYCSK mutation, and a D666_K667insDNYCSK mutation

The mutation of the IGF1R may be within the tyrosine kinase domain of IGF1R. The tyrosine kinase domain of IGF1R spans amino acids 999-1274 in exons 16-21 of IGF1R. More commonly, the non-frameshift insertion mutation is at or following an amino acid within the N-terminal end of the kinase domain (e.g., at or following any one or more amino acids 999-1049 of IGF1R). Even more commonly, the non-frameshift insertion mutation is at or following any one or more of amino acids 1034-1049 of IGF1R (for example, at or following one or more of amino acid 1034, 1035, 1036, 1037, 1038, 1039, 1040, 1041, 1042, 1043, 1044, 1045, 1046, 1047, 1048, or 1049). The non-frameshift insertion mutation may be 1, 2, 3, 4, 5, 6, 7, 8, 9, or more amino acids in length. For example, the non-frameshift insertion mutation may be 1 to 9 amino acids in length, or 1 to 5 amino acids in length. In some embodiments, the non-frameshift insertion mutation increases the length of the IGF1R polypeptide by 1, 2, 3, 4, 5, 6, 7, 8, 9, or more amino acids. For example, the non-frameshift insertion mutation may increase the length of the IGF1R polypeptide by 1 to 9 amino acids, or 1 to 5 amino acids. In some instances, the non-frameshift insertion mutation is a duplication. In some instances, the non-frameshift insertion mutation is a near duplication (i.e., a delins that would be a duplication but for a single amino acid substitution). Exemplary mutations within this hotspot region include, but are not limited to, a T1034_V1035insN mutation, a A1039dup mutation, a A1039_S1040insA mutation, a M1041_R1042insL mutation, a R1042_E1046dup, a E1043_R1044insK mutation, a R1044_I1045insM mutation, a R1044_I1045insR mutation, a I1045_E1046insD mutation, a E1046_F1047insRERIE mutation, a E1046_F1047insAAMRERIE mutation, a F1047delinsLL mutation, a L1048dup mutation, a L1048_N1049insL mutation, or a N1049delinsLD mutation.

The mutation at D555 may be a substitution or an insertion mutation. In some embodiments, the mutation is a substitution mutation at D555 of IGF1R. Exemplary substation mutations include, but are not limited to, a D555A mutation, a D555Y mutation, a D555E mutation, a D555N mutation, or a D555G mutation. In some embodiments, the mutation is an insertion mutation at D555 of IGF1R. For example, in some embodiments, the mutation is D555_L556insVD.

Certain hotspot mutations are particularly indicative of adenoid cystic carcinoma, and can be used to identify a cancer as an adenoid cystic carcinoma. In some embodiments, the cancer was previously identified as a cancer other than an adenoid cystic carcinoma. Thus, the mischaracterized cancer may be properly characterized as adenoid cystic carcinoma, based at least on the detection or the acquisition of knowledge of an IGF1R polypeptide having a mutation or a nucleic acid molecule encoding the IGF1R polypeptide having the mutation in a sample from the individual. The presence of the mutation in IGF1R indicates an increased likelihood that the cancer is an adenoid cystic carcinoma. The identification may be further based on additional clincopathological data. For example, the method may further include identifying a basaloid appearance, presence of cribriform architecture, or stroma characteristic of an adenoid cystic carcinoma, within a histological image of the cancer, which further indicates the cancer is an adenoid cystic carcinoma.

### (i) Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; assessing an IGF1R polypeptide having an activating mutation, or a nucleic acid molecule encoding an IGF1R polypeptide having an activating mutation, in a cancer in an individual; detecting the presence or absence of a cancer in an individual; monitoring progression or recurrence of a cancer in an individual; or identifying a candidate treatment for a cancer in an individual in need thereof.

In some embodiments, of any of the methods provided herein, the methods comprise acquiring knowledge of or detecting in a sample from an individual having a cancer an IGF1R polypeptide having an activating mutation, or a nucleic acid molecule encoding an IGF1R polypeptide having an activating mutation, described above and/or in the Examples herein. In other embodiments, the methods comprise acquiring knowledge of or detecting in a sample from an individual having a cancer an IGF1R polypeptide having an activating mutation, or a nucleic acid molecule encoding an IGF1R polypeptide having an activating mutation, of the disclosure, e.g., any of an IGF1R polypeptide having an activating mutation, or a nucleic acid molecule encoding an IGF1R polypeptide having an activating mutation, described above and/or in the Examples herein.

In some embodiments of any of the methods provided herein, detection of an IGF1R polypeptide having an activating mutation, or a nucleic acid molecule encoding an IGF1R polypeptide having an activating mutation, of the disclosure in the sample identifies the individual as one who may benefit from a treatment comprising the anti-cancer therapy, such as an anti-cancer therapy provided herein, e.g., an IGF1R-targeted therapy.

In some embodiments, the methods comprise detecting, in a first sample obtained from the individual at a first time point, the presence or absence of an IGF1R polypeptide having an activating mutation, or a nucleic acid molecule encoding an IGF1R polypeptide having an activating mutation, of the disclosure. In some embodiments, the methods further comprise detecting, in a second sample obtained from the individual at a second time point after the first time point, the presence or absence of an IGF1R polypeptide having an activating mutation, or a nucleic acid molecule encoding an IGF1R polypeptide having an activating mutation, of the disclosure. In some embodiments, the methods further comprise providing an assessment of cancer progression or cancer recurrence in the individual based, at least in part, on the presence or absence of the IGF1R polypeptide having the activating mutation, or the nucleic acid molecule encoding the IGF1R polypeptide having the activating mutation, in the first sample and/or in the second sample. In some embodiments, the presence of the IGF1R polypeptide having the activating mutation, or a nucleic acid molecule encoding the IGF1R polypeptide having the activating mutation, in the first sample and/or in the second sample identifies the individual as having increased risk of cancer progression or cancer recurrence. In some embodiments, the methods further comprise selecting a treatment, administering a treatment, adjusting a treatment, adjusting the dose of a treatment, or applying a treatment to the individual based, at least in part, on detecting the presence of the IGF1R polypeptide having the activating mutation, or a nucleic acid molecule encoding the IGF1R polypeptide having the activating mutation, in the first sample and/or in the second sample, wherein the treatment comprises an anti-cancer therapy, such as an anti-cancer therapy provided herein, e.g., and IGF1R-targeted therapy.

In some embodiments, the methods comprise performing DNA sequencing on a sample obtained from the individual to determine a sequencing mutation profile on a group of genes, wherein the sequencing mutation profile identifies the presence or absence of a nucleic acid molecule encoding an IGF1R polypeptide having an activating mutation of the disclosure. In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein, e.g., an IGF1R-targeted therapy. In some embodiments, the sequencing mutation profile identifies the presence or absence of a fragment of a nucleic acid molecule encoding an IGF1R polypeptide having an activating mutation, e.g., one or more of the corresponding activating mutations of IGF1R described herein. In some embodiments, the presence of a nucleic acid molecule encoding the IGF1R polypeptide having the activating mutation in the sample identifies the individual as one who may benefit from a treatment comprising an anti-cancer therapy, e.g., an anti-cancer therapy provided herein, such as an IGF1R-targeted therapy. In some embodiments, the presence of a nucleic acid molecule encoding the IGF1R polypeptide having the activating mutation in the sample predicts the individual to have longer survival when treated with a treatment comprising an anti-cancer therapy (e.g., an anti-cancer therapy provided herein, such as an IGF1R-targeted therapy), as compared to survival of an individual whose cancer does not comprise a nucleic acid molecule encoding the IGF1R polypeptide having the activating mutation.

In some embodiments of any of the methods provided herein, the methods further comprise generating a report comprising one or more treatment options identified for an individual (e.g., an individual having cancer) based at least in part on detection of the IGF1R polypeptide having the activating mutation, or a nucleic acid molecule encoding the IGF1R polypeptide having the activating mutation, of the disclosure in a sample from the individual, wherein the one or more treatment options comprise an anti-cancer therapy, such as an anti-cancer therapy provided herein, e.g., an IGF1R-targeted therapy.

In some embodiments of any of the methods provided herein, responsive to acquisition of knowledge of the IGF1R polypeptide having the activating mutation, or a nucleic acid molecule encoding the IGF1R polypeptide having the activating mutation of the disclosure in a sample from an individual (e.g., an individual having cancer): (i) the individual is classified as a candidate to receive a treatment comprising an anti-cancer therapy, such as an anti-cancer therapy provided herein, e.g., an IGF1R-targeted therapy; and/or (ii) the individual is identified as likely to respond to a treatment that comprises an anti-cancer therapy, such as an anti-cancer therapy provided herein, e.g., an IGF1R-targeted therapy. In some embodiments, responsive to acquisition of knowledge of the IGF1R polypeptide having the activating mutation, or a nucleic acid molecule encoding the IGF1R polypeptide having the activating mutation of the disclosure in a sample from an individual (e.g., an individual having cancer), the individual is predicted to have longer survival when treated with a treatment comprising an anti-cancer therapy, such as an anti-cancer therapy provided herein, e.g., an IGF1R-targeted therapy, as compared to survival of an individual whose cancer does not comprise or exhibit the IGF1R polypeptide having the activating mutation, or a nucleic acid molecule encoding the IGF1R polypeptide having the activating mutation. In some embodiments, responsive to acquisition of knowledge of the IGF1R polypeptide having the activating mutation, or a nucleic acid molecule encoding the IGF1R polypeptide having the activating mutation of the disclosure in a sample from an individual (e.g., an individual having cancer), the individual is predicted to have acquired resistance to a prior anti-cancer therapy administered to the individual, the individual is predicted to respond to an anti-cancer therapy (e.g., an anti-cancer therapy provided herein, such as an IGF1R-targeted therapy), and/or the individual is predicted to have poor prognosis, as compared to an individual whose cancer does not comprise the fusion nucleic acid molecule or polypeptide.

In some embodiments, responsive to acquisition of knowledge of the IGF1R polypeptide having the activating mutation, or a nucleic acid molecule encoding the IGF1R polypeptide having the activating mutation of the disclosure in a sample from an individual (e.g., an individual having cancer), the methods comprise administering to the individual an effective amount of a treatment that comprises an anti-cancer therapy, such as an anti-cancer therapy provided herein, e.g., an IGF1R-targeted therapy.

In some embodiments of any of the methods provided herein, the methods further comprise generating a report comprising one or more treatment options identified for an individual (e.g., an individual having cancer) based at least in part on knowledge of the presence of the IGF1R polypeptide having the activating mutation, or a nucleic acid molecule encoding the IGF1R polypeptide having the activating mutation of the disclosure in a sample from the individual, wherein the one or more treatment options comprise an anti-cancer therapy, such as an anti-cancer therapy provided herein, e.g., an IGF1R-targeted therapy.

In some embodiments, acquiring knowledge of the IGF1R polypeptide having the activating mutation, or a nucleic acid molecule encoding the IGF1R polypeptide having an activating mutation, of the disclosure in a sample comprises detecting the fusion nucleic acid molecule or polypeptide in the sample.

In some embodiments of any of the methods provided herein, a nucleic acid molecule encoding the IGF1R polypeptide having an activating mutation the disclosure comprises detecting a fragment of the fusion nucleic acid molecule comprising an activating mutation described above and/or in the Examples herein.

In some embodiments of any of the methods provided herein, detecting an IGF1R polypeptide having and activating mutation of the disclosure comprises detecting a portion of the IGF1R polypeptide having an activating mutation, e.g., one or more of the corresponding activating mutations described above and/or in the Examples herein.

In some embodiments, the methods further comprise providing an assessment of the IGF1R polypeptide having the activating mutation, or a nucleic acid molecule encoding the IGF1R polypeptide having the activating mutation of the disclosure.

In some embodiments of any of the methods provided herein, the anti-cancer therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising the IGF1R polypeptide having an activating mutation, or a nucleic acid molecule encoding the IGF1R polypeptide having an activating mutation, a treatment for cancer being tested in a clinical trial, a targeted therapy, a treatment being tested in a clinical trial for cancer comprising an IGF1R polypeptide having an activating mutation, or a nucleic acid molecule encoding the IGF1R polypeptide having an activating mutation, or any combination thereof, e.g., a described in further detail below. In some embodiments, the anti-cancer therapy is a kinase inhibitor, such as a kinase inhibitor described herein or known in the art. In some embodiments, the kinase inhibitor is a multi-kinase inhibitor or an IGF1R-specific inhibitor known in the art or described herein. In some embodiments, the kinase inhibitor is a tyrosine kinase inhibitor. In some embodiments, the kinase inhibitor inhibits the kinase activity of an IGF1R polypeptide. In some embodiments, the anti-cancer therapy is an IGR1-targeted therapy, such as any of cixutumumab, figitumumab, dalotuzumab, ganitumab, robatumumab, BMS-754807, NVP-ADW742, NVP-AEW541, OSI-906, teprotumumab, ceritinib, xentuzumab, AXL1717, IGF-MTX, W0101, FPI-1434, SCH717454, AVE1642, BIIB022, or MEDI-573, linsitinib, ceritinib, BMS-754807, BVP 51004, XL228, or INSM-18, and any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of the IGF1R polypeptide having an activating mutation, or a nucleic acid molecule encoding the IGF1R polypeptide having an activating mutation of the disclosure. In some embodiments, the nucleic acid comprises a double-stranded RNA (dsRNA), a small interfering RNA (siRNA), or a small hairpin RNA (shRNA), e.g., as described herein.

In some embodiments of any of the methods provided herein, the methods further comprise acquiring knowledge of or detecting in a sample from the individual a base substitution, a short insertion/deletion (delins), a copy number alteration, or a genomic rearrangement in one or more genes other than IGF1R. In some embodiments, the one or more genes comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, or ZNF703 gene, or any combination thereof. In some embodiments, the one or more genes comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods provided herein, the treatment or the one or more treatment options further comprise an additional anti-cancer therapy. In some embodiments of any of the methods provided herein, the treatment or the one or more treatment options further comprise administering an additional anti-cancer therapy to the individual. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, the individual has been previously treated, or is being treated, for cancer with a treatment for cancer, e.g., an anti-cancer therapy described herein or any other anti-cancer therapy or treatment known in the art. In some embodiments, the IGF1R polypeptide having the activating mutation, or a nucleic acid molecule encoding the IGF1R polypeptide having an activating mutation, of the disclosure confers resistance of the cancer to the prior or current treatment for cancer.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, multiple myeloma, bone marrow multiple myeloma, melanoma, breast cancer, lung cancer, bronchus cancer, colorectal cancer, prostate cancer, pancreatic cancer, stomach cancer, ovarian cancer, ovary epithelial carcinoma, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, testis germ cell tumor, biliary tract cancer, small bowel cancer, appendix cancer, salivary gland cancer, thyroid gland cancer, adrenal gland cancer, osteosarcoma, soft tissue osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL) or bone marrow leukemia lymphocytic acute (ALL), bone marrow leukemia T cell acute (T-ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, squamous cell cancer, non-squamous cell cancer, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, lung adenocarcinoma, non-small cell lung cancer, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, peritoneum adenocarcinoma, soft tissue malignant peripheral nerve sheath tumor (MPNST) or a carcinoid tumor.

In some embodiments, the cancer is acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some embodiments, the cancer is a breast cancer, a head and neck cancer, an adenoid cystic carcinoma, an anal cancer, a bladder cancer, a uterine cancer, a lung cancer, a skin cancer, a neuroendocrine cancer, an ovarian cancer, or a prostate cancer. In some embodiments, the cancer is an adenoid cystic carcinoma. In some embodiments, the cancer is a basaloid cancer. In some embodiments, the cancer is a head or neck cancer, such as a salivary gland cancer. In some embodiments, the cancer is a basaloid salivary gland cancer. In some embodiments, the cancer is a salivary gland adenoid cystic carcinoma.

In some embodiments, the cancer is a metastatic cancer. In some embodiments, the cancer is a primary cancer.

In some embodiments, the methods of the disclosure further comprise detecting the presence or absence of a cancer in a sample from the individual. In some embodiments, the methods further comprise administering an effective amount of anti-cancer therapy to the individual, e.g., an anti-cancer therapy described herein, e.g., an IGF1R-targeted therapy.

In some embodiments, any of the cancers described herein comprise any of the IGF1R polypeptides having an activating mutation, or a nucleic acid molecule encoding any of the IGF1R polypeptides having an activating mutation, of the disclosure, e.g., an IGF1R activating mutation described above and/or in the Examples herein.

In some embodiments, the methods provided herein comprise acquiring knowledge of or detecting any of the nucleic acid molecules encoding the IGF1R polypeptide having an activating mutation of the disclosure, e.g., an IGF1R activating mutation described above and/or in the Examples herein, in a sample from an individual having any cancer known in the art, or any of the cancers described herein. In some embodiments, the methods provided herein comprise acquiring knowledge of or detecting any of the IGF1R polypeptides of the disclosure, e.g., an IGF1R polypeptide having an activating mutation described above and/or in the Examples herein, in a sample from an individual having any cancer known in the art, or any of the cancers described herein.

In some embodiments, a cancer provided in any of Tables 2-4 in Example 1 herein comprises the corresponding IGF1R polypeptide having the activating mutation, or nucleic acid molecule encoding the IGF1R polypeptide having the activating mutation, as described in any of Table 2-4.

In some embodiments, the cancer is negative for a MYB, MYBL1, or NOTCH1 driver mutation. In some embodiments, the cancer is negative for a MYB-NFIB fusion.

### B. Detection of IGF1R Nucleic Acid Molecules and Polypeptides Having Activating Mutations

Certain aspects of the present disclosure relate to detection of a nucleic acid molecule encoding an IGF1R polypeptide having an activating mutation of the disclosure, e.g., in a sample from an individual (e.g., a patient). In some embodiments, the nucleic acid molecule encoding an IGF1R polypeptide having an activating mutation is detected in vitro.

Other aspects of the present disclosure relate to detection of an IGF1R polypeptide having an activating mutation of the disclosure, e.g., in a sample from an individual (e.g., a patient). In some embodiments, the IGF1R polypeptide having the mutation is detected in vitro.

### (i) Detection of IGF1R Nucleic Acid Molecules

Methods for detecting a nucleic acid molecule encoding an IGF1R polypeptide having an activating mutation of the disclosure are known in the art. For example, in some embodiments, a nucleic acid molecule encoding an IGF1R polypeptide having an activating mutation of the disclosure is detected by sequencing part or all of the IGF1R gene or expressed transcript thereof, for example by next-generation or other sequencing of DNA, RNA, or cDNA. In some embodiments, the sequencing is a targeted sequencing, for example by targeting the IGF1R gene or expressed transcript thereof, or a portion of the IGF1R gene or expressed transcript thereof (e.g., a portion containing a hotspot region described herein).

In some embodiments, a nucleic acid molecule encoding an IGF1R polypeptide having an activating mutation of the disclosure is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting a nucleic acid molecule encoding an IGF1R polypeptide having an activating mutation of the disclosure are provided below.

In some embodiments, a nucleic acid molecule encoding an IGF1R polypeptide having an activating mutation of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, a nucleic acid molecule encoding an IGF1R polypeptide having an activating mutation of the disclosure is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, a nucleic acid molecule encoding an IGF1R polypeptide having an activating mutation of the disclosure is detected by PCR amplification of DNA, RNA, or cDNA.

In some embodiments, a nucleic acid molecule encoding an IGF1R polypeptide having an activating mutation of the disclosure is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of a mutant nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, a nucleic acid molecule encoding an IGF1R polypeptide having an activating mutation of the disclosure is detected using a sequencing method. Any method of sequencing known in the art can be used to detect a nucleic acid molecule encoding an IGF1R polypeptide having an activating mutation provided herein. Exemplary sequencing methods that may be used to detect a mutant nucleic acid molecule provided herein include those based on techniques developed by Maxam and Gilbert or Sanger. Automated sequencing procedures may also be used, e.g., including sequencing by mass spectrometry.

In some embodiments, a nucleic acid molecule encoding an IGF1R polypeptide having an activating mutation of the disclosure is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, a nucleic acid molecule encoding an IGF1R polypeptide having an activating mutation of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10′ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a nucleic acid molecule encoding an IGF1R polypeptide having an activating mutation provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect a mutant nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual suspected of having or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, sample index sequences, or unique molecular identifier (UMI) sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual suspected of having or determined to have cancer), wherein the plurality of nucleic acid molecules comprises nucleic acid molecules corresponding to a nucleic acid molecule encoding an IGF1R polypeptide having an activating mutation of the disclosure; (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the nucleic acid molecule encoding the IGF1R polypeptide having the activating mutation; (f) analyzing the plurality of sequence reads; and (g) based on the analysis, detecting the presence or absence of the nucleic acid molecule encoding the IGF1R polypeptide having the activating mutation in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the IGF1R polypeptide having the activating mutation. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a nucleic acid molecule encoding an IGF1R polypeptide having an activating mutation of the disclosure in said library to produce an enriched sample; (e) sequencing the enriched sample, thereby producing a plurality of sequence reads; (f) analyzing the plurality of sequence reads for the presence of the nucleic acid molecule encoding the IGF1R polypeptide having the activating mutation; (g) detecting, based on the analyzing step, the presence or absence of the nucleic acid molecule encoding the IGF1R polypeptide having the activating mutation in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods, the one or more adapters comprise amplification primers, flow cell adaptor sequences, substrate adapter sequences, sample index sequences, or unique molecular identifier (UMI) sequences. In some embodiments, the one or more adapters comprise one or more sample index sequences. As is known in the art, sample indexes allow the sequencing of multiple samples on the same instrument flow cell or chip (i.e., multiplexing). Sample indexes are typically between about 8 and about 10 bases in length, and comprise a nucleotide sequence specific to a sample that is used to assign sequence reads to the correct sample during data analysis. In some embodiments, the one or more adapters comprise one or more unique molecule identifiers (UMIs). As is known in the art, UMIs comprise short nucleotide sequences that include a unique barcode that is incorporated into each molecule in a given sample library. UMIs are useful for identifying PCR duplicates created during library amplification steps, and/or for reducing the rate of false-positive variant calls and increasing variant detection, since variant alleles present in the original sample (true variants) can be distinguished from errors introduced during library preparation, target enrichment, or sequencing.

In some embodiments, the selectively enriching comprises: (a) combining one or more bait molecules with the library, thereby hybridizing the one or more bait molecules to one or more nucleic acid molecules comprising nucleotide sequences corresponding to the nucleic acid molecule encoding the IGF1R polypeptide having the activating mutation and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to a nucleic acid molecule encoding an IGF1R polypeptide having an activating mutation of the disclosure. In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the nucleic acid molecule encoding the IGF1R polypeptide having the activating mutation and producing nucleic acid hybrids; and (b) isolating the nucleic acid hybrids to produce the enriched sample. In some embodiments, the selectively enriching comprises amplifying the one or more nucleic acids comprising nucleotide sequences corresponding to the nucleic acid molecule encoding the IGF1R polypeptide having the activating mutation using a polymerase chain reaction (PCR) to produce an enriched sample. In some embodiments, the methods further comprise sequencing the enriched sample.

In some embodiments of any of the methods provided herein, the methods further comprise analyzing sequence data (e.g., obtained from sequencing as described above), for the presence or absence of one or more alterations (e.g., a base substitution, a short insertion/deletion (delins), a copy number alteration, or a genomic rearrangement) in one or more genes (e.g., one or more cancer-related genes, or a panel of known/suspected oncogenes and/or tumor suppressors, or any combination thereof). In some embodiments, the presence or absence of one or more gene alterations of the disclosure is detected using any suitable method known in the art, e.g., as described in Frampton et al., (2013) Nat Biotechnol, 31:1023-1031. In some embodiments, base substitution alterations are detected using Bayesian methodology, which allows detection of novel somatic mutations at low mutant allele frequency (MAF) and increased sensitivity for mutations at hotspot sites through the incorporation of tissue-specific prior expectations. See, e.g., Kim et al., Cancer Discov (2011) 1:44-53 and Frampton et al., (2013) Nat Biotechnol, 31:1023-1031. In some embodiments, insertion/deletion (delins) alterations are detected using any suitable method, such as de novo local assembly, e.g., using the de Bruijn approach, see, e.g., Compeau et al., Nat Biotechnol (2011) 29:987-991 and Frampton et al., (2013) Nat Biotechnol, 31:1023-1031. In some embodiments, gene fusion and genomic rearrangement alterations are detected using any suitable method, such as by analyzing chimeric read pairs (read pairs for which reads map to separate chromosomes, or at a distance of over 10 Mbp), see, e.g., Frampton et al., (2013) Nat Biotechnol, 31:1023-1031. In some embodiments, rearrangements are annotated for predicted function (e.g., creation of fusion gene or tumor suppressor inactivation). In some embodiments, the one or more genes comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof. In some embodiments, the one or more genes comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods provided herein, the methods further comprise generating a molecular profile for the individual or the sample, based, at least in part, on detecting the presence or absence of a nucleic acid molecule encoding an IGF1R polypeptide having an activating mutation of the disclosure. In some embodiments, the molecular profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some instances, a molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some embodiments of any of the methods provided herein, the methods further comprise selecting a treatment, administering a treatment, or applying a treatment to the individual based on the generated molecular profile, wherein the treatment comprises an anti-cancer therapy, e.g., as described herein, e.g., an IGF1R-targeted therapy. In some embodiments of any of the methods provided herein, the methods further comprise generating a report indicating the presence or absence of a nucleic acid molecule encoding an IGF1R polypeptide having an activating mutation of the disclosure in the sample. In some embodiments of any of the methods provided herein, the methods further comprise generating, by one or more processors, a report indicating the presence or absence of a nucleic acid molecule encoding an IGF1R polypeptide having an activating mutation of the disclosure in the sample. In some embodiments, the report comprises the generated molecular profile. In some embodiments, the methods further comprise providing or transmitting the report, e.g., as described below. In some embodiments, the report is transmitted via a computer network or a peer-to-peer connection. In some instances, all or a portion of the report may be displayed in a graphical user interface of an online or web-based healthcare portal.

In some embodiments of any of the methods provided herein, the methods for determining the presence or absence of a nucleic acid molecule encoding an IGF1R polypeptide having an activating mutation may be implemented as part of a genomic profiling process that comprises identification of the presence of variant sequences at one or more gene loci (e.g., one or more gene loci as listed above) in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci (e.g., one or more gene loci as listed above). In some instances, the variant panel selected for genomic profiling may comprise detection of variant sequences at a number of gene loci (e.g., one or more gene loci as listed above) through comprehensive genomic profiling (CGP), a next-generation sequencing (NGS) approach used to assess hundreds of genes (including relevant cancer biomarkers) in a single assay. Inclusion of the disclosed methods for determining the presence or absence of a nucleic acid molecule encoding an IGF1R polypeptide having an activating mutation as part of a genomic profiling process can improve the validity of, e.g., disease detection calls by, for example, independently confirming the presence of the nucleic acid molecule encoding the IGF1R polypeptide having an activating mutation in a given patient sample.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample.

In some embodiments of any of the methods provided herein, the method further comprises determining the circulating tumor DNA (ctDNA) fraction of a liquid biopsy sample.

### (ii) Detection of IGF1R Polypeptides

Also provided herein are methods of detecting an IGF1R polypeptide having an activating mutation of the disclosure, or a mutation-containing fragment thereof.

An IGF1R polypeptide having an activating mutation provided herein, or a mutation-containing fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments an IGF1R polypeptide having an activating mutation provided herein, or a mutation-containing fragment thereof, can be distinguished from a reference polypeptide, e.g., a non-mutant or wild type protein or polypeptide, with an antibody or antibody fragment that reacts differentially with a mutant protein or polypeptide (e.g., an IGF1R polypeptide having an activating mutation provided herein or a mutation-containing fragment thereof) as compared to a reference protein or polypeptide. In some embodiments, an IGF1R polypeptide having an activating mutation of the disclosure, or a mutation-containing fragment thereof, can be distinguished from a reference polypeptide, e.g., a non-mutant or wild type protein or polypeptide, by reaction with a detection reagent, e.g., a substrate, e.g., a substrate for catalytic activity, e.g., phosphorylation.

In some aspects, methods of detection of an IGF1R polypeptide having an activating mutation of the disclosure (e.g., an IGF1R polypeptide having an activating mutation encoded by an IGF1R nucleic acid molecule of the disclosure), or a mutation-containing fragment thereof, are provided, comprising contacting a sample, e.g., a sample described herein, comprising an IGF1R polypeptide having an activating mutation described herein, with a detection reagent provided herein (e.g., an antibody of the disclosure), and determining if the IGF1R polypeptide having the activating mutation polypeptide is present in the sample.

(iii) Detection Reagents

In some aspects, provided herein are reagents for detecting an IGF1R polypeptide having an activating mutation of the disclosure, or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises a nucleic acid molecule encoding an IGF1R polypeptide having an activating mutation described herein, or a mutation-containing fragment or portion thereof.

In other aspects, provided herein are reagents for detecting an IGF1R polypeptide having an activating mutation of the disclosure, or a fragment thereof, e.g., an IGF1R polypeptide having an activating mutation encoded by an IGF1R nucleic acid molecule of the disclosure, or a fragment thereof, e.g., according to the methods of detection provided herein. In some embodiments, a detection reagent provided herein comprises an antibody or antibody fragment that specifically binds to an IGF1R polypeptide having an activating mutation of the disclosure, or to a mutation-containing fragment thereof.

### Baits

In some embodiments, nucleic acids corresponding to IGF1R are captured (e.g., from amplified nucleic acids) by hybridization with a bait molecule. Provided herein are bait molecules suitable for the detection a nucleic acid molecule encoding an IGF1R polypeptide having an activating mutation of the disclosure.

In some embodiments, a bait molecule comprises a capture nucleic acid molecule configured to hybridize to a target nucleic acid molecule comprising a nucleic acid molecule encoding an IGF1R polypeptide having an activating mutation of the disclosure, or a fragment or portion thereof. In some embodiments, the capture nucleic acid molecule is configured to hybridize to the nucleic acid molecule encoding the IGF1R polypeptide having the activating mutation of the target nucleic acid molecule.

In some embodiments, the capture nucleic acid molecule is configured to hybridize to a fragment of the nucleic acid molecule encoding the IGF1R polypeptide having an activating mutation acid molecule of the disclosure. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises the activating mutation of IGF1R of the disclosure.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a hotspot region of the nucleic acid molecule encoding an IGF1R polypeptide having an activating mutation of the disclosure, and/or hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the hotpot region. In some embodiments, the capture nucleic acid molecule is configured to hybridize to a nucleotide sequence in an intron or an exon of an IGF1R gene, or in a hotspot region for an activating mutation of the IGF1R gene, as described herein. In some embodiments, the capture nucleic acid molecule is configured to hybridize to exon 9 of the IGF1R gene, and/or to a region flanking exon 9 of the IGF1R gene such as within 10, within 20, within 30, within 40, within 50, within 60, within 70, within 80, within 90, or within 100 nucleotides of exon 9 of the IGF1R gene. In some embodiments, the capture nucleic acid molecule is configured to hybridize to exon 8 of the IGF1R gene (which includes the D555 hotspot locus), and/or to a region flanking exon 8 of the IGF1R gene such as within 10, within 20, within 30, within 40, within 50, within 60, within 70, within 80, within 90, or within 100 nucleotides of exon 8 of the IGF1R gene. In some embodiments, the capture nucleic acid molecule is configured to hybridize to exon 16 of the IGF1R gene (which includes kinase domain hotspot region), and/or to a region flanking exon 16 of the IGF1R gene such as within 10, within 20, within 30, within 40, within 50, within 60, within 70, within 80, within 90, or within 100 nucleotides of exon 16 of the IGF1R gene.

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having an activating mutation of IGF1R described herein, from a reference nucleotide sequence, e.g., a nucleotide sequence not having the activating mutation.

### Probes

Also provided herein are probes, e.g., nucleic acid molecules, suitable for the detection of a nucleic acid molecule encoding an IGF1R polypeptide having an activating mutation of the disclosure. In some embodiments, a probe provided herein comprises a nucleic acid sequence configured to hybridize to a target nucleic acid molecule that is or comprises a nucleic acid molecule encoding an IGF1R polypeptide having an activating mutation of the disclosure, or a mutation-containing fragment or portion thereof. In some embodiments, the probe comprises a nucleic acid sequence configured to hybridize to the nucleic acid molecule encoding an IGF1R polypeptide having an activating mutation of the disclosure, or the fragment or portion thereof, of the target nucleic acid molecule. In some embodiments, the probe comprises a nucleic acid sequence configured to hybridize to a mutation-containing fragment or portion of the nucleic acid molecule encoding an IGF1R polypeptide having an activating mutation of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a hotspot region or activating mutation of a nucleic acid molecule encoding a IGF1R polypeptide having the activating mutation of the disclosure, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the mutation or hotspot. In some embodiments, the probe is configured to hybridize to exon 9 of the IGF1R gene, and/or to a region flanking exon 9 of the IGF1R gene such as within 10, within 20, within 30, within 40, within 50, within 60, within 70, within 80, within 90, or within 100 nucleotides of exon 9 of the IGF1R gene. In some embodiments, the probe is configured to hybridize to exon 8 of the IGF1R gene (which includes the D555 hotspot locus), and/or to a region flanking exon 8 of the IGF1R gene such as within 10, within 20, within 30, within 40, within 50, within 60, within 70, within 80, within 90, or within 100 nucleotides of exon 8 of the IGF1R gene. In some embodiments, the probe is configured to hybridize to exon 16 of the IGF1R gene (which includes kinase domain hotspot region), and/or to a region flanking exon 16 of the IGF1R gene such as within 10, within 20, within 30, within 40, within 50, within 60, within 70, within 80, within 90, or within 100 nucleotides of exon 16 of the IGF1R gene.

In some embodiments, the probe comprises a nucleic acid molecule, which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detecting a nucleic acid molecule encoding an IGF1R polypeptide having an activating mutation provided herein. For example, a probe provided herein may be used for detecting a nucleic acid molecule encoding an IGF1R polypeptide having an activating mutation of the disclosure in a sample, e.g., a sample obtained from an individual. In some embodiments, the probe may be used for identifying cells or tissues that express a nucleic acid molecule encoding an IGF1R polypeptide having an activating mutation of the disclosure, e.g., by measuring levels of the nucleic acid molecule encoding an IGF1R polypeptide having an activating mutation. In some embodiments, the probe may be used for detecting levels of a nucleic acid molecule encoding an IGF1R polypeptide having an activating mutation of the disclosure, e.g., mRNA levels, in a sample of cells from an individual.

In some embodiments, a probe provided herein specifically hybridizes to a nucleic acid molecule comprising a rearrangement (e.g., a deletion, inversion, insertion, duplication, or other rearrangement) resulting in a nucleic acid molecule encoding an IGF1R polypeptide having an activating mutation of the disclosure. In some embodiments, a probe provided herein specifically hybridizes to a nucleic acid molecule comprising a substitution/missense mutation resulting in a nucleic acid molecule encoding an IGF1R polypeptide having an activating mutation of the disclosure.

In some embodiments, a probe of the disclosure distinguishes a nucleic acid, e.g., a genomic or transcribed nucleic acid, e.g., a cDNA or RNA, having an activating mutation in a nucleic acid molecule encoding an IGF1R polypeptide from a reference nucleotide sequence, e.g., a nucleotide sequence not having the activating mutation.

Also provided herein are isolated pairs of allele-specific probes, wherein, for example, the first probe of the pair specifically hybridizes to in a nucleic acid molecule encoding an IGF1R polypeptide having an activating mutation of the disclosure, and the second probe of the pair specifically hybridizes to a corresponding wild type sequence (e.g., a wild type IGF1R nucleic acid molecule). Probe pairs can be designed and produced for any of the IGF1R nucleic acid molecules described herein and are useful in detecting a somatic mutation in a sample. In some embodiments, a first probe of a pair specifically hybridizes to a mutation (e.g., duplication, deletion, insertion, delins, or missense/substitution resulting in a nucleic acid molecule encoding an IGF1R polypeptide having an activating mutation described herein), and a second probe of a pair specifically hybridizes to a sequence upstream or downstream of the mutation.

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises a nucleic acid molecule encoding an IGF1R polypeptide having an activating mutation of the disclosure, or a mutation-containing fragment or portion thereof. In some embodiments, the oligonucleotide comprises a nucleotide sequence configured to hybridize to the nucleic acid molecule encoding the IGF1R polypeptide having the activating mutation of the target nucleic acid molecule. In some embodiments, the oligonucleotide comprises a nucleotide sequence configured to hybridize to a mutation-containing fragment or portion of the nucleic acid molecule encoding the IGF1R polypeptide having the activating mutation of the target nucleic acid molecule.

In some embodiments, the oligonucleotide, e.g., the primer, comprises a nucleotide sequence configured to hybridize to an activating mutation or a hotspot region of a nucleic acid molecule encoding an IGF1R polypeptide of the disclosure, and may be further configured to hybridize to between about 10 and about 12, about 12 and about 15, about 15 and about 17, about 17 and about 20, about 20 and about 25, or about 25 and about 30, or more nucleotides flanking either side of the mutation or hotspot region. In some embodiments, the oligonucleotide, e.g., the primer, is configured to hybridize to exon 9 of the IGF1R gene, and/or to a region flanking exon 9 of the IGF1R gene such as within 10, within 20, within 30, within 40, within 50, within 60, within 70, within 80, within 90, or within 100 nucleotides of exon 9 of the IGF1R gene. In some embodiments, the oligonucleotide, e.g., the primer, is configured to hybridize to exon 8 of the IGF1R gene (which includes the D555 hotspot locus), and/or to a region flanking exon 8 of the IGF1R gene such as within 10, within 20, within 30, within 40, within 50, within 60, within 70, within 80, within 90, or within 100 nucleotides of exon 8 of the IGF1R gene. In some embodiments, the oligonucleotide, e.g., the primer, is configured to hybridize to exon 16 of the IGF1R gene (which includes kinase domain hotspot region), and/or to a region flanking exon 16 of the IGF1R gene such as within 10, within 20, within 30, within 40, within 50, within 60, within 70, within 80, within 90, or within 100 nucleotides of exon 16 of the IGF1R gene.

In some embodiments, the oligonucleotide comprises a nucleotide sequence corresponding to a nucleic acid molecule encoding the IGF1R polypeptide having the activating mutation of the disclosure. In some embodiments, the oligonucleotide comprises a nucleotide sequence corresponding to a mutation-containing fragment or a portion of the nucleic acid molecule encoding the IGF1R polypeptide having the activating mutation. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a nucleic acid molecule encoding the IGF1R polypeptide having an activating mutation provided herein. In some embodiments, the oligonucleotide comprises a nucleotide sequence complementary to a fragment or a portion of the nucleic acid molecule encoding the IGF1R polypeptide having the activating mutation provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises a nucleic acid molecule encoding an IGF1R polypeptide having an activating mutation of the disclosure, or a fragment thereof, e.g., using PCR. In some embodiments, the oligonucleotide may be used to sequence a nucleic acid molecule that is or comprises a nucleic acid molecule that is or comprises a nucleic acid molecule encoding an IGF1R polypeptide having an activating mutation provided herein, or a fragment thereof. In some embodiments, the oligonucleotide may be used to amplify a nucleic acid molecule comprising an activating mutation of a nucleic acid molecule that is or comprises a nucleic acid molecule encoding an IGF1R polypeptide having an activating mutation described herein, e.g., using PCR. In some embodiments, the oligonucleotide may be used to sequence a nucleic acid molecule comprising an activating mutation in a nucleic acid molecule encoding an IGF1R polypeptide or fragment thereof.

In some embodiments, pairs of oligonucleotides, e.g., pairs of primers, are provided herein, which are configured to hybridize to a nucleic acid molecule that is or comprises a nucleic acid molecule encoding an IGF1R polypeptide having an activating mutation of the disclosure, or a mutation-containing fragment thereof. In some embodiments, a pair of oligonucleotides of the disclosure may be used for directing amplification of a nucleic acid molecule encoding an IGF1R polypeptide having an activating mutation or mutation-containing fragment thereof, e.g., using a PCR reaction. In some embodiments, pairs of oligonucleotides, e.g., pairs of primers, are provided herein, which are configured to hybridize to a nucleic acid molecule comprising an activating mutation of an IGF1R nucleic acid molecule described herein, e.g., for use in directing amplification of the corresponding IGF1R nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a nucleic acid molecule encoding an IGF1R polypeptide having an activating mutation of the disclosure, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having an activating mutation of an IGF1R nucleic acid molecule described herein from a reference nucleotide sequence, e.g., a nucleotide sequence not having the activating mutation.

In one aspect, provided herein is a primer or primer set for amplifying a nucleic acid molecule comprising a cytogenetic abnormality such as an insertion, delins, substitution or other mutation resulting in a nucleic acid molecule encoding an IGF1R polypeptide having an activating mutation of the disclosure. In another aspect, provided herein is a primer or primer set for amplifying a nucleic acid molecule comprising an insertion, delins, substitution or other mutation resulting in a nucleic acid molecule encoding an IGF1R polypeptide having an activating mutation of the disclosure. In certain aspects, provided herein are allele-specific oligonucleotides, e.g., primers, wherein a first oligonucleotide of a pair specifically hybridizes to an activating mutation, and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., an activating mutation of an IGF1R nucleic acid molecule described herein), and a second oligonucleotide of the pair specifically hybridizes to a sequence downstream of the mutation.

In some embodiments, the oligonucleotide, e.g., the primer, hybridizes to an activating mutation or hotspot region of an IGF1R nucleic acid molecule described herein and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the mutation or hotspot region).

### Antibodies

Provided herein are antibodies or antibody fragments that specifically bind to an IGF1R polypeptide having an activating mutation of the disclosure, or a mutation-containing fragment thereof, e.g., an IGF1R polypeptide having an activating mutation encoded by an IGF1R nucleic acid molecule of the disclosure, or a fragment thereof.

The antibody or antibody fragment may be of any suitable type of antibody or antibody fragment, including, but not limited to, a monoclonal, polyclonal, or multispecific (e.g., a bispecific) antibody or antibody fragment, so long as the antibody or antibody fragment exhibits a specific antigen binding activity, e.g., binding to an IGF1R polypeptide having an activating mutation of the disclosure, or a fragment thereof.

In some embodiments, an IGF1R polypeptide having an activating mutation of the disclosure, or a fragment thereof, is used as an immunogen to generate one or more antibodies or antibody fragments of the disclosure, e.g., using standard techniques for polyclonal and monoclonal antibody preparation. In some embodiments, an IGF1R polypeptide having an activating mutation provided herein, is used to provide antigenic peptide fragments (e.g., comprising any of at least about 8, at least about 10, at least about 15, at least about 20, at least about 30 or more amino acids) for use as immunogens to generate one or more antibodies or antibody fragments of the disclosure, e.g., using standard techniques for polyclonal and monoclonal antibody preparation. As is known in the art, an antibody or antibody fragment of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., an IGF1R polypeptide having an activating mutation of the disclosure, or a mutation-containing fragment thereof. The preparation can further include an adjuvant, such as Freund's complete or incomplete adjuvant, or a similar immunostimulatory agent.

In some embodiments, an antibody or antibody fragment provided herein is a polyclonal antibody. Methods of producing polyclonal antibodies and fragments thereof are known in the art. In some embodiments, an antibody or antibody fragment provided herein is a monoclonal antibody, wherein a population of the antibody or fragment molecules contain only one species of an antigen binding site capable of immunoreacting or binding with a particular epitope, e.g., an epitope on an IGF1R polypeptide having an activating mutation provided herein. Methods of preparation of monoclonal antibodies and fragments thereof are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody or antibody fragment of the disclosure may also be identified and isolated by screening a recombinant combinatorial immunoglobulin library (e.g., an antibody phage display library) with the polypeptide of interest, e.g., an IGF1R polypeptide having an activating mutation provided herein or a mutation-containing fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies or antibody fragments of the disclosure are recombinant, such as chimeric or humanized monoclonal antibodies or antibody fragments, comprising both human and non-human portions. Such chimeric and/or humanized monoclonal antibodies or antibody fragments can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody or antibody fragment of the disclosure is a human monoclonal antibody or antibody fragment. In some embodiments, human monoclonal antibodies or antibody fragments are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies or fragments thereof, and protocols for producing such antibodies or fragments thereof, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody or antibody fragment of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody or antibody fragment of the disclosure can be used to isolate an IGF1R polypeptide having an activating mutation provided herein, or a fragment thereof, by standard techniques, such as affinity chromatography or immunoprecipitation. In some embodiments, an antibody or antibody fragment of the disclosure can be used to detect an IGF1R polypeptide having an activating mutation provided herein, or a mutation-containing fragment thereof, e.g., in a tissue sample, cellular lysate, or cell supernatant, in order to evaluate the level and/or pattern of expression of the IGF1R polypeptide having an activating mutation. Detection can be facilitated by coupling the antibody or antibody fragment to a detectable substance. Thus, in some embodiments, an antibody or antibody fragment of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, D galactosidase-, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of an IGF1R polypeptide having an activating mutation provided herein) in tissues or body fluids (e.g., in a tumor cell-containing tissue or body fluid), e.g., according to the methods provided herein.

In certain embodiments, an antibody or antibody fragment provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) for its target, e.g., an IGF1R polypeptide having an activating mutation of the disclosure. Methods of measuring antibody or antibody fragment affinity (e.g., Kd) are known in the art, and include, without limitation, a radiolabeled antigen-binding assay (RIA) and a BIACORE® surface plasmon resonance (SPR) assay. In some embodiments, antibody affinity (e.g., Kd) is determined using the Fab version of an antibody of the disclosure and its antigen (e.g., an IGF1R polypeptide having an activating mutation provided herein), e.g., using RIA or SPR.

In certain embodiments, an antibody fragment provided herein is a Fab, Fab′, Fab′-SH, F(ab′)₂, Fv, single-chain antibody molecule (e.g., scFv), scFv-Fc fragment, and other fragments described herein or known in the art. In certain embodiments, an antibody fragment provided herein is a diabody. Diabodies are antibody fragments with two antigen-binding sites that may be bivalent or bispecific. In certain embodiments, an antibody fragment provided herein is a triabody or a tetrabody. In certain embodiments, an antibody fragment provided herein is a single-domain antibody fragment. Single-domain antibody fragments comprise all or a portion of the heavy chain variable domain or all or a portion of the light chain variable domain of an antibody. In certain embodiments, a single-domain antibody fragment is a human single-domain antibody fragment.

Antibody fragments can be made by various techniques, including but not limited to proteolytic digestion of an intact antibody, as well as production by recombinant host cells (e.g., E. coli or phage), as known in the art and as described herein.

In certain embodiments, an antibody or antibody fragment provided herein is a chimeric antibody. In one example, a chimeric antibody or antibody fragment comprises a non-human variable region (e.g., a variable region derived from a mouse, rat, hamster, rabbit, or non-human primate, such as a monkey), and a human constant region or portions thereof. In a further example, a chimeric antibody or antibody fragment is a “class switched” antibody or antibody fragment, in which the class or subclass of the antibody or antibody fragment has been changed from that of the parent antibody or antibody fragment. Chimeric antibodies include antigen-binding fragments thereof.

In certain embodiments, a chimeric antibody or antibody fragment is a humanized antibody or antibody fragment. Typically, a non-human antibody or antibody fragment is humanized to reduce immunogenicity to humans, while retaining the specificity and affinity of the parental non-human antibody or antibody fragment. Generally, a humanized antibody or antibody fragment comprises one or more variable domains in which HVRs, e.g., CDRs, (or portions thereof), are derived from a non-human antibody, and framework regions (FRs) (or portions thereof) are derived from human antibody sequences. A humanized antibody or antibody fragment optionally will also comprise at least a portion of a human constant region. In some embodiments, some FR residues in a humanized antibody or antibody fragment are substituted with corresponding residues from a non-human antibody or antibody fragment (e.g., the antibody or antibody fragment from which the HVR residues are derived), e.g., to restore or improve specificity or affinity. Humanized antibodies or antibody fragments, and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody or antibody fragment provided herein is a human antibody. Human antibodies or antibody fragments can be produced using various techniques known in the art. For example, human antibodies or antibody fragments may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies or antibody fragments can also be made by hybridoma-based methods known in the art, e.g., using known human myeloma and mouse-human heteromyeloma cell lines for the production of human monoclonal antibodies. Human antibodies or antibody fragments may also be generated by isolating Fv clone variable domain sequences selected from human-derived phage display libraries. Such variable domain sequences may then be combined with a desired human constant domain or portions thereof. Techniques for selecting human antibodies or fragments thereof from libraries are known in the art and described herein.

Antibodies or antibody fragments of the disclosure may be isolated by screening combinatorial libraries for antibodies or fragments thereof with the desired activity or activities. For example, a variety of methods are known in the art for generating phage display libraries and screening such libraries for antibodies or antibody fragments possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies or fragments thereof to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody or antibody fragment provided herein is multispecific, e.g., bispecific. Multispecific antibodies or antibody fragments are monoclonal antibodies or antibody fragments that have binding specificities for at least two different sites or at least two different antigens. For example, one of the binding specificities can be to an IGF1R polypeptide having an activating mutation of the disclosure, and the other can be to any other antigen. Techniques for making multispecific antibodies or antibody fragments are known in the art and include, but are not limited to, recombinant co-expression of two immunoglobulin heavy chain-light chain pairs having different specificities, and “knob-in-hole” engineering. Multispecific antibodies or antibody fragments may also be made by engineering electrostatic steering effects (e.g., by introducing mutations in the constant region) for making heterodimeric Fcs; cross-linking two or more antibodies or fragments; using leucine zippers to produce bispecific antibodies or antibody fragments; using “diabody” technology for making bispecific antibody fragments; using single-chain Fv (scFv) dimers; and preparing trispecific antibodies or antibody fragments. Engineered antibodies or antibody fragments with three or more functional antigen binding sites, including “Octopus antibodies,” are also included in the disclosure. Antibodies or antibody fragments of the disclosure also include “Dual Acting FAbs” or “DAF,” e.g., comprising an antigen-binding site that binds to an IGF1R polypeptide having an activating mutation of the disclosure as well as another, different antigen.

In certain embodiments, amino acid sequence variants of the antibodies or antibody fragments provided herein are contemplated. For example, it may be desirable to improve the binding affinity and/or other biological properties of the antibody or antibody fragment. Amino acid sequence variants of an antibody or antibody fragment of the disclosure may be prepared by introducing appropriate modifications into the nucleotide sequence encoding the antibody or antibody fragment, or by peptide synthesis. Such modifications include, for example, deletions, and/or insertions, and/or substitutions of residues within the amino acid sequences of the antibody or antibody fragment. Any combination of deletions, insertions, and substitutions can be made to arrive at the final antibody or antibody fragment, provided that the final antibody or antibody fragment possesses the desired characteristics, e.g., antigen-binding.

In certain embodiments, antibody or antibody fragment variants having one or more amino acid substitutions are provided. Sites of interest for substitutional mutagenesis include the HVRs and FRs. Amino acid substitutions may be introduced into an antibody or antibody fragment of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody or antibody fragment of the present disclosure is altered to increase or to decrease the extent to which the antibody is glycosylated. Addition or deletion of glycosylation sites to an antibody or antibody fragment may be conveniently accomplished by altering the amino acid sequence of the antibody or antibody fragment, such that one or more glycosylation sites is created or removed. Antibody or antibody fragment variants having bisected oligosaccharides are further provided, e.g., in which a biantennary oligosaccharide attached to the Fc region of the antibody or antibody fragment is bisected by GlcNAc. In some embodiments, antibody or antibody fragment variants of the disclosure may have increased fucosylation. In some embodiments, antibody or antibody fragment variants of the disclosure may have reduced fucosylation. In some embodiments, antibody or antibody fragment variants of the disclosure may have improved ADCC function. In some embodiments, antibody or antibody fragment variants of the disclosure may have decreased ADCC function. Antibody or antibody fragment variants with at least one galactose residue in the oligosaccharide attached to the Fc region are also provided. Such variants may have improved CDC function. In some embodiments, antibody or antibody fragment variants of the disclosure may have increased or decreased CDC function.

In certain embodiments, one or more amino acid modifications may be introduced into the Fc region of an antibody or antibody fragment of the present disclosure, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the present disclosure contemplates an antibody or antibody fragment variant that possesses some but not all effector functions, which make it a desirable candidate for applications in which the half-life of the antibody or antibody fragment in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or antibody fragment lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies or antibody fragments with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody or antibody fragment variants with improved or diminished binding to FcRs are also included in the disclosure. In certain embodiments, an antibody or antibody fragment variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) C1q binding and/or CDC. In some embodiments, antibodies or antibody fragments of the disclosure have increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody or antibody fragment provided herein is cysteine-engineered, e.g., “thioMAb,” in which one or more residues of the antibody or antibody fragment are substituted with cysteine residues. In some embodiments, the substituted residues occur at accessible sites of the antibody or antibody fragment. By substituting those residues with cysteine, reactive thiol groups are thereby positioned at accessible sites of the antibody or antibody fragment, and may be used to conjugate the antibody or antibody fragment to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies or antibody fragments may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody or antibody fragment, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody or antibody fragment may vary, and if more than one polymer is attached, the polymers can be the same or different molecules. In general, the number and/or type of polymers used for derivatization can be determined based on considerations including, but not limited to, the particular properties or functions of the antibody or antibody fragment to be improved, or whether the antibody or antibody fragment derivative will be used in a therapy under defined conditions. In some embodiments, provided herein are antibodies or antibody fragments conjugated to carbon nanotubes, e.g., for use in methods to selectively heat the antibody or antibody fragment using radiation to a temperature at which cells proximal to the antibody or antibody fragment are killed.

### (iv) Samples

A variety of materials can be the source of, or serve as, samples for use in any of the methods of the disclosure, such as the methods for detection of an IGF1R polypeptide having an activating mutation, or a nucleic acid molecule encoding the IGF1R polypeptide having an activating mutation of the disclosure, or fragments thereof (e.g., mutation-containing fragments thereof).

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics, and the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, a sample refers to a preparation that is obtained by processing (e.g., by removing one or more components of and/or by adding one or more agents to) a primary sample. Such a processed sample may comprise, for example, nucleic acids (e.g., for use in any of the methods for detection of a nucleic acid molecule encoding the IGF1R polypeptide having an activating mutation provided herein) or proteins (e.g., for use in any of the methods for detection of IGF1R polypeptides having an activating mutation provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises a nucleic acid molecule that encodes an IGF1R polypeptide having an activating mutation, or an IGF1R polypeptide having an activating mutation, of the disclosure, or fragments or portions thereof. In some embodiments, the methods provided herein comprise obtaining one or more samples from the individual (e.g., the individual having a cancer). In some embodiments, the one or more samples are obtained or derived from a cancer (e.g., a cancer in an individual). In some embodiments, the one or more samples comprise at least 20% tumor cell nuclear area.

In some embodiments, the sample is a control sample or a reference sample, e.g., not containing an IGF1R polypeptide having an activating mutation, or a nucleic acid molecule encoding the IGF1R polypeptide having an activating mutation described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the IGF1R polypeptides having an activating mutation, or nucleic acid molecules encoding the IGF1R polypeptide having an activating mutation, described herein. In other embodiments, the reference sample is from a non-tumor or cancer sample, e.g., a blood control, a normal adjacent tumor (NAT), or any other non-cancerous sample from the same or a different individual.

In some embodiments, a nucleic acid molecule encoding the IGF1R polypeptide having an activating mutation provided herein is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, a nucleic acid molecule encoding the IGF1R polypeptide having an activating mutation of the disclosure is detected in a sample comprising cell-free DNA (cfDNA), cell-free RNA, and/or circulating tumor DNA (ctDNA). In some embodiments, a nucleic acid molecule encoding the IGF1R polypeptide having an activating mutation of the disclosure is detected in a sample comprising cell-free DNA (cfDNA) and/or circulating tumor DNA (ctDNA). In some embodiments, a nucleic acid molecule encoding the IGF1R polypeptide having an activating mutation of the disclosure is detected in a sample comprising circulating tumor DNA (ctDNA).

In some embodiments, any of the methods of the present disclosure comprise acquiring knowledge of or detecting any of the biomarkers described herein (e.g., an IGF1R polypeptide having an activating mutation, or a nucleic acid molecule encoding the IGF1R polypeptide having an activating mutation) in one or more samples (e.g., as described above) obtained from an individual (e.g., an individual having a cancer). In some embodiments, the samples used to acquire knowledge of or detect any of the biomarkers described herein (e.g., an IGF1R polypeptide having an activating mutation, or a nucleic acid molecule encoding the IGF1R polypeptide having an activating mutation, and/or one or more alterations in one or more genes) are the same sample (i.e., one or more, or all, of an IGF1R polypeptide having an activating mutation, or a nucleic acid molecule encoding the IGF1R polypeptide having an activating mutation, and one or more alterations in one or more genes are detected or determined in one sample). In some embodiments, the samples used to acquire knowledge of or detect any of the biomarkers described herein (e.g., an IGF1R polypeptide having an activating mutation, or a nucleic acid molecule encoding the IGF1R polypeptide having an activating mutation, and/or one or more alterations in one or more genes) comprise more than one sample (e.g., some of the biomarkers may be detected or determined in one sample, and some of the biomarkers may be detected or determined in another sample). For example, in some embodiments an IGF1R polypeptide having an activating mutation, or a nucleic acid molecule encoding the IGF1R polypeptide having an activating mutation, may be detected in one sample, and one or more alterations in one or more genes may be detected or determined in another sample. In another example, a nucleic acid molecule encoding an IGF1R polypeptide having an activating mutation may be detected in one sample, and an IGF1R polypeptide having an activating mutation may be detected or determined in another sample.

### C. Anti-Cancer Therapies

Certain aspects of the present disclosure relate to anti-cancer therapies, as well as methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering a treatment comprising an anti-cancer therapy or applying a treatment comprising an anti-cancer therapy to an individual based on a generated molecular and/or sequencing mutation profile. An anti-cancer therapy can refer to an agent or a compound that is effective in the treatment of cancer cells. Examples of anti-cancer agents, anti-cancer compounds, or anti-cancer therapies include, but are not limited to, alkylating agents, antimetabolites, natural products, hormones, chemotherapy, radiation therapy, immunotherapy, surgery, or a therapy configured to target a defect in a specific cell-signaling pathway, e.g., a defect in a DNA mismatch repair (MMR) pathway.

In some embodiments, an anti-cancer therapy of the disclosure is a small molecule inhibitor, an antibody (e.g., a monoclonal antibody), a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an IGF1R polypeptide having an activating mutation, or a nucleic acid molecule encoding the IGF1R polypeptide having an activating mutation, of the disclosure, a treatment for cancer being tested in a clinical trial, a targeted therapy, a treatment being tested in a clinical trial for cancer comprising an IGF1R polypeptide having an activating mutation, or a nucleic acid molecule encoding the IGF1R polypeptide having an activating mutation, of the disclosure, or any combination thereof, e.g., a described in further detail below. In some embodiments, the anti-cancer therapy is an IGF1R-targeted therapy. In some embodiments, the anti-cancer therapy, e.g., the IGF1R-targeted therapy is a kinase inhibitor, such as a kinase inhibitor described herein or known in the art. In some embodiments, the kinase inhibitor is a tyrosine kinase inhibitor. In some embodiments, the kinase inhibitor inhibits the kinase activity of an IGF1R polypeptide. In some embodiments, the kinase inhibitor is a multi-kinase inhibitor or an IGF1R-specific inhibitor known in the art or described herein. In some embodiments, the nucleic acid inhibits the expression of an IGF1R polypeptide having an activating mutation, or a nucleic acid molecule encoding the IGF1R polypeptide having an activating mutation of the disclosure.

In some embodiments, the anti-cancer therapy, e.g., the IGF1R-targeted therapy comprises cixutumumab, figitumumab, dalotuzumab, ganitumab, robatumumab, BMS-754807, NVP-ADW742, NVP-AEW541, OSI-906, teprotumumab, ceritinib, xentuzumab, AXL1717, IGF-MTX, WO101, FPI-1434, SCH717454, AVE1642, BIIB022, or MEDI-573, linsitinib, BMS-754807, BVP 51004, XL228, or INSM-1 or any combination thereof.

In some embodiments, an anti-cancer therapy of the disclosure, e.g., an IGF1R-targeted therapy is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, an anti-cancer therapy of the disclosure comprises a cyclin-dependent kinase (CDK) inhibitor, e.g., alone or in combination with an IGF1R-targeted therapy. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with an IGF1R-targeted therapy. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with an IGF1R-targeted therapy) one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor, e.g., alone or in combination with an IGF1R-targeted therapy. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with an IGF1R-targeted therapy. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, RO5126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with an IGF1R-targeted therapy. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with an IGF1R-targeted therapy. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with an IGF1R-targeted therapy. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with an IGF1R-targeted therapy.

In some embodiments, the anti-cancer therapy comprises one or more of an immune checkpoint inhibitor, a chemotherapy, a VEGF inhibitor, an Integrin 33 inhibitor, a statin, an EGFR inhibitor, an mTOR inhibitor, a PI3K inhibitor, a MAPK inhibitor, or a CDK4/6 inhibitor, e.g., alone or in combination with an IGF1R-targeted therapy.

In some embodiments, the anti-cancer therapy comprises a kinase inhibitor, e.g., alone or in combination with an IGF1R-targeted therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor, e.g., alone or in combination with an IGF1R-targeted therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with an IGF1R-targeted therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with an IGF1R-targeted therapy. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CiA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor, e.g., alone or in combination with an IGF1R-targeted therapy. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin 33 inhibitor, e.g., alone or in combination with an IGF1R-targeted therapy. In some embodiments, the integrin 33 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with an IGF1R-targeted therapy. In some embodiments, the αIIbβ3 integrin inhibitor is abciximab, eptifibatide (e.g., Integrilin®), or tirofiban (e.g., Aggrastat®).

In some embodiments, the anti-cancer therapy comprises an mTOR inhibitor, e.g., alone or in combination with an IGF1R-targeted therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with an IGF1R-targeted therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with an IGF1R-targeted therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor, e.g., alone or in combination with an IGF1R-targeted therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (ACO0010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., alone or in combination with an IGF1R-targeted therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to an IGF1R polypeptide having an activating mutation of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with an IGF1R-targeted therapy. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody-based vaccine, or a cell-based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid-based vaccine in which the nucleic acid encodes the polypeptides. In some embodiments, a cancer vaccine is formulated as an antibody-based vaccine. In some embodiments, a cancer vaccine is formulated as a cell-based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21, 2013). In some embodiments, such cancer vaccines augment the anti-cancer response.

In some embodiments, the cancer vaccine comprises a polynucleotide that encodes a neoantigen, e.g., a neoantigen corresponding to an IGF1R polypeptide having a mutation or a nucleic acid molecule encoding the IGF1R polypeptide having the mutation of the disclosure. In some embodiments, the cancer vaccine comprises DNA that encodes a neoantigen, e.g., a neoantigen corresponding to an IGF1R polypeptide having a mutation or a nucleic acid molecule encoding the IGF1R polypeptide having the mutation of the disclosure. In some embodiments, the cancer vaccine comprises RNA that encodes a neoantigen, e.g., a neoantigen corresponding to an IGF1R polypeptide having a mutation or a nucleic acid molecule encoding the IGF1R polypeptide having the mutation of the disclosure. In some embodiments, the cancer vaccine comprises a polynucleotide that encodes a neoantigen, e.g., a neoantigen corresponding to an IGF1R polypeptide having a mutation or a nucleic acid molecule encoding the IGF1R polypeptide having the mutation of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g., human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to an IGF1R polypeptide having a mutation or a nucleic acid molecule encoding the IGF1R polypeptide having the mutation of the disclosure.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to an IGF1R polypeptide having a mutation or a nucleic acid molecule encoding the IGF1R polypeptide having the mutation of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a nucleic acid molecule that encodes an IGF1R polypeptide having an activating mutation, or an IGF1R polypeptide having an activating mutation, of the disclosure, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen corresponding to an IGF1R polypeptide having a mutation or a nucleic acid molecule encoding the IGF1R polypeptide having the mutation of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g., via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor, e.g., alone or in combination with an IGF1R-targeted therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSFI4, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis-binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, e.g., Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (I1BI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine, e.g., alone or in combination with an IGF1R-targeted therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-30, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid that encodes an IGF1R polypeptide having an activating mutation of the disclosure or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a fusion nucleic acid molecule of the disclosure or a portion thereof comprising a breakpoint. Methods for designing, optimizing, producing, and using dsRNAs, siRNAs, or shRNAs, are known in the art.

In some embodiments, the anti-cancer therapy comprises a chemotherapy, e.g., alone or in combination with an IGF1R-targeted therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor, e.g., alone or in combination with an IGF1R-targeted therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises any of abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aligopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubega), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane I131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof, e.g., alone or in combination with an IGF1R-targeted therapy.

In some embodiments, the anti-cancer therapy comprises an anti-angiogenic agent, e.g., alone or in combination with an IGF1R-targeted therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina νβ3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet-derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy, e.g., alone or in combination with an IGF1R-targeted therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer, e.g., alone or in combination with an IGF1R-targeted therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent, e.g., alone or in combination with an IGFR1-targeted therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNP, IFNγ, IFN-γ inducing factor (IGIF); transforming growth factor-β (TGF-0); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, the anti-cancer therapy comprises an anti-hormonal agent, e.g., alone or in combination with an IGFR1-targeted therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent, e.g., alone or in combination with an IGFR1-targeted therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODA™), 6-mercaptopurine, methotrexate, 6-thioguanine, pemetrexed, raltitrexed, arabinosylcytosine ARA-C cytarabine (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent, e.g., alone or in combination with an IGFR1-targeted therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 119, Marcel Dekker, 2002.

Formulations to be used for in vivo administration are sterile. This is readily accomplished by filtration through sterile filtration membranes or other methods known in the art.

In some embodiments, an anti-cancer therapy of the disclosure is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

### D. Reporting

In some embodiments, the methods provided herein comprise generating a report, and/or providing a report to party.

In some embodiments, a report according to the present disclosure comprises information about one or more of: an IGF1R polypeptide having an activating mutation, or a nucleic acid molecule encoding an IGF1R polypeptide having an activating mutation, of the disclosure; a cancer of the disclosure, e.g., comprising an IGF1R polypeptide having an activating mutation, or a nucleic acid molecule encoding an IGF1R polypeptide having an activating mutation of the disclosure; or a treatment, a therapy, or one or more treatment options for an individual having a cancer, such as a cancer of the disclosure (e.g., an IGF1R polypeptide having an activating mutation, or a nucleic acid molecule encoding an IGF1R polypeptide having an activating mutation described herein).

In some embodiments, a report according to the present disclosure comprises information about the presence or absence of an IGF1R polypeptide having an activating mutation, or a nucleic acid molecule encoding an IGF1R polypeptide having an activating mutation, of the disclosure in a sample obtained from an individual, such as an individual having a cancer, e.g., a cancer provided herein. In one embodiment, a report according to the present disclosure indicates that an IGF1R polypeptide having an activating mutation, or a nucleic acid molecule encoding an IGF1R polypeptide having an activating mutation, of the disclosure is present in a sample obtained from the individual. In one embodiment, a report according to the present disclosure indicates that an IGF1R polypeptide having an activating mutation, or a nucleic acid molecule encoding an IGF1R polypeptide having an activating mutation, of the disclosure is not present in a sample obtained from the individual. In one embodiment, a report according to the present disclosure indicates that an IGF1R polypeptide having an activating mutation, or a nucleic acid molecule encoding an IGF1R polypeptide having an activating mutation, of the disclosure has been detected in a sample obtained from the individual. In one embodiment, a report according to the present disclosure indicates that an IGF1R polypeptide having an activating mutation, or a nucleic acid molecule encoding an IGF1R polypeptide having an activating mutation, of the disclosure has not been detected in a sample obtained from the individual. In some embodiments, the report comprises an identifier for the individual from which the sample was obtained.

In some embodiments, the report includes information on the role of an IGF1R polypeptide having an activating mutation, or a nucleic acid molecule encoding an IGF1R polypeptide having an activating mutation, of the disclosure, or its wild type counterparts, in disease, such as in cancer. Such information can include one or more of: information on prognosis of a cancer, such as a cancer provided herein, e.g., comprising an IGF1R polypeptide having an activating mutation, or a nucleic acid molecule encoding an IGF1R polypeptide having an activating mutation, described herein; information on resistance of a cancer, such as a cancer provided herein, e.g., comprising an IGF1R polypeptide having an activating mutation, or a nucleic acid molecule encoding an IGF1R polypeptide having an activating mutation, described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising an IGF1R polypeptide having an activating mutation, or a nucleic acid molecule encoding an IGF1R polypeptide having an activating mutation, described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting an IGF1R polypeptide having an activating mutation, or a nucleic acid molecule encoding an IGF1R polypeptide having an activating mutation, of the disclosure in the sample, or acquiring knowledge of the presence of an IGF1R polypeptide having an activating mutation, or a nucleic acid molecule encoding an IGF1R polypeptide having an activating mutation, of the disclosure in the sample; and generating a report. In some embodiments, a report generated according to the methods provided herein comprises one or more of: information about the presence or absence of an IGF1R polypeptide having an activating mutation, or a nucleic acid molecule encoding an IGF1R polypeptide having an activating mutation, of the disclosure in the sample; an identifier for the individual from which the sample was obtained; information on the role of an IGF1R polypeptide having an activating mutation, or a nucleic acid molecule encoding an IGF1R polypeptide having an activating mutation, of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising an IGF1R polypeptide having an activating mutation, or a nucleic acid molecule encoding an IGF1R polypeptide having an activating mutation of the disclosure), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting an IGF1R polypeptide having an activating mutation, or a nucleic acid molecule encoding an IGF1R polypeptide having an activating mutation of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence an IGF1R polypeptide having an activating mutation, or a nucleic acid molecule encoding an IGF1R polypeptide having an activating mutation of the disclosure in a sample obtained from an individual (e.g., an individual having a cancer). In some instances, all or a portion of the report may be displayed in a graphical user interface of an online or web-based healthcare portal.

### E. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform A method according to any of the embodiments described herein.

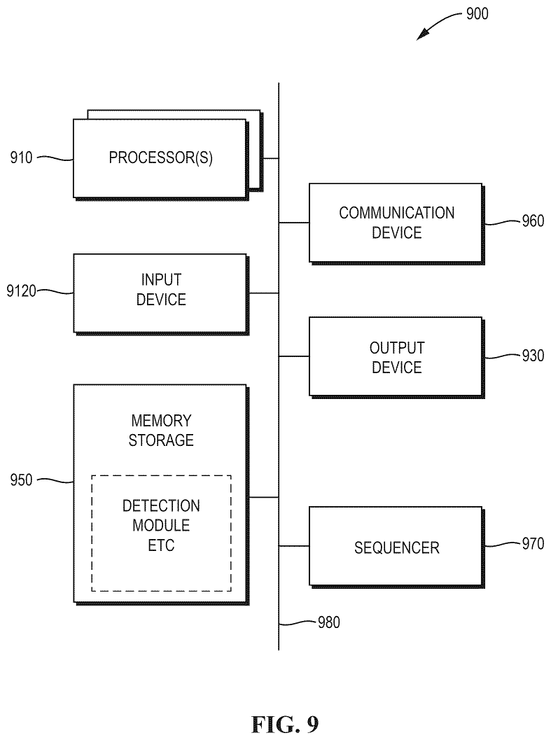

FIG. 9 illustrates an example of a computing device or system in accordance with one embodiment. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting an IGF1R polypeptide having an activating mutation, or a nucleic acid molecule encoding an IGF1R polypeptide having an activating mutation of the disclosure. Input device 920 and output device 930 can generally correspond to those described herein, and can be either connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting an a nucleic acid molecule that encodes an IGF1R polypeptide having an activating mutation of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

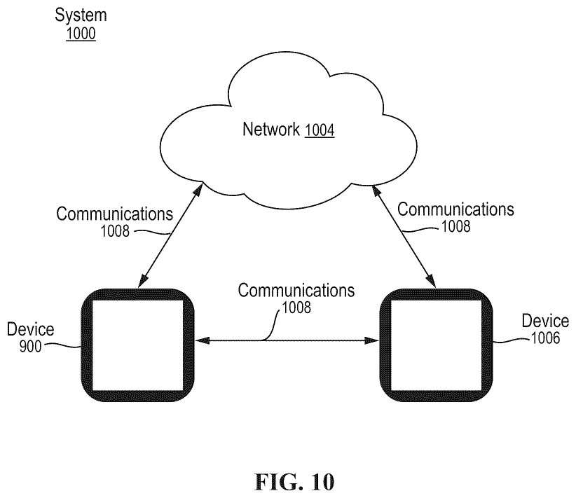

Device 900 may be connected to a network (e.g., network 1004, as shown in FIG. 10 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

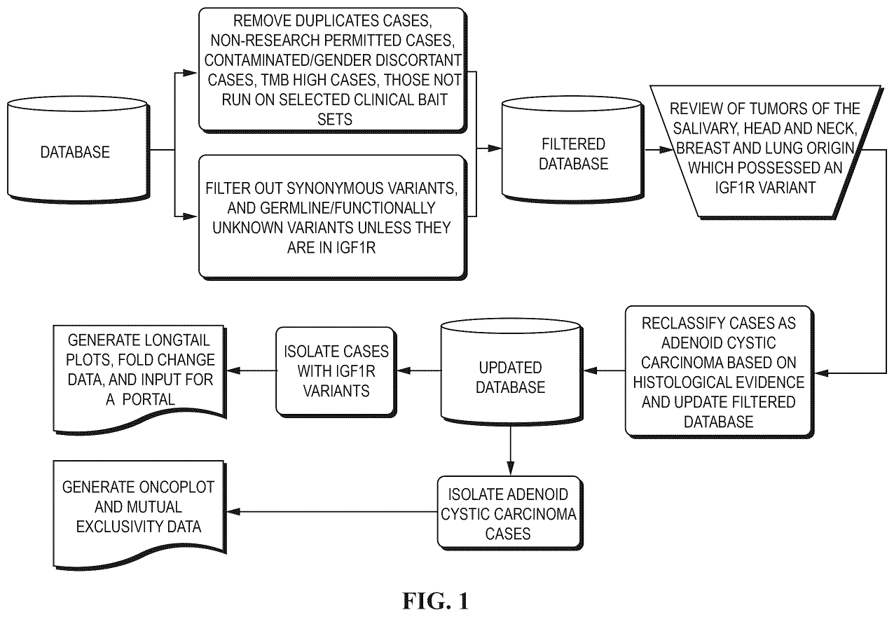

FIG. 10 illustrates an example of a computing system in accordance with one embodiment. In computing system 1000, device 900 (e.g., as described above and illustrated in FIG. 1) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

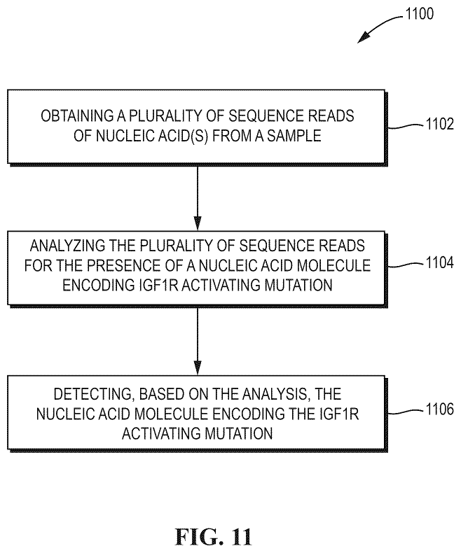

FIG. 11 illustrates an exemplary process 1100 for detecting an IGF1R polypeptide having an activating mutation, or a nucleic acid molecule encoding an IGF1R polypeptide having an activating mutation, of the disclosure in a sample, in accordance with some embodiments of the present disclosure. Process 1100 is performed, for example, using one or more electronic devices implementing a software program. In some examples, process 1100 is performed using a client-server system, and the blocks of process 1100 are divided up in any manner between the server and a client device. In other examples, the blocks of process 1100 are divided up between the server and multiple client devices. Thus, while portions of process 1100 are described herein as being performed by particular devices of a client-server system, it will be appreciated that process 1100 is not so limited. In some embodiments, the executed steps can be executed across many systems, e.g., in a cloud environment. In other examples, process 1100 is performed using only a client device or only multiple client devices. In process 1100, some blocks are, optionally, combined, the order of some blocks is, optionally, changed, and some blocks are, optionally, omitted. In some examples, additional steps may be performed in combination with the process 1100. Accordingly, the operations as illustrated (and described in greater detail below) are exemplary by nature and, as such, should not be viewed as limiting.

At block 1102, a plurality of sequence reads of one or more nucleic acid molecules is obtained, wherein the one or more nucleic acid molecules are derived from a sample obtained from an individual, e.g., as described herein. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: a nucleic acid molecule encoding an IGF1R polypeptide having an activating mutation, or fragments thereof. Optionally, prior to obtaining the sequence reads, the sample is purified, enriched (e.g., for nucleic acid(s) corresponding to: a nucleic acid molecule encoding an IGF1R polypeptide having an activating mutation or fragments thereof), and/or subjected to PCR amplification. At block 1104, an exemplary system (e.g., one or more electronic devices) analyzes the plurality of sequence reads for the presence of a nucleic acid molecule encoding an IGF1R polypeptide having an activating mutation of the disclosure, or a fragment thereof. At block 1106, the system detects (e.g., based on the analysis) a nucleic acid molecule encoding an IGF1R polypeptide having an activating mutation of the disclosure, or a fragment thereof, in the sample.

In some embodiments of any of the methods, systems, devices, non-transitory computer readable storage media, or processes of the disclosure, the nucleic acid molecule encoding an IGF1R polypeptide having an activating mutation is any of the nucleic acid molecules encoding an IGF1R polypeptide having an activating mutation described herein (e.g., as described above, for example, and/or in Example 1 or 2 herein). In some embodiments of any of the methods, systems, devices, non-transitory computer readable storage media, or processes of the disclosure, the IGF1R polypeptide having an activating mutation is any of the IGF1R polypeptide having an activating mutation described herein (e.g., as described above, for example; and/or in Example 1 or 2 herein). In some embodiments, the cancer is any cancer known in the art or described herein (e.g., as described above, for example; and/or in Example 1 herein). In some embodiments, any of the cancers described herein (e.g., as described above, for example, and/or in Example 1 or 2 herein) may comprise any of the IGF1R polypeptides having an activating mutation, or nucleic acid molecules encoding an IGF1R polypeptide having an activating mutation of the disclosure (e.g., as described above, for example; and/or in Example 1 or 2 herein). In some embodiments, a cancer provided in one of Tables 2-4 in Example 1 herein comprises the corresponding an IGF1R polypeptide having an activating mutation, or a nucleic acid molecule encoding an IGF1R polypeptide having an activating mutation as described in Table 2-4.

In some embodiments of any of the methods, systems, devices, non-transitory computer readable storage media, or processes of the disclosure, detection of an IGF1R polypeptide having an activating mutation, or a nucleic acid molecule encoding an IGF1R polypeptide having an activating mutation of the disclosure, in a cancer (e.g., in one or more samples) identifies the individual having the cancer as one who may benefit from a treatment comprising an anti-cancer therapy, e.g., an anti-cancer therapy provided herein, such as an IGF1R-targeted therapy. In some embodiments of any of the methods, systems, devices, non-transitory computer readable storage media, or processes of the disclosure, detection of an IGF1R polypeptide having an activating mutation, or a nucleic acid molecule encoding an IGF1R polypeptide having an activating mutation of the disclosure in a cancer (e.g., in one or more samples) predicts the individual having the cancer to have longer survival when treated with a treatment comprising an anti-cancer therapy, e.g., an IGF1R-targeted therapy, as compared to survival of an individual whose cancer does not comprise an IGF1R polypeptide having an activating mutation, or a nucleic acid molecule encoding an IGF1R polypeptide having an activating mutation. In some embodiments of any of the methods, systems, devices, non-transitory computer readable storage media, or processes of the disclosure, detection of an IGF1R polypeptide having an activating mutation, or a nucleic acid molecule encoding an IGF1R polypeptide having an activating mutation de of the disclosure in a cancer (e.g., in one or more samples) identifies the individual having the cancer to be a candidate to receive a treatment comprising an anti-cancer therapy, e.g., an IGF1R-targeted therapy. In some embodiments of any of the methods, systems, devices, non-transitory computer readable storage media, or processes of the disclosure, detection an IGF1R polypeptide having an activating mutation, or a nucleic acid molecule encoding an IGF1R polypeptide having an activating mutation of the disclosure in a cancer (e.g., in one or more samples) identifies the individual having the cancer as likely to respond (e.g., to have a therapeutic response) to a treatment comprising an anti-cancer therapy, e.g., an IGF1R-targeted therapy. In some embodiments of any of the methods, systems, devices, non-transitory computer readable storage media, or processes of the disclosure, detection of an IGF1R polypeptide having an activating mutation, or a nucleic acid molecule encoding an IGF1R polypeptide having an activating mutation of the disclosure in a cancer (e.g., in one or more samples) identifies the individual having the cancer as likely to have an improved response when treated with a treatment comprising an anti-cancer therapy, e.g., an IGF1R-targeted therapy, as compared to an individual whose cancer does not comprise an IGF1R polypeptide having an activating mutation, or a nucleic acid molecule encoding an IGF1R polypeptide having an activating mutation.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the individual is administered a treatment based at least in part on detection of an IGF1R polypeptide having an activating mutation, or a nucleic acid molecule encoding an IGF1R polypeptide having an activating mutation of the disclosure in the cancer in an individual (e.g., in one or more samples from the individual). In some embodiments, the treatment is an anti-cancer therapy known in the art or described herein, e.g., an IGF1R-targeted therapy.

In some embodiments of any of the methods, systems, devices, non-transitory computer readable storage media, or processes of the disclosure, the disclosed methods for determining the presence or absence of a nucleic acid molecule encoding an IGF1R polypeptide having an activating mutation of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a nucleic acid molecule encoding an IGF1R polypeptide having an activating mutation of the disclosure as part of a genomic profiling process can improve the validity of, e.g., disease detection calls, made on the basis of the genomic profiling by, for example, independently confirming the presence of the nucleic acid molecule encoding an IGF1R polypeptide having an activating mutation of the disclosure in a given patient sample. In some embodiments, the one or more gene loci comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene locus, or any combination thereof. In some embodiments, the one or more gene loci comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

In some instances, the comprehensive genomic profiling may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, the comprehensive genomic profiling may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some embodiments of any of the methods, systems, devices, non-transitory computer readable storage media, or processes of the disclosure, a molecular profile for a sample or for an individual is generated based at least in part on detecting a nucleic acid molecule encoding an IGF1R polypeptide having an activating mutation of the disclosure, or a fragment thereof (e.g., a mutation-containing fragment), in a sample. In some instances, the molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors. In some instances, the molecular profile may comprise results from a comprehensive genomic profiling (CGP) test (e.g., as describe above), a nucleic acid sequencing-based test, a gene expression profiling test, a cancer hotspot panel test, a DNA methylation test, a DNA fragmentation test, an RNA fragmentation test, or any combination thereof. In some embodiments, the molecular profile further comprises/indicates/comprises information on presence or absence of mutations in one or more additional genes, e.g., a panel of known/suspected oncogenes and/or tumor suppressors. In some embodiments, the molecular profile is obtained from a genomic profiling assay (such as a cancer- or tumor-related genomic profiling assay), e.g., as obtained using any of the sequencing methodologies described herein. In some embodiments, the molecular profile includes information from whole-genome or whole-exome sequencing. In some embodiments, the molecular profile includes information from targeted sequencing. In some embodiments, the molecular profile includes information from NGS. In some embodiments, the molecular profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known/suspected oncogenes and/or tumor suppressors, one or more cancer-related genes, or any combination thereof. In some embodiments, the individual is administered a treatment based at least in part on the molecular profile. In some embodiments, the treatment is an anti-cancer therapy known in the art or described herein. In some embodiments, the one or more genes comprise the ABL1, ACVR1B, AKT1, AKT2, AKT3, ALK, ALOX12B, AMER1, APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EMSY (C11orf30), EP300, EPHA3, EPHB1, EPHB4, ERBB2, ERBB3, ERBB4, ERCC4, ERG, ERRFIl, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCD1LG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, or ZNF703 gene, or any combination thereof. In some embodiments, the one or more genes comprise the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene, or any combination thereof.

In some embodiments of any of the methods, systems, devices, non-transitory computer readable storage media, or processes of the disclosure, a report is generated, e.g., as described in further detail above. In some embodiments, the report comprises/indicates/comprises information on the presence or absence of an IGF1R polypeptide having an activating mutation, or a nucleic acid molecule encoding an IGF1R polypeptide having an activating mutation, the disclosure in the cancer in an individual (e.g., in one or more samples from the individual). In some embodiments, the report comprises/indicates/comprises information on results of a genomic profiling process of a cancer in an individual (e.g., in one or more samples from the individual), e.g., as described above. In some embodiments, the report comprises/indicates/comprises information on results of comprehensive genomic profiling of a cancer in an individual (e.g., in one or more samples from the individual), e.g., as described above. In some embodiments, the report comprises/indicates/comprises information on a molecular profile generated for the individual or the sample, e.g., as described above. In some embodiments, the report comprises/indicates/comprises information on a treatment or one or more treatment options selected or identified for the individual, based, at least in part, on the presence of an IGF1R polypeptide having an activating mutation, or a nucleic acid molecule encoding an IGF1R polypeptide having an activating mutation, of the disclosure in the cancer in an individual (e.g., in one or more samples from the individual), and optionally based on results of a genomic profiling process, comprehensive genomic profiling, and/or a molecular profile generated for the individual or a sample, e.g., as described above. In some embodiments, the treatment or one or more treatment options comprise an anti-cancer therapy known in the art or described herein, e.g., an IGF1R-targeted therapy. In some embodiments, the report is provided or transmitted to the individual, a caregiver, a healthcare provider, a physician, an oncologist, an electronic medical record system, a hospital, a clinic, a third-party payer, an insurance company, or a government office, e.g., as described in further detail above. In some embodiments, the report is transmitted via a computer network or a peer-to-peer connection. In some embodiments, an individual is administered a treatment based, at least in part, on the report. In some instances, all or a portion of the report may be displayed in a graphical user interface of an online or web-based healthcare portal.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. ARTICLES OF MANUFACTURE OR KITS

Provided herein are kits or articles of manufacture comprising one or more reagents for detecting an IGF1R polypeptide having an activating mutation, or a nucleic acid molecule encoding an IGF1R polypeptide having an activating mutation, of the disclosure, in a sample.

In some embodiments, the kits or articles of manufacture comprise one or more probes of the disclosure for detecting a nucleic acid molecule encoding an IGF1R polypeptide having an activating mutation of the disclosure in a sample, e.g., according to any detection method known in the art or described herein. In some embodiments, the kits or articles of manufacture comprise one or more baits (e.g., one or more bait molecules) of the disclosure for a nucleic acid molecule encoding an IGF1R polypeptide having an activating mutation of the disclosure in a sample, e.g., according to any detection method known in the art or described herein. In some embodiments, the kits or articles of manufacture comprise one or more oligonucleotides (e.g., one or more primers) of the disclosure for detecting a nucleic acid molecule encoding an IGF1R polypeptide having an activating mutation of the disclosure in a sample, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of an IGF1R nucleic acid molecule of the disclosure (e.g., a wild type IGF1R gene, as described herein). In some embodiments, one or more oligonucleotides, primers, probes or baits are capable of hybridizing to a nucleic acid molecule encoding an IGF1R polypeptide having an activating mutation of the disclosure, or to a wild-type counterpart of the IGF1R nucleic acid molecule (e.g., a wild type IGF1R gene, as described herein). In some embodiments, the one or more oligonucleotides, primers, probes or baits of the present disclosure are capable of distinguishing a nucleic acid molecule encoding an IGF1R polypeptide having an activating mutation of the disclosure, from a wild-type counterpart of the IGF1R nucleic acid molecule (e.g., a wild type IGF1R gene, as described herein). In some embodiments, the kit is for use according to any method of detecting mutant nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, in situ hybridization, sequence-specific priming (SSP) PCR, HPLC, and mass-spectrometric genotyping. In some embodiments, a kit provided herein further comprises instructions for detecting a nucleic acid molecule encoding an IGF1R polypeptide having an activating mutation of the disclosure, e.g., using one or more oligonucleotides, primers, probes or baits of the present disclosure.

In some embodiments, the kits or articles of manufacture comprise one or more antibodies or antibody fragments of the disclosure for detecting an IGF1R polypeptide having an activating mutation of the disclosure in a sample, e.g., according to any detection method known in the art or described herein. In some embodiments, the kit or article of manufacture comprises a reagent (e.g., one or more antibodies or antibody fragments of the present disclosure) for detecting the wild-type counterparts of an IFG1R polypeptide provided herein (e.g., a wild type IGF1R polypeptide as described herein). In some embodiments, the kits or articles of manufacture comprise one or more antibodies or antibody fragments of the present disclosure capable of binding to an IGF1R polypeptide having an activating mutation provided herein, or to wild-type counterparts of the IGF1R polypeptide provided herein (e.g., a wild type IGF1R polypeptide as described herein). In some embodiments, the kit is for use according to any protein or polypeptide detection assay known in the art or described herein, such as mass spectrometry (e.g., tandem mass spectrometry), a reporter assay (e.g., a fluorescence based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting an IGF1R polypeptide having an activating mutation of the disclosure, e.g., using one or more antibodies or antibody fragments of the present disclosure.

Further provided herein are kits or articles of manufacture comprising an anti-cancer therapy, such as an anti-cancer therapy described herein, e.g., an IGF1R-targeted therapy, and a package insert comprising instructions for using the anti-cancer therapy in a method of treating or delaying progression of cancer, e.g., by administration to an individual from whom a sample comprising an IGF1R polypeptide having an activating mutation, or a nucleic acid molecule encoding an IGF1R polypeptide having an activating mutation, of the disclosure has been obtained. In some embodiments, the anti-cancer therapy is any of the anti-cancer therapies described herein for use in any of the methods for treating or delaying progression of cancer of the disclosure.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting an IGF1R polypeptide having an activating mutation, or a nucleic acid molecule encoding an IGF1R polypeptide having an activating mutation, of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting an IGF1R polypeptide having an activating mutation, or a nucleic acid molecule encoding an IGF1R polypeptide having an activating mutation, of the disclosure, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. EXPRESSION VECTORS, HOST CELLS AND RECOMBINANT CELLS

Provided herein are vectors comprising or encoding an IGF1R polypeptide having an activating mutation of the disclosure, or a bait, a probe, or an oligonucleotide described herein, or fragments thereof.

In some embodiments, a vector provided herein comprises or encodes an IGF1R polypeptide having an activating mutation of the disclosure, or a nucleic acid molecule encoding an IGF1R polypeptide having an activating mutation described herein.

In some embodiments, a vector provided herein is a nucleic acid molecule capable of transporting another nucleic acid to which it has been linked (e.g., nucleic acid molecules encoding an IGF1R polypeptide having an activating mutation, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising mutant nucleic acid molecules, baits, probes, or oligonucleotides described herein, or fragments thereof) are also contemplated herein, including, e.g., replication defective retroviruses, adenoviruses and adeno-associated viruses.

In some embodiments, a vector provided herein comprises a nucleic acid molecule encoding an IGF1R polypeptide having an activating mutation, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., nucleic acid molecules encoding an IGF1R polypeptide having an activating mutation, baits, probes, or oligonucleotides described herein, or fragments thereof). In some embodiments, a regulatory sequence directs tissue-specific expression of a nucleotide sequence (e.g., nucleic acid molecules encoding an IGF1R polypeptide having an activating mutation, baits, probes, or oligonucleotides described herein, or fragments thereof). In some embodiments, a regulatory sequence directs inducible expression of a nucleotide sequence (e.g., nucleic acid molecules encoding an IGF1R polypeptide having an activating mutation, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes an IGF1R polypeptide having an activating mutation, a bait, a probe, or an oligonucleotide of the disclosure in the sense or the anti-sense orientation. In some embodiments, a vector (e.g., an expression vector) provided herein is introduced into host cells to thereby produce a polypeptide, e.g., an IGF1R polypeptide having an activating mutation described herein, or a fragment or mutant form thereof.

In some embodiments, the design of a vector provided herein depends on such factors as the choice of the host cell to be transformed, the level of expression desired, and the like. In some embodiments, expression vectors are designed for the expression of the nucleic acid molecule encoding an IGF1R polypeptide having an activating mutation, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes a nucleic acid molecule encoding an IGF1R polypeptide having an activating mutation described herein, wherein the nucleotide sequence of the mutant nucleic acid molecule described herein has been altered (e.g., codon optimized) so that the individual codons for each encoded amino acid are those preferentially utilized in the host cell.

Also provided herein are host cells, e.g., comprising nucleic acid molecules encoding an IGF1R polypeptide having an activating mutation, IGF1R polypeptides having an activating mutation, baits, probes, vectors, or oligonucleotides of the disclosure. In some embodiments, a host cell (e.g., a recombinant host cell or recombinant cell) comprises a vector described herein (e.g., an expression vector described herein). In some embodiments, a nucleic acid molecule encoding an IGF1R polypeptide having an activating mutation, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non-limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

Nucleic acid molecules encoding an IGF1R polypeptide having an activating mutation, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing an IGF1R polypeptide having an activating mutation of the disclosure, e.g., by culturing a host cell described herein (e.g., into which a recombinant expression vector encoding a polypeptide has been introduced) in a suitable medium such that the mutant polypeptide is produced. In another embodiment, the method further includes isolating an IGF1R polypeptide having an activating mutation from the medium or the host cell.

### VI. EXEMPLARY EMBODIMENTS

The following exemplary embodiments are representative of some aspects of the invention:

Embodiment 1. A method for identifying an individual having a cancer for treatment with an insulin-like growth factor 1 receptor (IGF1R)-targeted therapy comprising detecting an insulin-like growth factor 1 receptor (IGF1R) polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Embodiment 2. A method of selecting a treatment for an individual having a cancer, comprising:
detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
selecting the treatment, wherein the presence of said non-frameshift insertion mutation or said substitution mutation identifies the individual as one who may benefit from treatment with an IGF1R-targeted therapy.

Embodiment 3. A method of identifying one or more treatment options for an individual having a cancer comprising:
detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
generating a report comprising one or more treatment options identified for the individual based at least in part on the detection of the IGF1R polypeptide having the mutation, or the nucleic acid molecule encoding the IGF1R polypeptide having the mutation, in the sample, wherein the one or more treatment options comprises administration of an IGF1R-targeted therapy.

Embodiment 4. A method of identifying a candidate treatment for a cancer in an individual in need thereof, comprising:
performing DNA sequencing on a sample obtained from the individual to determine a sequencing mutation profile, wherein the sequencing mutation profile identifies the presence or absence of a nucleic acid molecule that encodes an IGF1R polypeptide having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R, and
selecting a treatment for the individual based, at least in part, on the sequencing mutation profile, wherein the treatment comprises an IGF1R-targeted therapy.

Embodiment 5. A method of predicting survival of an individual having a cancer treated with a treatment comprising an IGF1R-targeted therapy, the method comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R,
wherein responsive to the acquisition of said knowledge, the individual is predicted to have longer survival when treated with the treatment comprising the IGF1R-targeted therapy, as compared to an individual whose cancer does not have the IGF1R polypeptide having the mutation or the nucleic acid molecule encoding the IGF1R polypeptide having the mutation.

Embodiment 6. A method of monitoring, evaluating, or screening an individual having a cancer, comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R;
wherein responsive to the acquisition of said knowledge, the individual is predicted to have acquired resistance to a prior anti-cancer therapy administered to the individual, the individual is predicted to respond to an IGF1R-targeted therapy, and/or the individual is predicted to have poor prognosis, as compared to an individual whose cancer does not comprise the IGF1R polypeptide having the mutation or the nucleic acid molecule encoding the IGF1R polypeptide having the mutation.

Embodiment 7. The method of any one of embodiments 1-6, further comprising administering an effective amount of the IGF1R-targeted therapy to the individual, thereby treating the cancer.

Embodiment 8. A method of detecting a nucleic acid molecule encoding an IGF1R polypeptide having a mutation in a sample from an individual having a cancer, wherein the mutation is a non-frameshift insertion mutation at or following any one or more of amino acids 653-667 of IGF1R or within a tyrosine kinase domain of IGF1R, or a substitution or insertion mutation at D555 of IGF1R, comprising:
(a) providing a plurality of nucleic acid molecules obtained from a sample from the individual, wherein the plurality of nucleic acid molecules comprises a fragment of the nucleic acid molecule encoding the IGF1R polypeptide comprising the mutation;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more of the plurality of sequence reads correspond to the nucleic acid molecule encoding the IGF1R polypeptide having the mutation;
(f) analyzing the plurality of sequence reads for the presence or absence of the nucleic acid molecule encoding the IGF1R polypeptide having the mutation; and
(g) based on the analyzing step, detecting the presence or absence of the nucleic acid molecule encoding the IGF1R polypeptide having the mutation in the sample.

Embodiment 9. The method of embodiment 8, wherein the one or more adapters comprise amplification primers, flow cell adapter sequences, substrate adapter sequences, sample index sequences, or unique molecular identifier (UMI) sequences.

Embodiment 10. The method of embodiment 8 or 9, wherein the amplified nucleic acid molecules are captured by hybridization with one or more bait molecules.

Embodiment 11. A method of detecting a nucleic acid molecule encoding an IGF1R polypeptide having a mutation in a sample from an individual having a cancer, wherein the mutation is a non-frameshift insertion mutation at or following any one or more of amino acids 653-667 of IGF1R or within a tyrosine kinase domain of IGF1R, or a substitution or insertion mutation at D555 of IGF1R, the method comprising:
(a) providing the sample from the individual, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising a nucleotide sequence encoding at least a portion of an IGF1R polypeptide having the mutation, thereby making an enriched sample;
(e) sequencing, by a sequencer, the enriched sample, thereby producing a plurality of sequence reads;
(f) analyzing the plurality of sequence reads for the presence or absence of the mutation in the nucleic acid molecule encoding the IGF1R polypeptide; and
(g) detecting, based on the analyzing step, the presence or absence of the nucleic acid molecule encoding the IGF1R polypeptide having the mutation in the sample from the individual.

Embodiment 12. The method of embodiment 11, wherein the selectively enriching comprises: (a) combining one or more bait molecules with the library, thereby hybridizing the one or more bait molecules to one or more nucleic acid molecules comprising a nucleotide sequence encoding a portion of the IGF1R polypeptide, and producing nucleic acid hybrids; and (b) isolating the nucleic acid hybrids to produce the enriched sample.

Embodiment 13. The method of any one of embodiments 8-12, wherein the sequencer comprises a next-generation sequencer.

Embodiment 14. The method of any one of embodiments 8-13, wherein the amplifying comprises performing a polymerase chain reaction (PCR) amplification technique, a non-PCR amplification technique, or an isothermal amplification technique.

Embodiment 15. A method of treating a cancer, or delaying the progression of cancer, in an individual, comprising:
detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
administering an effective amount of an IGF1R-targeted therapy to the individual.

Embodiment 16. The method of embodiment 7 or 15, wherein the IGF1R-targeted therapy is administered to the individual in response to a determination of the presence of the non-frameshift insertion mutation or the substitution mutation.

Embodiment 17. A method of identifying a cancer in an individual as an adenoid cystic carcinoma comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R,
wherein the presence of the mutation in IGF1R indicates an increased likelihood that the cancer is an adenoid cystic carcinoma.

Embodiment 18. The method of embodiment 17, wherein the cancer was previously identified as a cancer other than an adenoid cystic carcinoma.

Embodiment 19. The method of embodiment 17 or 18, further comprising identifying a basaloid appearance, presence of cribriform architecture, or stroma characteristic of an adenoid cystic carcinoma, within a histological image of the cancer.

Embodiment 20. The method of any one of embodiments 17-19, further comprising administering an IGF1R-targeted therapy to the individual, thereby treating the cancer.

Embodiment 21. The method of embodiment 20, wherein the IGF1R-targeted therapy is administered to the individual in response to a determination of the presence of the non-frameshift insertion mutation or the substitution mutation.

Embodiment 22. The method of any one of embodiments 1-7 and 15-21, wherein detecting comprises selectively enriching for one or more nucleic acid molecules in the sample comprising nucleotide sequences encoding a portion of the IGF1R polypeptide to produce an enriched sample.

Embodiment 23. The method of embodiment 22, wherein the selectively enriching comprises: (a) combining one or more bait molecules with the sample, thereby hybridizing the one or more bait molecules to one or more nucleic acid molecules in the sample comprising a nucleotide sequence encoding a portion of the IGF1Rpolypeptide, thereby producing nucleic acid hybrids; and (b) isolating the nucleic acid hybrids to produce the enriched sample.

Embodiment 24. The method of embodiment 23, wherein the one or more bait molecules comprise a capture nucleic acid molecule configured to hybridize to a nucleotide sequence corresponding to IGF1R.

Embodiment 25. The method of embodiment 24, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Embodiment 26. The method of any one of embodiments 13 and 23-25, wherein the one or more bait molecules are conjugated to an affinity reagent or to a detection reagent.

Embodiment 27. The method of embodiment 26, wherein the affinity reagent is an antibody, an antibody fragment, or biotin, or wherein the detection reagent is a fluorescent marker.

Embodiment 28. The method of any one of embodiments 24-27, wherein the capture nucleic acid molecule comprises a DNA, RNA, or mixed DNA/RNA molecule.

Embodiment 29. The method of any one of embodiments 22-28, wherein the selectively enriching comprises amplifying the one or more nucleic acid molecules comprising nucleotide sequences corresponding to IGF1R using a polymerase chain reaction (PCR) to produce an enriched sample.

Embodiment 30. The method of any one of embodiments 22-29, further comprising sequencing the enriched sample.

Embodiment 31. The method of any one of embodiments 1-30, wherein the sample comprises a mixture of cancer nucleic acid molecules and non-cancer nucleic acid molecules.

Embodiment 32. The method of embodiment 31, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Embodiment 33. The method of any one of embodiments 1-32, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

Embodiment 34. The method of any one of embodiments 4, 8-14 and 30-33, wherein the sequencing comprises use of a massively parallel sequencing (MPS) technique, whole genome sequencing (WGS), whole exome sequencing, targeted sequencing, direct sequencing, or a Sanger sequencing technique; optionally wherein the sequencing comprises a massively parallel sequencing technique, and the massively parallel sequencing technique comprises next-generation sequencing (NGS).

Embodiment 35. The method of any one of embodiments 1, 2, and 4-34, further comprising generating a report, wherein the report: (a) indicates the presence or absence of the IGF1R polypeptide having the mutation or the nucleic acid molecule encoding the IGF1R polypeptide having the mutation in a sample from the individual; and/or (b) indicates a treatment or one or more treatment options identified or selected for the individual based at least in part on the detection of the IGF1R polypeptide having the mutation, or the nucleic acid molecule encoding the IGF1R polypeptide having the mutation, in the sample, wherein the one or more treatment options comprises administration of an IGF1R-targeted therapy.

Embodiment 36. The method of any one of embodiments 1-35, further comprising generating a molecular profile for the individual, based, at least in part, on detecting or acquiring knowledge of the IGF1R polypeptide having the mutation, or the nucleic acid molecule encoding the IGF1R polypeptide having the mutation, in the sample from the individual.

Embodiment 37. The method of embodiment 36, wherein the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 38. The method of embodiment 36 or 37, wherein the molecular profile for the individual comprises results from a nucleic acid sequencing-based test.

Embodiment 39. The method of any one of embodiments 36-38, further comprising selecting a treatment, administering a treatment, or applying a treatment to the individual based on the generated molecular profile, wherein the treatment comprises an IGF1R-targeted therapy.

Embodiment 40. The method of any one of embodiments 36-39, further comprising generating a report, wherein the report comprises the molecular profile for the individual.

Embodiment 41. The method of embodiment 40, wherein the report further comprises information on a treatment or one or more treatment options identified or selected for the individual based, at least in part, on the molecular profile for the individual, wherein the treatment or one or more treatment options comprise an IGF1R-targeted therapy.

Embodiment 42. The method of any one of embodiments 3 and 35-41, further comprising providing the report to the individual, a caregiver, a healthcare provider, a physician, an oncologist, an electronic medical record system, a hospital, a clinic, a third-party payer, an insurance company, or a government office.

Embodiment 43. The method of any one of embodiments 1-42, wherein the individual is a human.

Embodiment 44. The method of any one of embodiments 1-43, further comprising obtaining the sample from the individual.

Embodiment 45. The method of any one of embodiments 1-44, wherein the sample is obtained or derived from the cancer.

Embodiment 46. The method of any one of embodiments 1-45, wherein the sample comprises a tissue biopsy sample, a liquid biopsy sample, or a normal control.

Embodiment 47. The method of any one of embodiments 1-46, wherein the sample is from a tumor biopsy, tumor specimen, or circulating tumor cell.

Embodiment 48. The method of any one of embodiments 1-47, wherein the sample is a liquid biopsy sample comprising blood, serum, plasma, cerebrospinal fluid, sputum, stool, urine, or saliva.

Embodiment 49. The method of any one of embodiments 1-48, wherein the sample comprises cells and/or nucleic acids from the cancer.

Embodiment 50. The method of embodiment 48, wherein the sample comprises mRNA, DNA, circulating tumor DNA (ctDNA), cell-free DNA, or cell-free RNA from the cancer.

Embodiment 51. The method of any one of embodiments 1-50, wherein the sample is a liquid biopsy sample comprising circulating tumor cells (CTCs).

Embodiment 52. The method of any one of embodiments 1-51, wherein the sample is a liquid biopsy sample comprising cell-free DNA (cfDNA), circulating tumor DNA (ctDNA), or any combination thereof.

Embodiment 53. The method of any one of embodiments 1-3, 5-7, 15-21, and 35-47, wherein the IGF1R polypeptide having the mutation is detected in the sample by one or more of: immunoblotting, enzyme linked immunosorbent assay (ELISA), immunohistochemistry, or mass spectrometry.

Embodiment 54. A method of treating an individual having cancer comprising:
selecting the individual for treatment based on the cancer having an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
administering an effective amount of an IGF1R-targeted therapy to the selected individual.

Embodiment 55. The method of any one of embodiments 1-54, wherein the cancer is a breast cancer, a head and neck cancer, an adenoid cystic carcinoma, an anal cancer, a bladder cancer, a uterine cancer, a lung cancer, a skin cancer, a neuroendocrine cancer, an ovarian cancer, or a prostate cancer.

Embodiment 56. The method of any one of embodiments 1-55, wherein the cancer is an adenoid cystic carcinoma.

Embodiment 57. The method of any one of embodiments 1-56, wherein the cancer is a basaloid cancer.

Embodiment 58. The method of any one of embodiments 1-57, wherein the cancer is a head or neck cancer.

Embodiment 59. The method of any one of embodiments 1-58, wherein the cancer is a salivary gland cancer.

Embodiment 60. The method of any one of embodiments 1-59, wherein the cancer is a basaloid salivary gland cancer.

Embodiment 61. The method of any one of embodiments 1-60, wherein the cancer is a metastatic cancer.

Embodiment 62. The method of any one of embodiments 1-60, wherein the cancer is a primary cancer.

Embodiment 63. The method of any one of embodiments 1-62, wherein the mutation is a non-frameshift insertion mutation at or following any one or more of amino acids 653-667 of IGF1R.

Embodiment 64. The method of any one of embodiments 1-63, wherein the mutation is a non-frameshift insertion mutation at or following any one or more of amino acids 663-666 of IGF1R.

Embodiment 65. The method of any one of embodiments 1-64, wherein the non-frameshift insertion mutation comprises a duplication of Y662 and C663.

Embodiment 66. The method of any one of embodiments 1-65, wherein the non-frameshift insertion mutation comprises a duplication.

Embodiment 67. The method of any one of embodiments 1-65, wherein the non-frameshift insertion mutation within the comprises a delins mutation.

Embodiment 68. The method of any one of embodiments 1-67, wherein the mutation is a non-frameshift insertion mutation at or following any one or more of amino acids 653-667 of IGF1R, and wherein the non-frameshift insertion mutation is 3 to 13 amino acids in length.

Embodiment 69. The method of any one of embodiments 1-68, wherein the mutation comprises a Q653_K665dup mutation, a Q653_S664dup mutation, a R659_C663dup mutation, a Y656_C663dup mutation, a L657_S664dup mutation, a N661_D666dup mutation, a Y662_S664dup mutation, a C663_S664insYRHNYC mutation, a C663_S664insRHNYC mutation, a C663_S664insYGYLYRHNYC mutation, a C663_S664insYRHNYC mutation, a C663_S664insYLYRHNYC mutation, S664_K665insYCS mutation, a S664_K665insRHNYCS mutation, a S664_K665insLYRHNYCS mutation, a K665_D666insGYCSK mutation, K665_D666insQDGYLYRHNYCSK mutation, or a D666_K667insDNYCSK mutation.

Embodiment 70. The method of any one of embodiments 1-62, wherein the mutation is the non-frameshift insertion mutation within the tyrosine kinase domain of IGF1R.

Embodiment 71. The method of any one of embodiments 1-62 and 70, wherein the non-frameshift insertion mutation within the tyrosine kinase domain of IGF1R is at or follows any one of amino acids 1034-1049 of IGF1R.

Embodiment 72. The method of any one of embodiments 1-62, 70, and 71, wherein the non-frameshift insertion mutation within the tyrosine kinase domain of IGF1R comprises a delins mutation.

Embodiment 73. The method of any one of embodiments 1-62 and 70-72, wherein the non-frameshift insertion mutation within the tyrosine kinase domain of IGF1R is 1 to 9 amino acids in length.

Embodiment 74. The method of any one of embodiments 1-62 and 70-73, wherein the mutation comprises a T1034_V1035insN mutation, a A1039dup mutation, a A1039_S1040insA mutation, a M1041_R1042insL mutation, a R1042_E1046dup, a E1043_R1044insK mutation, a R1044_I1045insM mutation, a R1044_I1045insR mutation, a I1045_E1046insD mutation, a E1046_F1047insRERIE mutation, a E1046_F1047insAAMRERIE mutation, a F1047delinsLL mutation, a L1048dup mutation, a L1048_N1049insL mutation, or a N1049delinsLD mutation.

Embodiment 75. The method of any one of embodiments 1-62, wherein the mutation is the substitution or insertion mutation at D555 of IGF1R.

Embodiment 76. The method of any one of embodiments 1-62 and 75, wherein the mutation is the substitution mutation at D555 of IGF1R.

Embodiment 77. The method of any one of embodiments 1-62, 75 and 76, wherein the mutation is a D555A mutation, a D555Y mutation, a D555E mutation, a D555N mutation, or a D555G mutation.

Embodiment 78. The method of any one of embodiments 1-62, 75, and 76, wherein the mutation is D555_L556insVD.

Embodiment 79. The method of any one of embodiments 1-78, wherein the cancer is negative for a MYB, MYBL1, or NOTCH1 driver mutation.

Embodiment 80. The method of any one of embodiments 1-79, wherein the cancer is negative for a MYB-NFIB fusion.

Embodiment 81. The method of any one of embodiments 1-80, wherein the IGF1R having the mutation comprises the tyrosine kinase domain, or a fragment of the tyrosine kinase domain, having a kinase activity.

Embodiment 82. The method of embodiment 81, wherein the kinase activity is constitutive.

Embodiment 83. The method of any one of embodiments 1-82, wherein the IGF1R having the mutation is oncogenic.

Embodiment 84. The method of any one of embodiments 1-83, wherein the IGF1R having the mutation promotes cancer cell survival, angiogenesis, cancer cell proliferation, and any combination thereof.

Embodiment 85. The method of any one of embodiments 1-7, 15, 16, and 20-84, wherein the IGF1R-targeted therapy comprises one or more of a small molecule inhibitor, an antibody, an antibody fragment, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an IGF1R mutation, an IGF1R-targeted therapy being tested in a clinical trial, a treatment for cancer comprising an IGF1R mutation being tested in a clinical trial, or any combination thereof.

Embodiment 86. The method of any one of embodiments 1-7, 15, 16, and 20-85, wherein the IGF1R-targeted therapy specifically targets IGF1R.

Embodiment 87. The method of any one of embodiments 1-7, 15, 16, and 20-86, wherein the IGF1R-targeted therapy specifically targets an IGF1R ligand.

Embodiment 88. The method of any one of embodiments 85-87, wherein the IGF1R-targeted therapy comprises an antibody or fragment thereof.

Embodiment 89. The method of any one of clams 85-88, wherein the IGF1R-targeted therapy comprises a monoclonal antibody.

Embodiment 90. The method of any one of embodiments 85-89, wherein the IGF1R-targeted therapy comprises cixutumumab, figitumumab, dalotuzumab, ganitumab, robatumumab, BMS-754807, NVP-ADW742, NVP-AEW541, OSI-906, teprotumumab, ceritinib, xentuzumab, AXL1717, IGF-MTX, W0101, FPI-1434, SCH717454, AVE1642, BIIB022, or MEDI-573.

Embodiment 91. The method of any one of embodiments 1-7, 15, 16, and 20-85, wherein the IGF1R-targeted therapy comprises a kinase inhibitor.

Embodiment 92. The method of embodiment 91, wherein the IGF1R-targeted therapy comprises a tyrosine kinase inhibitor.

Embodiment 93. The method of embodiment 91 or 92, wherein the IGF1R-targeted therapy comprises kinase inhibitor that inhibits kinase activity of the IGF1R polypeptide.

Embodiment 94. The method of any one of embodiments 91-93, wherein the IGF1R-targeted therapy comprises a multi-kinase inhibitor.

Embodiment 95. The method of any one of embodiments 91-94, wherein the IGF1R-targeted therapy comprises an IGF1R-kinase specific inhibitor.

Embodiment 96. The method of any one of embodiments 1-7, 15, 16, and 20-85, wherein the IGF1R-targeted therapy comprises a small-molecule tyrosine kinase inhibitor.

Embodiment 97. The method of embodiment 96, wherein the small-molecule tyrosine kinase inhibitor is linsitinib, ceritinib, BMS-754807, BVP 51004, XL228, or INSM-18.

Embodiment 98. The method any one of embodiments 1-7, 15, 16, and 20-85, wherein the IGF1R-targeted therapy comprises a nucleic acid that inhibits the expression of IGF1R.

Embodiment 99. The method of embodiment 98, wherein the nucleic acid is a double-stranded RNA (dsRNA), a small interfering RNA (siRNA), or a small hairpin RNA (shRNA).

Embodiment 100. The method any one of embodiments 1-7, 15, 16, and 20-85, wherein the IGF1R-targeted therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 101. The method of any one of embodiments 1-100, wherein the individual has received a prior anti-cancer treatment, or is being treated with an anti-cancer treatment; optionally wherein the cancer is resistant or refractory to the anti-cancer treatment.

Embodiment 102. The method of any one of embodiments 1-7, 15, 16, and 20-85, wherein the treatment or the one or more treatment options further comprise an additional anti-cancer therapy.

Embodiment 103. The method of embodiment 102, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Embodiment 104. The method of embodiment 103, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 105. The method of embodiment 104, wherein the nucleic acid comprises a double-stranded RNA (dsRNA), a small interfering RNA (siRNA), or a small hairpin RNA (shRNA).

Embodiment 106. A kit comprising one or more probes, baits, or oligonucleotides for detecting an IGF1R polypeptide, or a fragment thereof, having a mutation or nucleic acid molecule encoding the IGF1R polypeptide, or fragment thereof, having the mutation in a sample, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Embodiment 107. An isolated nucleic acid encoding an IGF1R polypeptide, or a fragment thereof, having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Embodiment 108. A vector comprising the nucleic acid of embodiment 107.

Embodiment 109. A host cell comprising the vector of embodiment 108.

Embodiment 110. An antibody or antibody fragment that specifically binds to encoding an IGF1R polypeptide, or a fragment thereof, having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R.

Embodiment 111. An IGF1R-targeted therapy for use in a method of treating or delaying progression of cancer, wherein the method comprises administering the IGF1R-targeted therapy to an individual having cancer, wherein an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R, is detected in a sample obtained from the individual.

Embodiment 112. An IGF1R-targeted therapy for use in the manufacture of a medicament for treating or delaying progression of cancer, wherein the medicament is to be administered to an individual having cancer, wherein an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R, is detected in a sample obtained from the individual.

Embodiment 113. A system, comprising:
a memory configured to store one or more programs, and
one or more processors configured to execute the one or more programs, the one or more programs when executed by the one or more processors are configured to:
(a) obtain a plurality of sequence reads of one or more nucleic acid molecules, wherein the one or more nucleic acid molecules are derived from a sample obtained from an individual having a cancer;
(b) analyze the plurality of sequence reads for the presence of a nucleic acid molecule encoding an IGF1R polypeptide having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
(c) detect, based on the analyzing, the nucleic acid molecule encoding the IGF1R polypeptide having the mutation in the sample.

Embodiment 114. A non-transitory computer readable storage medium comprising one or more programs executable by one or more computer processors for performing a method, the method comprising:
(a) obtain a plurality of sequence reads of one or more nucleic acid molecules, wherein the one or more nucleic acid molecules are derived from a sample obtained from an individual having a cancer;
(b) analyze the plurality of sequence reads for the presence of a nucleic acid molecule encoding an IGF1R polypeptide having a mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
(c) detect, based on the analyzing, the nucleic acid molecule encoding the IGF1R polypeptide having the mutation in the sample.

Embodiment 115. The system of embodiment 113, or the non-transitory computer readable storage medium of embodiment 114, wherein the plurality of sequence reads is obtained by sequencing.

Embodiment 116. The system or non-transitory computer readable storage medium of embodiment 115, wherein the sequencing comprises use of a massively parallel sequencing (MPS) technique, whole genome sequencing (WGS), whole exome sequencing, targeted sequencing, direct sequencing, or a Sanger sequencing technique.

Embodiment 117. The system or non-transitory computer readable storage medium of embodiment 116, wherein the sequencing comprises use of a massively parallel sequencing (MPS) technique, and wherein the massively parallel sequencing technique comprises next generation sequencing (NGS).

Embodiment 118. The system or non-transitory computer readable storage medium of any one of embodiments 113-117, wherein the one or more programs are further configured to generate, based at least in part on the detecting, a molecular profile for the sample.

Embodiment 119. The system or non-transitory computer readable storage medium of embodiment 118, wherein the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 120. The system or non-transitory computer readable storage medium of embodiment 118 or 119, wherein the molecular profile further comprises results from a nucleic acid sequencing-based test.

### EXAMPLES

The invention will be more fully understood by reference to the following examples. They should not, however, be construed as limiting the scope of the invention. It is understood that the examples and embodiments described herein are for illustrative purposes only, and that various modifications or changes in light thereof will be suggested to persons skilled in the art and are to be included within the spirit and purview of this application and scope of the appended claims.

### Example 1—Pan-Cancer Landscape of IGF1R Mutations Highlights Hotspot Non-Frameshift Insertions, which are Enriched in Adenoid Cystic Carcinomas

A retrospective cohort study was performed as outlined in the study design flowchart provided in FIG. 1. Solid tumor specimens were submitted for comprehensive genomic profiling (CGP) in the course of clinical care. All patients with IGF1R single nucleotide variants, delins, rearrangements, or copy number alterations were included in downstream analyses. All patients without IGF1R variants from the same period were used for comparisons.

To detect IGF1R genomic alterations, CGP was performed on formalin-fixed, paraffin embedded tissue, wherein sections were macrodissected to achieve >20% estimated percent tumor nuclei (% TN) in each case, where % TN is calculated as the number of tumor cells divided by total number of nucleated cells. Then DNA was extracted, quantified, and enriched via adaptor ligation hybrid capture for all coding exons of 236, 315, or 405 cancer-related genes plus select introns from 19, 28, or 31 genes that are frequently rearranged in cancer. Sequencing of captured libraries was performed using an Illumina sequencing system to a mean exon coverage depth of targeted regions of >500×. Processed and aligned reads were analyzed for single nucleotide variants, delins, rearrangements, and copy number alterations. Tumor purity was estimated based on genome-wide aneuploidy and allelic imbalance. Tumor mutational burden (TMB, mutations/Mb) was determined on 0.8-1.1 Mb of sequenced DNA. TMB high (>10 mut/Mb) samples were excluded from further analysis to avoid over-interpretation of passenger mutations. Additionally, duplicate samples, subjects that did not consent to research, samples with significant contamination (including from transplants), and samples not run on one of two bait sets were excluded from further analysis. In addition, IGF1R variants with unknown or predicted benign functional status were filtered from the cohort.

Differences among categorical variables were assessed using Fisher's exact test. Statistical analyses were performed using R version 3.6.2 (R Foundation for Statistical Computing). Statistical tests were 2-sided and used a significance threshold of p<0.05. Reported p-values were adjusted for multiple testing using the False Discovery Rate (FDR) method.

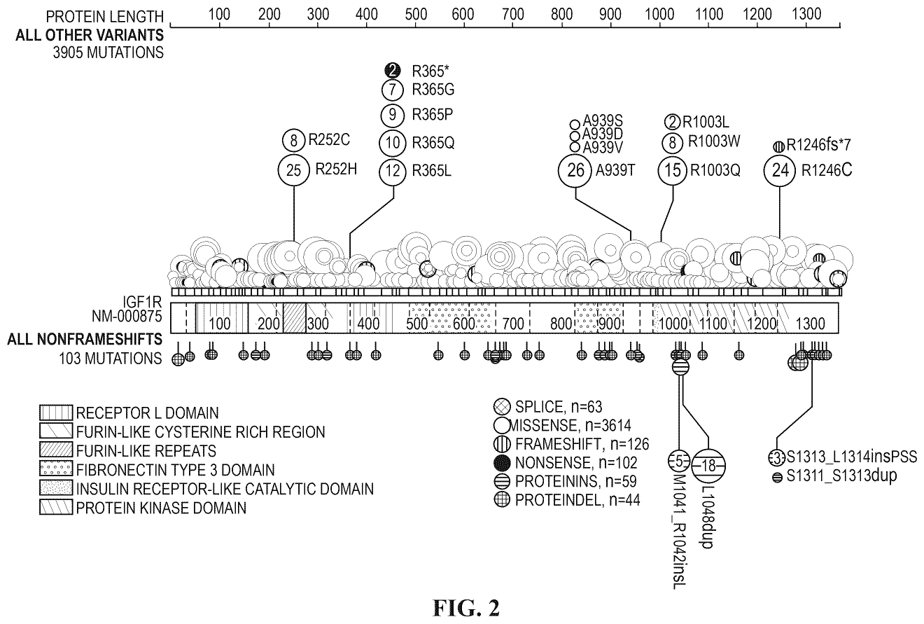

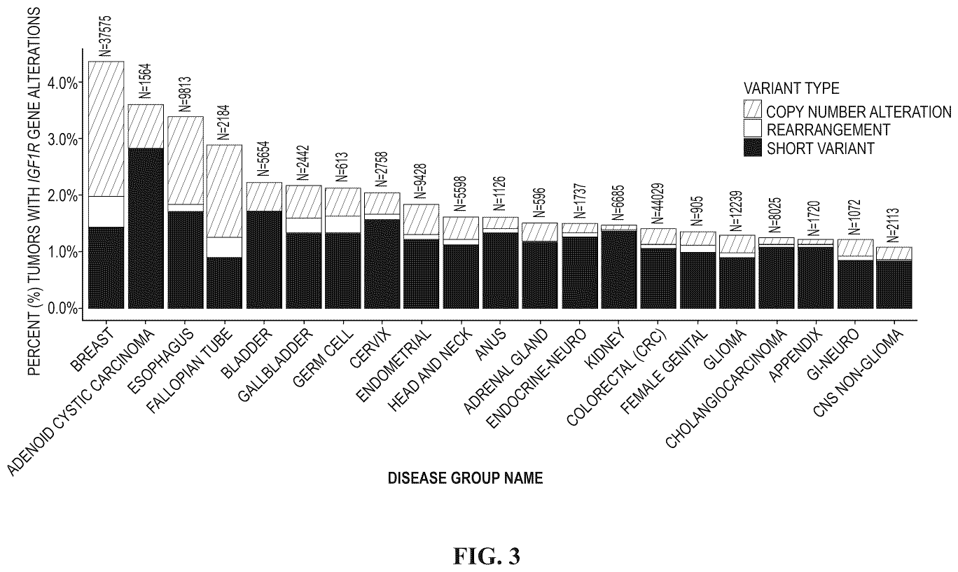

The data from the described sequencing and analysis showed overall, 1.99% (6,502/326,911) of samples exhibited at least one IGF1R mutation (FIG. 2). IGF1R alterations were seen in many cancer types and were most frequent in breast (3.96%, 1,489/37,575), adenoid cystic carcinoma (3.52%, 55/1,564), and esophagus (3.30%, 324/9,813) cancers (FIG. 3). The most common IGF1R mutations included substitution mutations (52.38%, 3,614/6,899), amplifications (34.29%, 2,366/6,899), and rearrangements (6.32%, 436/6,899).

Review of the spatial distribution of pan-cancer IGF1R mutations revealed that single nucleotide alterations were dispersed throughout the gene. In contrast, rare non-frameshift insertions were localized to specific hotspots in the gene, involving either the kinase domain or the structurally critical fibronectin 3 domain coded in exon 9. (FIG. 2, lower track). Table 1 provides a summary of all non-frameshift IGF1R mutations identified across pan-cancer samples, including those localized to specific hotspots in the gene. Similar alterations are a well-described mechanism of activation for other kinases, but this phenomenon has not been previously described in IGF1R.

TABLE 1
Summary of all non-frameshift, non-substitution IGFIR
alterations found in 6,502 samples.

| Coding Sequence | | | ACC |
|---|---|---|---|
| Variant | Protein Sequence Variant | Count | Count |
| C.3141_3143dup | P.Leu1048dup | 18 | 4 |
| C.3122_3124dup | P.Met1041_arg1042insLeu | 5 | 3 |
| C.3114_3116dup | P.Ala1039dup | 1 | 1 |
| C.3131_3132insaat | P.Arg1044_Ile1045insMet | 4 | 1 |
| C.1957_1995dup | P.Gln653_Lys665dup | 1 | 1 |
| C.3102_3103insAAT | P.Thr1034_Val1035insAsn | 1 | 1 |
| C.1983_1991dup | P.Tyr662_Ser664dup | 1 | 1 |
| C.3114_3116del | P.Ala1039del | 1 | 0 |
| C.3124_3138dup | P.Arg1042_Glu1046dup | 2 | 0 |
| C.1975_1989dup | P.Arg659_Cys663dup | 1 | 0 |
| C.3145delinsCTCG | P.Asn1049delinsLeuAsp | 1 | 0 |
| C.1979_1996dup | P.Asn661_Asp666dup | 1 | 0 |
| C.1961_1990dup | P.Cys663_Ser664insTyrGlyTyrLeu | 1 | 0 |
| | TyrArgHisAsnTyrCys | | |
| C.1956_1991dup | P.Gln653_Ser664dup | 1 | 0 |
| C.3130_3131insAAA | P.Glu1043_Arg1044insLys | 2 | 0 |
| C.3135_3137dup | P.Ile 1045_Glu1046insAsp | 1 | 0 |
| C.1969_1992dup | P.Leu657_Ser664dup | 1 | 0 |
| C.3141delinsGTTG | P.Phe 1047delinsLeuLeu | 1 | 0 |
| C.1967_1990dup | P.Tyr656_Cys663dup | 1 | 0 |
| C.105_107del | P.Gly36del | 1 | 1 |
| C.2707_2724del | P.Ala903_Thr908del | 1 | 0 |
| C.2827_2874dup | P.Ala943_Phe958dup | 1 | 0 |
| C.665_667delinsAGC | P.Arg222_Ala223delinsGlnPro | 2 | 0 |
| C.1095_1097del | P.Arg366del | 1 | 0 |
| C.1948_1959del | P.Arg650_Gln653del | 1 | 0 |
| C.2257_2295del | P.Arg753_Ile765del | 1 | 0 |
| C.2684_2692del | P.Arg895_Asn897del | 1 | 0 |
| C.2709_2711del | P.Arg904del | 1 | 0 |
| C.3979_3984del | P.Asn1327_Gly1328del | 1 | 0 |
| C.3882_3887dup | P.Asp1294_Leu1295dup | 1 | 0 |
| C.2022_2072del | P.Asp675_Val691del | 1 | 0 |
| C.2626_2627insGTCTC | P.asp875_Gln876insArgLeuLeuAsp | 1 | 0 |
| TTGGATCTGGAAC | LeuGlu | | |
| C.4024_4044del | P.Glu1342_His1348del | 1 | 0 |
| C.562_564del | P.Glu188del | 1 | 0 |
| C.2045_2047del | P.Glu682del | 1 | 0 |
| C.2169_2171del | P.Glu723_Asn724delinsAsp | 1 | 0 |
| C.2631_2657dup | P.Glu878_Arg886dup | 1 | 0 |
| C.902_904del | P.Gly301del | 1 | 0 |
| C.2846_2848delinsAAA | P.Gly949_Gly950delinsGluArg | 1 | 0 |
| C.2845_2877dup | P.Gly949_His959dup | 1 | 0 |
| C.2848_2877dup | P.Gly950_His959dup | 1 | 0 |
| C.1141_1143del | P.Ile381del | 1 | 0 |
| C.115_126del | P.Ile39_Asp42del | 1 | 0 |
| C.2004_2072del | P.Ile668_ Val691delinsmet | 1 | 0 |
| C.2009_2062del | P.Ile670_Pro687del | 1 | 0 |
| C.2402_2416del | P.Ile801_Cys806delinsSer | 1 | 0 |
| C.2584_2588delinsCA | P.Ile862_Leu863delinsGln | 1 | 0 |
| C.3945_3950dup | P.Leu1316_Pro1317dup | 1 | 0 |
| C.445_468del | P.Leu149_Asp156del | 1 | 0 |
| C.48_50del | P.Leu17del | 2 | 0 |
| C.856_858del | P.Leu286del | 1 | 0 |
| C.1804_1818del | P.Leu602_Thr606del | 1 | 0 |
| C.259_261del | P.Leu87del | 1 | 0 |
| C.2818_2829del | P.Leu940_Ala943del | 1 | 0 |
| C.3163_3165del | P.Lys1055del | 1 | 0 |
| C.3262_3273del | P.Lys1088_Leu1091del | 1 | 0 |
| C.518_520dup | P.Lys173_Pro174insGgln | 1 | 0 |
| C.3810_3811delinsTC | P.Met1270_Glu1271delinsIleGln | 1 | 0 |
| C.1653_1654delinsCT | P.Met551_Val552delinsIleLeu | 1 | 0 |
| C.3836_3838del | P.Phe1279del | 3 | 0 |
| C.1259_1261del | P.Phe420del | 1 | 0 |
| C.3862_3879del | P.Pro1288_Leu1293del | 1 | 0 |
| C.3869_3877del | P.Pro1290_Glu1292del | 3 | 0 |
| C.3944_3958dup | P.Pro1315_Arg1319dup | 1 | 0 |
| C.568_582del | P.Pro190_Lys194del | 1 | 0 |
| C.2060_2155del | P.Pro687_Tyr718del | 1 | 0 |
| C.3931_3939dup | P.Ser1311_Ser1313dup | 1 | 0 |
| C.3932_3940dup | P.Ser1313_Leu1314insProSerSer | 3 | 0 |
| C.3961_3969del | P.Ser1321_His1323del | 1 | 0 |
| C.1637_1645del | P.Ser546_Ser548del | 1 | 0 |
| C.2186_2191del | P.Ser729_Ile730del | 1 | 0 |
| C.3572_3575delinsTTTT | P.Thr1191_Tyr1192delinsIlePhe | 1 | 0 |
| C.2517_2519del | P.Trp840del | 1 | 0 |
| C.3493_3522del | P.Tyr1165_Leu1174del | 1 | 0 |
| C.959_961dup | P.Tyr320dup | 1 | 0 |
| C.2018_2026del | P.Tyr673_Gly676delinsCys | 1 | 0 |
| C.4002_4007del | P.Val1335_Leu1336del | 1 | 0 |
| C.237_245del | P.Val80_Thr82del | 1 | 0 |
| C.2825_2827del | P.Val942del | 1 | 0 |
| C.2857_2858insATTAT | P.Val952_Ile953insAsnTyrAlaVal | 1 | 0 |
| GCTGTCCTGTTGATC | LeuLeuIleValGlyGlyLeuVal | | |
| GTGGGAGGGTTGGTGA | | | |
| C.2850_2873dup | P.Val957_Phe958insLeuLeuValIle | 1 | 0 |
| | MetLeuTyrVal | | |

ACC = Adenoid cystic carcinoma.

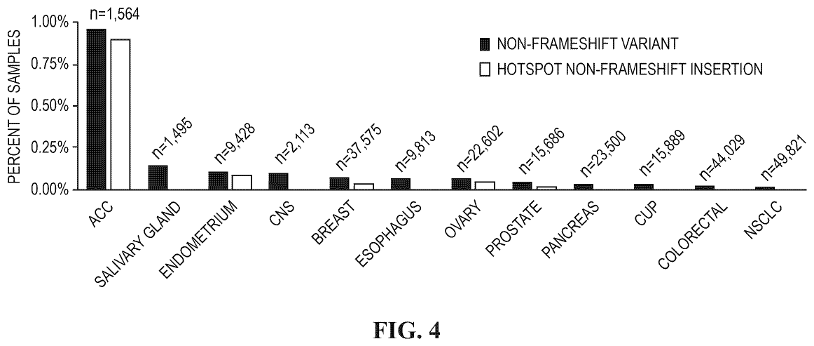

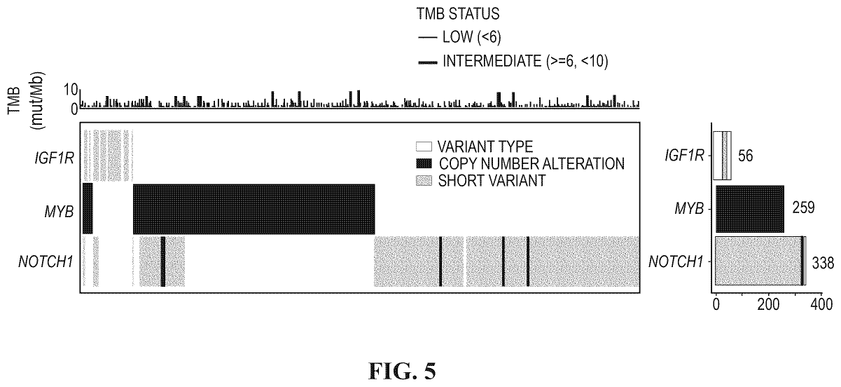

IGF1R non-frameshift insertions were markedly enriched in ACC compared to other cancer types. Non-frameshift insertions were found in 0.90% (14/1564) of ACCs, a frequency that was 12.1-fold higher than any other tumor type with at least 1,000 samples, and 27.3-fold higher than in the remainder of the pan-cancer dataset (P=2.3×10⁻¹⁷) (FIG. 4). Characteristics of ACCs and of non-ACCs with hotspot non-frameshift insertions are found below in Table 2 and Table 3, respectively. The majority (9/15, 60.0%) of ACCs with IGF1R hotspot alterations did not co-occur with the more common ACC driver alterations in MYB and NOTCH1 (FIG. 5). Samples with non-frameshift hotspot insertions had co-occurring alterations involving MYB fusions (20.0%, 3/15), NOTCH1 (26.7%, 4/15), and HRAS (13.3%, 2/15). IGF1R hotspot insertions occurring at the fibronectin 3 domain were always found to be independent of other drivers (100%, 3/3), whereas kinase domain alterations were found both independently (50%, 6/12) as well as co-occurring with other drivers (50%, 6/12).

TABLE 2
Clinical, pathologic, and genomic characteristics of ACCs with IGF1R
hotspot non-frameshift insertions. VAF = Variant allele fraction.

| | | | IGF1R Non- | | Tumor |
|---|---|---|---|---|---|
| Tissue of Origin | Sex | Age | Frameshift Insertion | VAF | Purity |
| salivary gland | F | 60 | R659_C663dup | 36.0% | 40.0% |
| salivary gland | F | 70 | Y662_S664dup | 33.5% | 58.3% |
| head and neck | M | 44 | Q653_K665dup | 17.5% | 28.3% |
| salivary gland | F | 66 | T1034_V1035insN | 39.0% | 68.5% |
| salivary gland | F | 66 | T1034_V1035insN | 39.0% | 68.5% |
| head and neck | M | 50 | A1039dup | 45.0% | 50.0% |
| head and neck | M | 51 | M1041_R1042insL | 12.3% | 31.5% |
| lung | F | 26 | M1041_R1042insL | 38.8% | 70.1% |
| breast | F | 68 | M1041_R1042insL | 25.6% | 31.8% |
| head and neck | F | 59 | R1044_11045insM | 7.1% | 30.1% |
| salivary gland | F | 61 | L1048dup | 48.0% | 69.5% |
| head and neck | M | 62 | L1048dup | 31.8% | 61.1% |
| salivary gland | M | 63 | L1048dup | 19.3% | 18.1% |
| salivary gland | F | 51 | L1048dup | 31.3% | 59.7% |
| head and neck | F | 61 | L1048dup | 34.9% | 50.7% |

TABLE 3
Clinical, pathologic, and genomic characteristics of non-ACC specimens with
IGF1R hotspot non-frameshift insertions. VAF = Variant allele fraction.

| | | | IGF1R Non-frameshift | | Tumor |
|---|---|---|---|---|---|
| Disease Ontology | Sex | Age | Insertion | VAF | Purity |
| Nasopharynx and | M | 28 | Y656_C663dup | 28.6% | 69.6% |
| paranasal sinuses | | | | | |
| undifferentiated | | | | | |
| carcinoma | | | | | |
| Bladder urothelial | M | 61 | C663_S664insYGYLYRHNYC | 24.0% | 28.5% |
| carcinoma | | | | | |
| Ovary endometrioid | F | 38 | L657_S664dup | 33.0% | 40.5% |
| adenocarcinoma | | | | | |
| Breast carcinoma (nos) | F | 60 | Q653_S664dup | 29.5% | 58.0% |
| Ovary endometrioid | F | 28 | K665_D666insGYCSK | 50.0% | 44.7% |
| adenocarcinoma | | | | | |
| Breast carcinoma (nos) | F | 67 | N661_D666dup | 36.0% | 26.0% |
| Anus squamous cell | F | 58 | M1041_R1042insL | 27.3% | 38.4% |
| carcinoma (scc) | | | | | |
| Ovary sex cord stromal | F | 57 | M1041_R1042insL | 26.8% | 42.6% |
| tumor (nos) | | | | | |
| Ovary granulosa cell | F | 77 | E1043_R1044insK | 44.1% | 85.0% |
| tumor | | | | | |
| Uterus endometrial | F | 68 | E1043_R1044insK | 7.2% | 35.0% |
| adenocarcinoma | | | | | |
| endometrioid | | | | | |
| Breast carcinoma (nos) | F | 59 | R1044_I1045insM | 38.2% | 31.1% |
| Bladder neuroendocrine | F | 34 | R1044_I1045insM | 56.2% | 40.8% |
| carcinoma | | | | | |
| Ovary granulosa cell | F | 64 | R1044_I1045insM | 49.9% | 72.8% |
| tumor | | | | | |
| Uterus endometrial | F | 62 | I1045_E1046insD | 25.1% | 30.6% |
| adenocarcinoma (nos) | | | | | |
| Uterus endometrial | F | 33 | R1042_E1046dup | 21.9% | 28.8% |
| adenocarcinoma (nos) | | | | | |
| Prostate ductal | M | 67 | R1042_E1046dup | 21.2% | 44.7% |
| adenocarcinoma | | | | | |
| Breast carcinoma (nos) | | 82 | F1047delinsLL | 45.8% | 36.7% |
| Breast invasive lobular | F | 76 | L1048dup | 17.8% | 28.5% |
| carcinoma (ilc) | | | | | |
| Prostate acinar | M | 73 | L1048dup | 17.5% | 40.6% |
| adenocarcinoma | | | | | |
| Uterus endometrial | F | 41 | L1048dup | 31.7% | 20.1% |
| adenocarcinoma | | | | | |
| endometrioid | | | | | |
| Breast invasive ductal | F | 58 | L1048dup | 20.3% | 26.9% |
| carcinoma (idc) | | | | | |
| Ovary endometrioid | F | 68 | L1048dup | 35.8% | 77.4% |
| adenocarcinoma | | | | | |
| Ovary granulosa cell | F | 78 | L1048dup | 48.9% | 53.7% |
| tumor | | | | | |
| Uterus endometrial | F | 73 | L1048dup | 28.2% | 35.0% |
| adenocarcinoma | | | | | |
| endometrioid | | | | | |
| Breast carcinoma (nos) | F | 65 | L1048dup | 31.4% | 61.0% |
| Ovary granulosa cell | F | 79 | L1048dup | 21.0% | 66.0% |
| tumor | | | | | |
| Uterus endometrial | F | 40 | L1048dup | 28.0% | 41.5% |
| adenocarcinoma | | | | | |
| endometrioid | | | | | |
| Uterus carcinosarcoma | F | 40 | L1048dup | 33.4% | 45.0% |
| Breast carcinoma (nos) | F | 57 | L1048dup | 45.6% | 44.9% |
| Breast carcinoma (nos) | F | 42 | L1048dup | 16.3% | 33.4% |
| Breast carcinoma (nos) | F | 56 | N1049delinsLD | 31.9% | 54.7% |

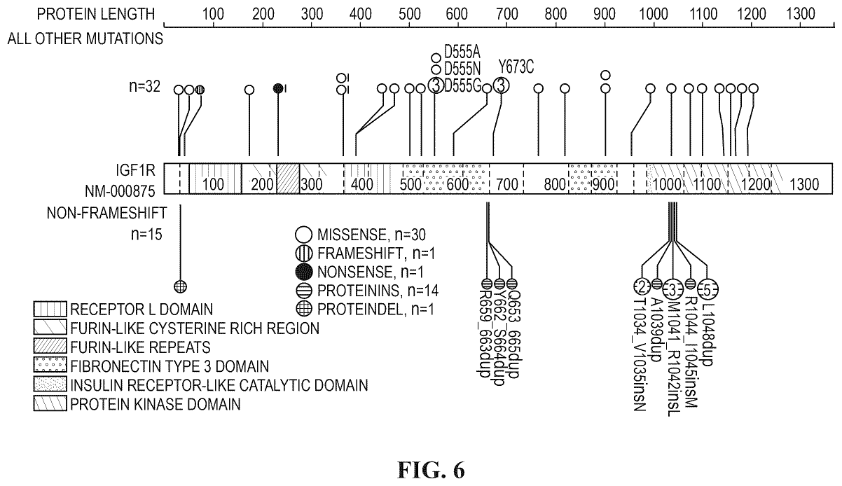

As in the pan-cancer samples, ACC non-frameshift IGF1R insertions were clustered in two distinct hotspots around amino acid (AA) positions 653-667 and 1034-1049 (FIG. 6), corresponding to the fibronectin type 3 domain and the tyrosine kinase domain, respectively. Additionally, recurrent substitution alterations occurred at a hotspot at position D555 within the fibronectin type 3 domain (e.g. FIG. 6, Table 4), wherein hotspot substitution single nucleotide variants included a D555 amino acid point mutation to: Y, N, G, A, or E. These hotspot substitution alterations provide additional support to confirm the importance of the Fibronectin 3 domain in IGF1R signaling and indicate substitution alterations at Do-F as a novel biomarker in cancer samples. Table 4 further provides additional non-frameshift mutations identified within the hotspot regions.

TABLE 4
Clinical, pathologic, and genomic characteristics of
pan-cancer specimens with IGF1R hotspot mutations.

| | | | Coding | Protein | |
|---|---|---|---|---|---|
| | | | Sequence | Sequence | Variant |
| Disease | Sex | Age | Variant | Variant | subtype |
| Lung non- | F | 71 | 1662_1663GG> | D555Y | Substitution |
| small cell lung | | | TT | | |
| carcinoma | | | | | |
| (nsclc) | | | | | |
| Melanoma | F | 60 | 1662_1663GG> | D555N | Substitution |
| | | | AA | | |
| Adenoid cystic | F | 50 | 1663G>A | D555N | Substitution |
| carcinoma | | | | | |
| Lung non- | M | 75 | 1663G>T | D555Y | Substitution |
| small cell lung | | | | | |
| carcinoma | | | | | |
| (nsclc) | | | | | |
| Lung non- | M | 83 | 1663G>T | D555Y | Substitution |
| small cell lung | | | | | |
| carcinoma | | | | | |
| (nsclc) | | | | | |
| Head and neck | M | 50 | 1663G>A | D555N | Substitution |
| Prostate | M | 85 | 1663G>T | D555Y | Substitution |
| Skin | M | 66 | 1663G>A | D555N | Substitution |
| Unknown | F | 65 | 1663G>T | D555Y | Substitution |
| primary | | | | | |
| carcinoma | | | | | |
| (cup) | | | | | |
| Uterus | F | 69 | 1663G>T | D555Y | Substitution |
| Adenoid cystic | M | 68 | 1664A>G | D555G | Substitution |
| carcinoma | | | | | |
| Adenoid cystic | M | 56 | 1664A>G | D555G | Substitution |
| carcinoma | | | | | |
| Lung non- | M | 81 | 1664A>C | D555A | Substitution |
| small cell lung | | | | | |
| carcinoma | | | | | |
| (nsclc) | | | | | |
| Prostate | M | 66 | 1664A>G | D555G | Substitution |
| Adenoid cystic | F | 55 | 1664A>G | D555G | Substitution |
| carcinoma | | | | | |
| Adenoid cystic | M | 53 | 1664A>C | D555A | Substitution |
| carcinoma | | | | | |
| Adenoid cystic | M | 67 | 1664A>G | D555G | Substitution |
| carcinoma | | | | | |
| Adenoid cystic | M | 68 | 1664A>G | D555G | Substitution |
| carcinoma | | | | | |
| Adenoid cystic | M | 50 | 1664A>C | D555A | Substitution |
| carcinoma | | | | | |
| Lung non- | M | 57 | 1665C>A | D555E | Substitution |
| small cell lung | | | | | |
| carcinoma | | | | | |
| (nsclc) | | | | | |
| Unknown | M | 53 | 1665_1666insG | D555_L556ins | Nonframeshift |
| primary | | | TGGAC | VD | Insertion |
| carcinoma | | | | | |
| (cup) | | | | | |
| Ovary | F | 28 | 1982_1983INS | K665_D666ins | Nonframeshift |
| | | | TTACTGCTCC | GYCSK | Insertion |
| | | | AAAGG | | |
| Salivary gland | F | 60 | 1989_1990insC | C663_S664ins | Nonframeshift |
| | | | GGCACAATT | RHNYC | Insertion |
| | | | ACTGC | | |
| Bladder | M | 61 | 1990_1991insA | C663_S664ins | Nonframeshift |
| | | | CGGCTACCT | YGYLYRHNYC | Insertion |
| | | | TTACCGGCA | | |
| | | | CAATTACTG | | |
| | | | CT | | |
| Breast | F | 77 | 1990_1991INS | C663_S664ins | Nonframeshift |
| | | | ACCGGCACA | YRHNYC | Insertion |
| | | | ATTACTGCT | | |
| Head and neck | M | 28 | 1990_1991insA | C663_S664ins | Nonframeshift |
| | | | CCTTTACCG | YLYRHNYC | Insertion |
| | | | GCACAATTA | | |
| | | | CTGCT | | |
| Adenoid cystic | F | 70 | 1991_1992insT | S664_K665ins | Nonframeshift |
| carcinoma | | | TACTGCTC | YCS | Insertion |
| Bladder | M | | 1992_1993insC | S664_K665ins | Nonframeshift |
| | | | GGCACAATT | RHNYCS | Insertion |
| | | | ACTGCTCC | | |
| Ovary | F | 38 | 1992_1993insC | S664_K665ins | Nonframeshift |
| | | | TTTACCGGC | LYRHNYCS | Insertion |
| | | | ACAATTACT | | |
| | | | GCTCC | | |
| Adenoid cystic | M | 44 | 1995_1996insC | K665_D666ins | Nonframeshift |
| carcinoma | | | AGGACGGCT | QDGYLYRHNYCSK | Insertion |
| | | | ACCTTTACC | | |
| | | | GGCACAATT | | |
| | | | ACTGCTCCA | | |
| | | | AA | | |
| Breast | F | 67 | 1996_1996+1I | D666_K667ins | Nonframeshift |
| | | | NSACAATTA | DNYCSK | Insertion |
| | | | CTGCTCCAA | | |
| | | | AG | | |
| Adenoid cystic | F | 66 | 3102_3103insA | T1034_V1035insN | Nonframeshift |
| carcinoma | | | AT | | Insertion |
| Adenoid cystic | M | 50 | 3116_3117insC | A1039_S1040insA | Nonframeshift |
| carcinoma | | | GC | | Insertion |
| Anus | F | 58 | 3124_3125insT | M1041_R1042insL | Nonframeshift |
| | | | GC | | Insertion |
| Adenoid cystic | F | 68 | 3124_3125insT | M1041_R1042insL | Nonframeshift |
| carcinoma | | | GC | | Insertion |
| Adenoid cystic | M | 51 | 3124_3125INS | M1041_R1042insL | Nonframeshift |
| carcinoma | | | TGC | | Insertion |
| Adenoid cystic | F | 26 | 3124_3125insT | M1041_R1042insL | Nonframeshift |
| carcinoma | | | GC | | Insertion |
| Ovary | F | 57 | 3124_3125insT | M1041_R1042insL | Nonframeshift |
| | | | GC | | Insertion |
| Ovary | F | 77 | 3130_3131INS | E1043_R1044insK | Nonframeshift |
| | | | AAA | | Insertion |
| Endometrial | F | 68 | 3130_3131insA | E1043_R1044insK | Nonframeshift |
| | | | AA | | Insertion |
| Urinary-neuro | F | 34 | 3131_3132insA | R1044_I1045insM | Nonframeshift |
| | | | AT | | Insertion |
| Breast | F | 59 | 3131_3132insA | R1044_I1045insM | Nonframeshift |
| | | | AT | | Insertion |
| Adenoid cystic | F | 59 | 3131_3132insA | R1044_I1045insM | Nonframeshift |
| carcinoma | | | AT | | Insertion |
| Ovary | F | 64 | 3131_3132insA | R1044_I1045insM | Nonframeshift |
| | | | AT | | Insertion |
| Endometrial | F | 73 | 3133_3134insG | R1044_I1045insR | Nonframeshift |
| | | | GA | | Insertion |
| Endometrial | F | 62 | 3137_3138insT | I1045_E1046insD | Nonframeshift |
| | | | GA | | Insertion |
| Adenoid cystic | M | 43 | 3138_3139insG | E1046_F1047ins | Nonframeshift |
| carcinoma | | | CCGCAAGCA | AASMRERIE | Insertion |
| | | | TGCGTGAGA | | |
| | | | GGATTGAG | | |
| Prostate | M | 67 | 3138_3139insC | E1046_F1047ins | Nonframeshift |
| | | | GTGAAAGGA | RERIE | Insertion |
| | | | TTGAG | | |
| Endometrial | F | 33 | 3138_3139insC | E1046_F1047ins | Nonframeshift |
| | | | GTGAGAGGA | RERIE | Insertion |
| | | | TTGAG | | |
| Adenoid cystic | F | 55 | 3143_3144insT | L1048_N1049insL | Nonframeshift |
| carcinoma | | | CT | | Insertion |
| Breast | F | 82 | 3141T>GTTG | F1047>LL | Nonframeshift |
| | | | | | Insertion |
| Breast | F | 57 | 3143_3144insT | L1048_N1049insL | Nonframeshift |
| | | | CT | | Insertion |
| Breast | F | 65 | 3143_3144insT | L1048_N1049insL | Nonframeshift |
| | | | CT | | Insertion |
| Breast | F | 42 | 3143_3144insT | L1048_N1049insL | Nonframeshift |
| | | | CT | | Insertion |
| Breast | F | 58 | 3143_3144insT | L1048_N1049insL | Nonframeshift |
| | | | CT | | Insertion |
| Breast | F | 51 | 3143_3144insT | L1048_N1049insL | Nonframeshift |
| | | | CT | | Insertion |
| Breast | F | 76 | 3143_3144insT | L1048_N1049insL | Nonframeshift |
| | | | CT | | Insertion |
| Adenoid cystic | M | 62 | 3143_3144insT | L1048_N1049insL | Nonframeshift |
| carcinoma | | | CT | | Insertion |
| Ovary | F | 68 | 3143_3144insT | L1048_N1049insL | Nonframeshift |
| | | | CT | | Insertion |
| Ovary | F | 70 | 3143_3144insT | L1048_N1049insL | Nonframeshift |
| | | | CT | | Insertion |
| Ovary | F | 79 | 3143_3144insT | L1048_N1049insL | Nonframeshift |
| | | | CT | | Insertion |
| Prostate | M | 73 | 3143_3144insT | L1048_N1049insL | Nonframeshift |
| | | | CT | | Insertion |
| Adenoid cystic | M | 63 | 3143_3144insT | L1048_N1049insL | Nonframeshift |
| carcinoma | | | CT | | Insertion |
| Adenoid cystic | F | 51 | 3143_3144insT | L1048_N1049insL | Nonframeshift |
| carcinoma | | | CT | | Insertion |
| Uterus | F | 46 | 3143_3144insT | L1048_N1049INSL | Nonframeshift |
| | | | CT | | Insertion |
| Uterus | F | 40 | 3143_3144insT | L1048_N1049INSL | Nonframeshift |
| | | | CT | | Insertion |
| Endometrial | F | 41 | 3143_3144insT | L1048_N1049INSL | Nonframeshift |
| | | | CT | | Insertion |
| Endometrial | F | 73 | 3143_3144insT | L1048_N1049INSL | Nonframeshift |
| | | | CT | | Insertion |
| Endometrial | F | 40 | 3143_3144insT | L1048_N1049INSL | Nonframeshift |
| | | | CT | | Insertion |
| Adenoid cystic | F | 61 | 3143_3144insT | L1048_N1049INSL | Nonframeshift |
| carcinoma | | | CT | | Insertion |
| Ovary | F | 78 | 3143_3144insT | L1048_N1049INSL | Nonframeshift |
| | | | CT | | Insertion |
| Salivary gland | F | 61 | 3143_3144insT | L1048_N1049INSL | Nonframeshift |
| | | | CT | | Insertion |
| Breast | F | 56 | 3145A>CTCG | N1049>LD | Nonframeshift |
| | | | | | Insertion |

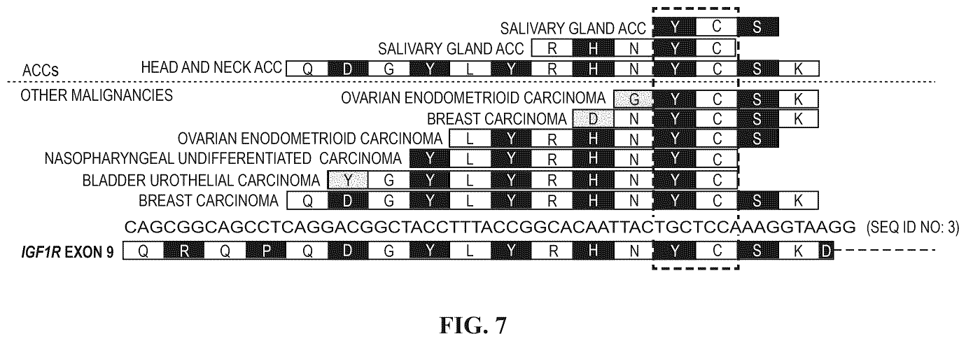

The fibronectin domain in exon 9 of IGF1R is a key structural component of the IGF1R peptide, in which a total of nine samples had non-frameshift insertions at this hotspot, including three ACCs. Insertions occurred in a three amino acid window in IGF1R exon 9 and ranged from three to 13 AAs in length (Tables 1-3). All were duplications or near duplications that differed from reference only by substitution of a single amino acid at the duplication breakpoint (FIG. 7). All cases shared duplication of Y262 and C263 with one to nine additional amino acids duplicated on either side.

Non-frameshift insertions were also clustered at the N-terminal end of the kinase domain, with insertion start sites spanning amino acids 1034-1049 (Tables 1-3). Variants at this hotspot were more frequent, with 12 ACCs and 25 non-ACC samples exhibiting non-frameshift insertions in this region. Kinase non-frameshifts were a mix of duplications and non-duplication insertions, and in all cases resulted in net addition of one amino acid. The most common individual variant was L1048dup (5 ACCs and 13 non-ACCs), followed by M141_R1042insL (3 ACCs and 2 non-ACCs), and R1044_I1045insM (1 ACC and 3 non-ACCs).

Of the two identified regions in which the identified IGF1R non-frameshift insertions most predominantly occur, the region of amino acid position 653-667 corresponds to a linker region connecting two α-CT motifs, wherein the lengthening of this linker was shown to reduce negative cooperativity and thus increase binding of IGF1 to IGF1R by ˜50%. The more frequent insertion hotspot at amino acid position 1034-1049, occurs within the tyrosine kinase domain. Thus, both regions have demonstrated impacts on the IGF1R signaling axis and activity. The results described herein identify for the first time that IGF1R alterations drive a subset of ACC that are negative for previously identified mutations, including the canonical MYB-NFIB fusion. Additionally, IGF1R provides a therapeutic target for the treatment of ACCs that display one or more of the identified hotspot IGF1R non-frameshift insertions described herein.

### Example 2—IGF1R Hotspot Non-Frameshift Insertions in Salivary Gland Neoplasms are Novel Biomarkers of ACC Diagnosis

In conjunction with the DNA sequencing and analysis described in Example 1 above, pathology reports were reviewed to extract patient data on age, sex, and oncologic diagnosis. The pathologic diagnosis of each case was confirmed on routine hematoxylin and eosin (H&E)-stained slides according to the diagnostic criteria specified in the 2017 World Health Organization Classification of Head and Neck Tumors.

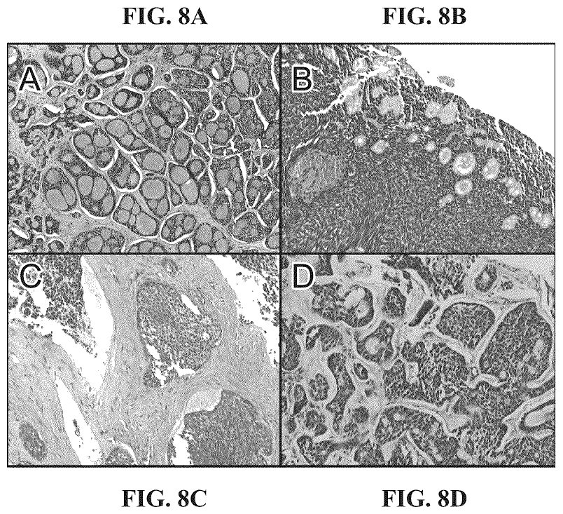

Upon identification of an association between ACC and hotspot alterations, the histology was retrospectively re-reviewed for all tumors of salivary, head and neck, breast, and lung origin that displayed an IGF1R non-frameshift insertion. Tumors were reclassified as ACC if they demonstrated areas with classic ACC histologic features that included a basaloid appearance, presence of cribriform architecture, and characteristic stroma (FIGS. 8A-D). Of the sections analyzed, 67% (2/3) of salivary tumors possessing an identified IGF1R non-frameshift insertion that were originally assigned a disease ontology of “salivary gland carcinoma, NOS” were actually either misclassified or underspecified ACC. Additional potentially similarly miscategorized cases were originally classified as other salivary gland neoplasms, such as lacrimal duct carcinoma. Upon closer review, 100% (14/14) of salivary tumors that displayed an identified hotspot IGF1R non-frameshift insertion showed classic histologic features of ACC.

These results demonstrate the clinical application and value of the use of these identified hotspot IGF1R non-frameshift insertions as a biomarker for ACC. Indeed, in the context of a head and neck or salivary gland primary, identification of a mutation is strongly suggestive of a diagnosis of ACC and may be particularly valuable as a molecular support for the diagnosis in challenging cases.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of treating a cancer, or delaying the progression of cancer, in an individual, comprising:
detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
administering an effective amount of an IGF1R-targeted therapy to the individual.

2. The method of claim 1, wherein the IGF1R-targeted therapy is administered to the individual in response to a determination of the presence of the non-frameshift insertion mutation or the substitution mutation.

3. A method of identifying a cancer in an individual as an adenoid cystic carcinoma comprising detecting an IGF1R polypeptide having a mutation or a nucleic acid molecule encoding the IGF1R polypeptide having the mutation in a sample from the individual, or acquiring knowledge of the presence of the IGF1R polypeptide having the mutation or the nucleic acid molecule encoding the IGF1R polypeptide having the mutation in the sample from the individual, wherein the mutation is a non-frameshift insertion mutation at or following any one or more of amino acids 653-667 of IGF1R or within a tyrosine kinase domain of IGF1R, or a substitution or insertion mutation at D555 of IGF1R,
wherein the presence of the mutation in IGF1R indicates an increased likelihood that the cancer is an adenoid cystic carcinoma.

4. The method of claim 3, wherein the cancer was previously identified as a cancer other than an adenoid cystic carcinoma.

5. The method of claim 3, further comprising identifying a basaloid appearance, presence of cribriform architecture, or stroma characteristic of an adenoid cystic carcinoma, within a histological image of the cancer.

6. The method of claim 3, further comprising administering an IGF1R-targeted therapy to the individual, thereby treating the cancer.

7. A method of treating an individual having cancer comprising:
selecting the individual for treatment based on the cancer having an IGF1R polypeptide having a mutation or a nucleic acid molecule encoding the IGF1R polypeptide having the mutation, wherein the mutation is a non-frameshift insertion mutation at or following any one or more of amino acids 653-667 of IGF1R or within a tyrosine kinase domain of IGF1R, or a substitution or insertion mutation at D555 of IGF1R; and
administering an effective amount of an IGF1R-targeted therapy to the selected individual.

8. The method of claim 1, wherein the cancer is a breast cancer, a head and neck cancer, an adenoid cystic carcinoma, an anal cancer, a bladder cancer, a uterine cancer, a lung cancer, a skin cancer, a neuroendocrine cancer, an ovarian cancer, or a prostate cancer.

9. The method of claim 1, wherein the cancer is an adenoid cystic carcinoma.

10. The method of claim 1, wherein the mutation is a non-frameshift insertion mutation at or following any one or more of amino acids 653-667 of IGF1R.

11. The method of claim 1, wherein the mutation is a non-frameshift insertion mutation at or following any one or more of amino acids 663-666 of IGF1R.

12. The method of claim 1, wherein the non-frameshift insertion mutation comprises a duplication of Y662 and C663.

13. The method of claim 1, wherein the non-frameshift insertion mutation comprises a duplication or a delins mutation.

14. The method of claim 1, wherein the mutation comprises a D555A mutation, a D555Y mutation, a D555E mutation, a D555N mutation, a D555G mutation, a D555_L556insVD mutation, a Q653_K665dup mutation, a Q653_S664dup mutation, a R659_C663dup mutation, a Y656_C663dup mutation, a L657_S664dup mutation, a N661_D666dup mutation, a Y662_S664dup mutation, a C663_S664insYRHNYC mutation, a C663_S664insRHNYC mutation, a C663_S664insYGYLYRHNYC mutation, a C663_S664insYRHNYC mutation, a C663_S664insYLYRHNYC mutation, S664_K665insYCS mutation, a S664_K665insRHNYCS mutation, a S664_K665insLYRHNYCS mutation, a K665_D666insGYCSK mutation, K665_D666insQDGYLYRHNYCSK mutation, a D666_K667insDNYCSK mutation, a T1034_V1035insN mutation, a A1039dup mutation, a A1039_S1040insA mutation, a M1041_R1042insL mutation, a R1042_E1046dup, a E1043_R1044insK mutation, a R1044_I1045insM mutation, a R1044_I1045insR mutation, a I1045_E1046insD mutation, a E1046_F1047insRERIE mutation, a E1046_F1047insAAMRERIE mutation, a F1047delinsLL mutation, a L1048dup mutation, a L1048_N1049insL mutation, or a N1049delinsLD mutation.

15. The method of claim 1, wherein the mutation is the non-frameshift insertion mutation within the tyrosine kinase domain of IGF1R.

16. The method of claim 1, wherein the non-frameshift insertion mutation within the tyrosine kinase domain of IGF1R is at or follows any one of amino acids 1034-1049 of IGF1R.

17. The method of claim 1, wherein the mutation is the substitution or insertion mutation at D555 of IGF1R.

18. The method of claim 1, wherein the cancer is negative for a MYB, MYBL1, or NOTCH1 driver mutation, or a MYB-NFIB fusion.

19. The method of claim 1, wherein the IGF1R-targeted therapy specifically targets IGF1R or an IGF1R ligand.

20. The method of claim 1, wherein the IGF1R-targeted therapy comprises kinase inhibitor that inhibits kinase activity of the IGF1R polypeptide.
